SEQUENCE LISTING (19 sequences)

SEQ ID NO: 1 - DNA, 1089 bp, Homo sapiens
  source 1..1089: mol_type = genomic DNA
atggcgcaaaccccggcgttcgataagccgaaagtggagctgcatgttcatctggacggtagcatcaaaccggaaaccattctgtattatggtcgtcgtcgtggtattgcgctgccggcgaacaccgcggaaggtctgctgaacgtgattggcatggacaagccgctgaccctgccggacttcctggcgaaatttgattactatatgccggcgattgcgggttgccgtgaggcgatcaagcgtattgcgtatgagttcgtggagatgaaggcgaaagaaggtgtggtttacgttgaggtgcgctatagcccgcacctgctggcgaacagcaaagttgaaccgatcccgtggaaccaagcggaaggcgacctgaccccggatgaggtggttgcgctggttggtcaaggcctgcaggaaggtgaacgtgattttggcgttaaggcgcgtagcattctgtgctgcatgcgtcaccagccgaactggagcccgaaagtggttgaactgtgcaagaaataccagcaacagaccgtggttgcgatcgacctggcgggtgatgaaaccatcccgggcagcagcctgctgccgggtcatgtgcaagcgtatcaggaagcggttaagagcggtatccaccgtaccgtgcatgcgggtgaggttggcagcgcggaagtggttaaagaggcggtggacattctgaaaaccgaacgtctgggtcacggctaccacaccctggaggatcaagcgctgtataaccgtctgcgtcaggaaaacatgcacttcgagatttgcccgtggagcagctatctgaccggtgcgtggaagccggacaccgaacacgcggttatccgtctgaaaaacgatcaagcgaactacagcctgaacaccgacgatccgctgattttcaagagcaccctggacaccgattatcagatgaccaaacgtgacatgggtttcaccgaggaagagtttaagcgtctgaacattaacgcggcgaaaagcagctttctgccggaagatgagaaacgtgagctgctggacctgctgtataaagcgtatggtatgccgccgagcgcgagcgcgggccaaaatctg

SEQ ID NO: 2 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 3 - DNA, 1056 bp, Mus musculus
  source 1..1056: mol_type = genomic DNA
atggcgcaaaccccggcgttcaataaaccgaaagtggagctgcacgttcatctggacggcgcgattaaaccggagaccattctgtatttcggtaagaaacgtggtattgcgctgccggcggacaccgtggaggaactgcgtaacatcattggcatggataagccgctgagcctgccgggtttcctggcgaaatttgactactatatgccggtgattgcgggctgccgtgaagcgatcaagcgtattgcgtacgaatttgttgagatgaaggcgaaagaaggtgtggtttacgttgaggtgcgctatagcccgcacctgctggcgaacagcaaagttgatccgatgccgtggaaccaaaccgagggtgatgtgaccccggatgatgtggttgatctggttaaccagggtctgcaagaaggcgagcaggcgttcggtatcaaagtgcgtagcattctgtgctgcatgcgtcaccaaccgagctggagcctggaagttctggagctgtgcaagaaatacaaccagaaaaccgtggttgcgatggacctggcgggtgatgaaaccatcgagggcagcagcctgtttccgggtcacgtggaagcgtatgagggtgcggttaagaacggcattcaccgtaccgtgcatgcgggtgaagttggcagcccggaagtggttcgtgaggcggtggacatcctgaaaaccgagcgtgttggtcacggctaccacaccattgaagatgaggcgctgtataaccgtctgctgaaggaaaacatgcacttcgaggtgtgcccgtggagcagctatctgaccggtgcgtgggacccgaaaaccacccacgcggtggttcgtttcaagaacgataaagcgaactacagcctgaacaccgacgatccgctgatctttaagagcaccctggacaccgattatcagatgaccaagaaagacatgggtttcaccgaggaagagtttaagcgtctgaacattaacgcggcgaaaagcagctttctgccggaagaggagaagaaggaactgctggaacgcctgtatcgtgagtatcag

SEQ ID NO: 4 - protein, 352 aa, Mus musculus
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 5 - protein, 369 aa, synthetic construct
  REGION 1..369: note = Plasmid Construct
MDWTWILFLVAAATRVHSAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGDVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 6 - protein, 352 aa, Mus musculus; identical to SEQ ID NO: 4

SEQ ID NO: 7 - protein, 352 aa, Mus musculus
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGDVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 8 - protein, 380 aa, synthetic construct
  REGION 1..380: note = IgE-hADA-1
MDWTWILFLVAAATRVHSAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 9 - DNA, 2999 bp, synthetic construct
  misc_feature 1..2999: note = Pvax vector sequence
  source 1..2999: mol_type = other DNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

SEQ ID NO: 10 - protein, 16 aa, synthetic construct
GSGSGGSGHHHHHHHH

SEQ ID NO: 11 - protein, 17 aa, synthetic construct
GSGLNDIFEAQKIEWHE

SEQ ID NO: 12 - DNA, 22 bp, synthetic construct
  source 1..22: mol_type = other DNA
cctctctctaatcagccctctg

SEQ ID NO: 13 - DNA, 21 bp, synthetic construct
  source 1..21: mol_type = other DNA
gaggacctgggagtagatgag

SEQ ID NO: 14 - DNA, 21 bp, synthetic construct
  source 1..21: mol_type = other DNA
cctgaaccttccaaagatggc

SEQ ID NO: 15 - DNA, 21 bp, synthetic construct
  source 1..21: mol_type = other DNA
ttcaccaggcaagtctcctca

SEQ ID NO: 16 - DNA, 21 bp, synthetic construct
  source 1..21: mol_type = other DNA
gcgtggtgttgctaaccttca

SEQ ID NO: 17 - DNA, 20 bp, synthetic construct
  source 1..20: mol_type = other DNA
aaggccacggtcatcagagt

SEQ ID NO: 18 - DNA, 23 bp, synthetic construct
  source 1..23: mol_type = other DNA
ctggaagatggacgtactttgtc

SEQ ID NO: 19 - DNA, 22 bp, synthetic construct
  source 1..22: mol_type = other DNA
cgacgaaggcgactaattttgc

(19) United States
(12) Patent Application Publication
(10) Pub. No.: US 2025/0312425 A1
(43) Pub. Date: Oct. 9, 2025

(54) COMPOSITIONS AND METHODS COMPRISING ADA-1 FOR WOUND HEALING AND VACCINE ENHANCEMENT

(71) Applicant: Drexel University, Philadelphia, PA (US)

(72) Inventors: Kara Lorraine Spiller, Philadelphia, PA (US); Elias El Haddad, Downingtown, PA (US); Michele Aileen Kutzler, Souderton, PA (US); Erin O'Brien, Philadelphia, PA (US); Gina M. Cusimano, Philadelphia, PA (US)

(21) Appl. No.: 19/172,548

(22) Filed: Apr. 7, 2025

Related U.S. Application Data
(60) Provisional application No. 63/575,388, filed on Apr. 5, 2024.

(51) Int. Cl. A61K 38/50; A61K 9/06; A61K 39/00; A61K 45/06; A61K 47/42; A61P 17/02; A61P 37/02

(52) U.S. Cl. CPC A61K 38/50; A61K 9/06; A61K 39/001154; A61K 45/06; A61K 47/42; A61P 17/02; A61P 37/02; C12Y 305/04004; A61K 2039/575

(57) ABSTRACT

Compositions and methods incorporating Adenosine deaminase-1 (ADA-1) as an adjuvant are provided. Polynucleotides encoding novel coding sequences, polypeptides, vectors containing same, and methods of use are provided. Compositions and methods incorporating Adenosine deaminase-1 (ADA-1) in a hydrogel are also provided. Compositions and methods comprising ADA-1 for treating wounds are also provided.

### CROSS-REFERENCE TO RELATED APPLICATIONS

This application claims the benefit under 35 USC 119(e) of U.S. Provisional Patent Application No. 63/575,388, filed Apr. 5, 2024, which is incorporated herein by reference.

### GRANT SUPPORT STATEMENT

This invention was made with government support under HL130037 awarded by the National Heart, Lung, and Blood Institute. The government has certain rights in the invention.

### INCORPORATION-BY-REFERENCE OF MATERIAL SUBMITTED IN ELECTRONIC FORM

The Contents of the electronic sequence listing (DRX-24-2531.xml; Size: 25,967 bytes; and Date of Creation: Apr. 7, 2025) is herein incorporated by reference in its entirety.

### BACKGROUND OF THE INVENTION

Several publications and patent documents are cited throughout the specification in order to describe the state of the art to which this invention pertains. Each of these citations is incorporated herein by reference as though set forth in full.

Despite the success of ART, HIV continues to spread to an estimated 1.5 million people per year. A prophylactic vaccine to prevent HIV acquisition remains a major unmet need in the field despite many years of failed attempts [9, 12].

Vaccine attempts using monomeric gp120 subunit vaccines have failed to induce bNAbs and did not prevent infection [13-15] necessitating the development of improved Env vaccine immunogens. Extensive research efforts have resulted in NLT antigens such as BG505-SOSIP.664 that are a stabilized, antigenic mimic of native, trimeric HIV Env designed to preferentially drive immune responses against broadly neutralizing epitopes while simultaneously occluding non-neutralizing epitopes [11][16, 17].

In addition to improved Env immunogens, there is a need for HIV vaccine adjuvants that reliably and robustly induce antibody somatic hypermutation to drive development of protective bNAbs [35]. This is exemplified in a study by Francica et al., where a gp140 Env protein vaccine was administered to NHPs in conjunction with eight different adjuvants. These adjuvants included Alum, MF59, TLR4 and TLR7 ligands, pIC:LC or immune stimulator complexes (ISCOMs) all of which are either clinically approved or in late-stage development. All adjuvants were found to enhance the magnitude of antibody responses however, none were able to increase somatic hypermutation (SHM) [36].

Adjuvants enhance vaccine immunogenicity, allow for antigen dose sparing, the generation of rapid and durable immune responses and the increase of overall vaccine effectiveness [20, 21]. Due to their immunostimulatory nature, adjuvants can be reactogenic and induce unwanted responses making it a challenge to strike a balance between effective adjuvants that are also suitable for human use. To this end, there are limited adjuvants that are clinically approved and the mechanism by which these adjuvants function is poorly understood. Only within the last two decades have novel adjuvants such as oil-in-water emulsions (MF59, AS03) gained approval [37]. Despite this, adverse side effects of oil-in-water emulsion adjuvants have raised concerns over their continued use. Although several other adjuvants have been tested preclinically and demonstrate high potency, safety and tolerability concerns have prevented these from achieving human licensure [20, 21, 22].

Dysfunctional wound healing in elderly patients makes advanced age one of the primary risk factors for developing a chronic, non-healing wound, particularly in the setting of type 2 diabetes and/or peripheral vascular disease. In young, healthy patients, healing progresses through tightly regulated phases orchestrated by cells of the immune system, particularly macrophages. It is well established that macrophages must exert a pro-inflammatory phenotype at early stages of wound healing prior to switching to a pro-reparative phenotype for proper wound resolution [5-8]. In elderly patients, chronic, low-grade inflammation typical of human aging increases the likelihood of the failed macrophage transition from inflammatory to reparative [9-11]. As a result, many therapeutic strategies have focused on inhibiting inflammation at later stages of wound healing [12-15]. However, it is now known that macrophages from aged mice or humans are hyporesponsive to pro-inflammatory stimuli, secreting lower levels of pro-inflammatory cytokines and signaling factors, which is one of the reasons why vaccines are less effective in the elderly [16-19].

What is needed are more effective adjuvant compositions for HIV-1, COVID-19, and other viruses and cancers. What is also needed are new therapeutic strategies for treating wound healing by inhibiting inflammation and accelerating wound healing.

### SUMMARY OF THE INVENTION

In one aspect of the invention, a hydrogel composition comprising an Adenosine deaminase-1 (ADA-1) peptide or a polynucleotide encoding an ADA-1 is provided. In another aspect of the invention, a method of treating a wound comprising administering a hydrogel composition comprising a polynucleotide encoding Adenosine deaminase-1 (ADA-1) is provided.

In certain embodiments, the ADA-1 peptide is SEQ ID NO: 2, a sequence sharing at least 90% identity therewith, SEQ ID NO: 4, or a sequence sharing at least 90% identity therewith. In certain embodiments, the polynucleotide encoding ADA-1 comprises the sequence of SEQ ID NO: 1, a sequence sharing at least 90% identity therewith, SEQ ID NO: 3, or a sequence sharing at least 90% identity therewith. In certain embodiments, the hydrogel is a gelatin-based hydrogel or a GelMA hydrogel. In certain embodiments, the hydrogel is formulated such that the ADA-1 is released over one or more days. In certain embodiments, the hydrogel is formulated such that the ADA-1 is released over 1-2 days. In certain embodiments, the hydrogel composition further comprises at least one additional therapeutic, such as an antibiotic, pain reliever, anti-inflammatory agent, or a steroid.

In certain embodiments, administering said hydrogel composition accelerates wound healing when compared to an untreated wound. In certain embodiments, the wound is a chronic wound, an acute wound, an open wound, a closed wound, a clean wound, a contaminated wound, an infected wound, a diabetic wound, an ulcer, a diabetic ulcer, a foot sore, or a skin sore.

In other aspects of the invention, methods of increasing vaccine response in a subject, comprising administering an ADA-1 polypeptide in combination with a vaccine composition to the subject, wherein administration enhances macrophage cytokine and chemokine profile, increases secretion of anti-inflammatory cytokines and/or chemokines in macrophages, increases the magnitude, durability, isotype switching and/or functionality of antigen-specific antibodies, increases antibody dependent complement deposition (ADCD), and/or induces bNAbs against the disease, are provided.

Still other aspects and advantages of these compositions and methods for making the compositions and using the compositions are described further in the following detailed description of the preferred embodiments thereof.

### BRIEF DESCRIPTION OF THE FIGURES

FIG. 1A-1D: ADA enhances HIV specific IgG and Antibody Dependent Complement Deposition (ADCD). Female BALB/c mice were immunized thrice with 1 ug or 5 ug of pBG505-TTT alone or with 10 μg of DNA-encoded ADA (pADA). Control mice were immunized with 15 ug of the empty backbone pVax (− control). Mice were bled at D14P1 and D21P2 immunizations prior to sacrifice at D14P3 immunizations (FIG. 1A). Sera was used to determine SOSIP specific IgG responses via ELISA. Area under the curve (AUC) was calculated for SOSIP specific IgG across all timepoints (D14P1, D21P2 and D14P3) (FIG. 1C). Sera was used to determine antibody dependent complement deposition (ADCD) (FIG. 1D). Data are representative of one experiment. Symbols represent individual animals, bars represent the mean, error bars represent the standard deviations. *p<0.05, **p<0.01, ***p<0.001, and ****p<0.0001 by Mann-Whitney-U test (FIG. 1B-1C) or Kruskal-Wallis ANOVA (FIG. 1D).

FIG. 2A-2B: VRC01 human monoclonal antibody ELISA assay control curves. Optical density curves of VRC01 human monoclonal antibody used to demonstrate consistency for ELISA assays. VRC01 curves were run on the same assay plates as data displayed in FIG. 1B (FIG. 2A-2B).

FIG. 3A-3B: ADA-1 did not enhance antibody neutralization at low doses of pBG505-TTT. Serum neutralization ID50s and Pearson correlation of SOSIP specific IgG and neutralization for animal experiments (FIG. 3A-3B). Symbols represent individual animals, bars represent the mean, error bars represent the standard deviations. *p<0.05, **p<0.01, ***p<0.001, and ****p<0.0001 by Mann-Whitney-U test.

FIG. 4A-4D: ADA enhances HIV specific antibody isotype switching in a dose sparing manner. Mice were immunized and bled as outlined in FIG. 1A. Sera was used to determine SOSIP specific IgG1 (FIG. 4A), IgG2a (FIG. 4B), IgG2b (FIG. 4C) and IgG3 (FIG. 4D) responses via ELISA. Pearson correlations were made between SOSIP specific IgG2a or IgG2b and IgG1 (E-F). Data are representative of one experiment. Symbols represent individual animals, bars represent the mean, error bars represent the standard deviations. *p<0.05, **p<0.01, ***p<0.001, and ****p<0.0001 by Mann-Whitney-U test (FIG. 4A-4D).

FIG. 5A-5C: ADA enhances HIV specific T cell responses. Splenocytes from immunized mice were harvested at D14P3 immunizations and stimulated with peptides spanning the HIV BG505 envelope protein. SOSIP specific cellular responses were evaluated via ELISpot assay (FIG. 5A). 3D structural (FIG. 5B) and linear (FIG. 5C) representation of BG505 peptide pools in relation to BG505 protein. Symbols represent individual animals, bars represent the mean, error bars represent the standard deviations. *p<0.05, **p<0.01, ***p<0.001, and ****p<0.0001 by two-way ANOVA.

FIG. 6A-6B: ADA-1 enhances IFNγ and TNFα expression in CD4⁺ T cells. Single cytokine expression for animal experiments outlined in FIG. 3.1A. Gating scheme is displayed in FIG. 7. IFNγ, TNFα and IL2 expression in CD4⁺ T cells (FIG. 6A) and CD8⁺ T cells (FIG. 6B). Symbols represent individual animals, bars represent the mean, error bars represent the standard deviations. *p<0.05, **p<0.01, ***p<0.001, and ****p<0.0001 by Mann-Whitney-U test.

FIG. 7: Gating strategy for intracellular cytokine staining and surface CD107a.

FIG. 8A-8E: ADA enhances HIV specific T cell polyfunctionality and degranulation. Spleenocytes from immunized mice were harvested at D14P3 immunizations and stimulated with peptides spanning the HIV BG505 envelope protein. SOSIP specific cellular responses were evaluated via intracellular cytokine staining (FIG. 8A-8D). CD107a expression was evaluated via flow cytometry (FIG. 8E). Data are representative of one experiment. Symbols represent individual animals, bars represent the mean, error bars represent the standard deviations. *p<0.05, **p<0.01, ***p<0.001, and ****p<0.0001 by Mann-Whitney-U test.

FIG. 9A-9D: ADA-1 enhances T effector memory cells. Frequency of total T central memory (CD62L⁺, CD44⁺) and T effector memory (CD62L⁻, CD44⁺) for the animal experiment outlined in FIG. 3.1A. Frequencies were determined from spleenocytes that were not stimulated with BG505 peptides. CD4⁺ T central memory (FIG. 9A) and effector memory (FIG. 9B). CD8⁺ T central memory (FIG. 9C) and effector memory (FIG. 9D). Symbols represent individual animals, bars represent the mean, error bars represent the standard deviations. *p<0.05, **p<0.01, ***p<0.001, and ****p<0.0001 by Mann-Whitney-U test.

FIG. 10: Gating strategy for total T central and effector memory cells. T central memory (CD62L⁺, CD44⁺) and T effector memory (CD62L⁻, CD44⁺) cells were quantified for the animal experiments.

FIG. 11: Gating strategy for activation induced marker (AIM) assay.

FIG. 12A-12C: ADA enhances HIV specific T cell activation and memory B cell frequencies. Splenocytes from immunized mice were harvested at D14P3 immunizations and stimulated with peptides spanning the HIV BG505 envelope protein. SOSIP specific cellular responses were evaluated via an activation induced marker (AIM) assay (FIG. 12A-12B). The spleenocytes were incubated with biotinylated SOSIP protein to evaluate frequencies of SOSIP specific memory B cells (FIG. 12C). Symbols represent individual animals, bars represent the mean, error bars represent the standard deviations. *p<0.05, **p<0.01, ***p<0.001, and ****p<0.0001 by or Mann-Whitney-U test.

FIG. 13 Gating strategy for SOSIP specific memory B cells.

FIG. 14A-14F: Higher doses of pBG505-TTT and protein co-immunization induce qualitative differences in HIV specific antibodies. Mice were immunized with 10 ug of pBG505 alone or with 10 ug pADA. A separate group of mice were immunized with both DNA as described and 10 ug of trimeric Env recombinant protein (rBG505-SOSIP) with adjuvants Alum or AddaVax. Mice were bled at D21P2 immunizations prior to sacrifice at D14P3 immunizations (FIG. 14A). Sera was used to determine SOSIP specific IgG via ELISA (FIG. 14B) and neutralization (FIG. 14C). Pearson correlations were performed for SOSIP specific IgG and neutralization ID50 (FIG. 14D-14F). Symbols represent individual animals, bars represent the mean, error bars represent the standard deviations. *p<0.05, **p<0.01, ***p<0.001, and ****p<0.0001 by Mann-Whitney-U test (FIG. 14B-14C).

FIG. 15: ADA-1 modestly increases SOSIP specific IgA. SOSIP specific IgA measured via ELISA for animal experiments outlined in FIG. 13A. Symbols represent individual animals, bars represent the mean, error bars represent the standard deviations. *p<0.05, **p<0.01, ***p<0.001, and ****p<0.0001 by Kruskal-Wallis Anova.

FIG. 16: Experimental design of macrophage stimulation with ADA-1. Human monocytes were isolated from healthy young (<65 yrs) and aged (≥70 yrs) PBMCs. Monocytes were polarized to M0 macrophages by culturing in the presence of MCSF for 5 days (at *). M0 macrophages were maintained by continued culturing with MCSF alone (M0 control) or were stimulated with 1 μM, 3 μM, 7 μM of ADA protein for 48 hrs (at # and \hat{ }). M0 macrophages were polarized to M1 macrophages by culturing with IFNγ and LPS for 48 hrs alone (M1 control) or were stimulated with 1 μM, 3 μM, 7 μM of ADA protein (at +). M0 and M1 macrophages were harvested, RNA was isolated and used for PCR analysis for cytokine (IL-6, TNFα) and chemokine (CCL22) gene expression (at ˜).

FIG. 17A-17C: ADA-1 rescues age associated deficits in TNFα gene expression. TNFα gene expression was measured in young (FIG. 17A) and aged (FIG. 17B) macrophages. M0 macrophages are represented in blue bars and M1 macrophages are represented in red bars. TNFα gene expression was compared between young and aged macrophages. Young macrophages are represented with square symbols and aged macrophages are represented with the circle symbols (FIG. 17C). Gene expression is displayed as fold change over the housekeeping gene RPS27A. Symbols represent individual donors, bars represent the mean, error bars represent the standard deviations. *p<0.05, **p<0.01, ***p<0.001, and ****p<0.0001 by Kruskal-Wallis ANOVA (FIG. 17A-17B) or Mann-Whitney-U test (FIG. 17C).

FIG. 18A-18C: ADA-1 enhances IL-6 gene expression in young and aged macrophages. IL-6 gene expression was measured in young (FIG. 18A) and aged (FIG. 18B) macrophages. M0 macrophages are represented in blue bars and M1 macrophages are represented in red bars. IL-6 gene expression was compared between young and aged macrophages. Young macrophages are represented with square symbols and aged macrophages are represented with the circle symbols (FIG. 18C). Gene expression is displayed as fold change over the housekeeping gene RPS27A. Symbols represent individual donors, bars represent the mean, error bars represent the standard deviations. *p<0.05, **p<0.01, ***p<0.001, and ****p<0.0001 by Kruskal-Wallis ANOVA (FIG. 18A-18B) or Mann-Whitney-U test (FIG. 18C).

FIG. 19A-19C: CCL22 gene expression is increased in aged macrophages. CCL22 gene expression was measured in young (FIG. 19A) and aged (FIG. 19B) macrophages. M0 macrophages are represented in blue bars and M1 macrophages are represented in red bars. CCL22 gene expression was compared between young and aged macrophages. Young macrophages are represented with square symbols and aged macrophages are represented with the circle symbols (FIG. 19C). Gene expression is displayed as fold change over the housekeeping gene RPS27A. Symbols represent individual donors, bars represent the mean, error bars represent the standard deviations. *p<0.05, **p<0.01, ***p<0.001, and ****p<0.0001 by Kruskal-Wallis ANOVA (FIG. 19A-19B) or Mann-Whitney-U test (FIG. 19C).

FIG. 20A-20D: Sequential HIV vaccination strategy. BNabs can be achieved during natural HIV infection in humans however this requires a high level of affinity maturation which occurs over the course of several years. Reverse vaccinology can be used to design immunogens based on known bNAbs (FIG. 20A). Inferred precursors of the bNAbs (also known as inferred germline bNAbs) do not generally bind to native-like trimers (NLTs), requiring the design of immunogens to activate precursor bNAbs B cells (germline-targeting immunogens such as BG505-SOSIP.GT1.1). To shepherd the antibody response towards mature bNAbs, a series of immunogens with increasing native trimer-like features are used. Finally, immunization is carried out with NLTs such as BG505-SOSIP.664 (FIG. 20B). Immunogen testing uses animal models expressing bNAbs precursor B cells (knock-in mice) or human antibody repertoires before moving into the clinic (FIG. 20C-20D).

FIG. 21: Mechanism of ADA-1. ADA-1 encoded DNA is taken up by cells at the injection site resulting in ADA-1 protein expression (at *). ADA-1 drives increased TNFα and IL-6 expression in macrophages (at **). ADA-1 driven TNFα production from macrophages can induce recruited monocytes to differentiate into DCs (at ***). ADA-1 and TNFα drives DC activation and maturation (at +). ADA-1 matured DCs increase IL-6 and IL-10 production (at ++). DCs that have taken up ADA-1 DNA migrate to secondary lymphoid organs and express ADA-1 protein (at +++). ADA-1 binds receptors on DCs and T cells to enhance immune synapse formation (at #) and enhance T cell proliferation and differentiation (at ##). ADA-1 enhances the function of TFH resulting in improved B cell help and presumably prolongs the interaction of TFH and GC B cells (at =). Prolonged TFH:GC B cell interaction facilitates enhanced adaptive immune responses including increased frequencies of antigen specific memory B cells (at ˜˜), increase antigen specific antibody magnitude, isotype switching, affinity, functionality (at ˜) and presumably increased SHM. ADA-1 matured DCs producing increased IL-6 that have migrated to secondary lymphoid organs can drive increased CD8⁺ T cell effector functions (at ′).

FIG. 22: ADA-releasing GelMA hydrogel rescues inflammation to heal a wound in aged skin.

FIG. 23: Receptor (IL-4Rα) for reparative cytokine IL-4 upregulated in response to inflammatory stimuli.

FIG. 24A-24C: Expression of key inflammatory markers in young and aged macrophages after treatment with ADA (FIG. 24A). These results were confirmed in murine macrophages (FIG. 24B-24C).

### DETAILED DESCRIPTION OF THE INVENTION

Provided herein are compositions and methods which employ adenosine deaminase-1 constructs, polynucleotides, and polypeptides as an adjuvant for use with various vaccines. ADA-1 fusion protein expression constructs have been developed for use in subjects in need thereof, including humans. Also provided herein are compositions and methods which employ adenosine deaminase-1 constructions, polynucleotides, polypeptides for use in the treatment of wounds. In certain embodiments, the ADA-1 polynucleotides or polypeptides are present in a hydrogel.

Since ADA-1 is critical to TFH programing, induces TFH differentiation, prolongs TFH survival [27] and TFH are necessary for germinal centers and are critical drivers SHM [30] the inventors hypothesized that ADA-1 could help drive SHM needed for an effective HIV vaccine. Provided herein are methods for improving HIV vaccine immunogens and adjuvants by evaluating a DNA vaccine encoding to BG505-SOSIP.664 (pBG505-TTT) adjuvanted with DNA encoded ADA-1.

The inventors further hypothesized that the co-delivery of plasmid-encoded ADA-1 (pADA) as a molecular adjuvant would improve the quality and quantity of immune responses to viral immunogens, including humoral immune responses to HIV-1 env DNA immunogens in a GC TFH-dependent manner. In vitro, it was observed that treatment of human mDCs with ADA-1 induced maturation and resulted in a cytokine/chemokine secretion profile which likely supports TFH differentiation. In vivo, in the context of a consensus clade B HIV env DNA immunogen, a significant increase in the levels of HIV-binding IgG in the serum of mice co-immunized with pADA was observed. These responses correlated with increased frequencies of GC TFH in the draining lymph nodes (DLNs) of vaccinated animals. Increases in autoantibody in the serum of these vaccinated animals were undetectable, indicating that pADA-mediated enhancement of GC responses is antigen-specific. As heterologous DNA prime-protein boost and simultaneous DNA-protein vaccination regimens have shown the most promise for the development of anti-HIV immunity, the ability of pADA to enhance GC activities in the context of a DNA-protein co-immunization regimen was evaluated. Again, addition of molecular ADA-1 to the DNA arm of this regimen resulted in superior HIV-binding antibody production. Finally, addition of pADA to the DNA arm of this regimen resulted in the production of heterologous tier-1 nAbs in vaccinated mice. These data indicate that pADA co-immunization enhances antigen-specific IgG in the serum of vaccinated animals in a GC TFH-dependent manner and does so without any enhancement of autoimmune antibodies. Thus, ADA-1 is a promising adjuvant that targets vaccine-induced GC responses.

### ADA-1 Vectors and Constructs

Adenosine deaminase-1 (ADA1 or ADA-1) is an intracellular enzyme, as well as an ecto-enzyme (cell surface-bound), of the purine metabolism pathway. ADA-1 exerts its functions through both enzymatic and non-enzymatic mechanisms. The enzymatic function of ADA-1 is achieved by irreversible catabolism of adenosine or 2′-deoxyadenosine into inosine or 2′-deoxyinosine via deamination1. In humans, functional mutations of ADA-1 lead to early-onset severe combined immunodeficiency (SCID), which is characterized by the loss of functional T, B, and NK lymphocytes, impaired both cellular and humoral immunity, and an extreme susceptibility to repeated and persistent infections which are often caused by “opportunistic” organisms.

In one aspect, provided herein are engineered polynucleotides that encode murine or human adenosine deaminase-1. In the case of ADA-1, the native gene employs tandem rare codons that can reduce the efficiency of translation or even disengage the translational machinery. The inventors increased the codon usage bias in E. coli by upgrading the CAI to 0.96. GC content and unfavorable peaks have been optimized to prolong the half-life of the mRNA. The Stem-Loop structures, which impact ribosomal binding and stability of mRNA, were broken. In addition, the inventors screened and successfully modified negative cis-acting sites. Thus, provided herein, in one embodiment, is a polynucleotide encoding human ADA-1 comprising the sequence of SEQ ID NO: 1, or a sequence sharing at least 90% identity therewith. In another embodiment, a polynucleotide encoding murine ADA-1 comprising the sequence of SEQ ID NO: 3, or a sequence sharing at least 90% identity therewith is provided. In another embodiment, the polynucleotide shares at least 91%, 92%, 93%, 94%, 95%, 96%, 97%, 98%, or 99% identity with SEQ ID NO: 1. In yet another embodiment, the polynucleotide shares at least 91%, 92%, 93%, 94%, 95%, 96%, 97%, 98%, or 99% identity with SEQ ID NO: 3.

In another aspect, an ADA-1 fusion protein is provided. In one embodiment, the fusion protein comprises an IgE signal sequence in combination with an ADA-1 polypeptide. In one embodiment, the fusion protein comprises the sequence of SEQ ID NO: 5, or a sequence sharing at least 90% identity therewith. In another embodiment, the fusion protein comprises the sequence of SEQ ID NO: 8, or a sequence sharing at least 90% identity therewith. In another embodiment, the fusion protein shares at least 91%, 92%, 93%, 94%, 95%, 96%, 97%, 98%, or 99% identity with SEQ ID NO: 5. In yet another embodiment, the fusion protein shares at least 91%, 92%, 93%, 94%, 95%, 96%, 97%, 98%, or 99% identity with SEQ ID NO: 8. In one embodiment, the fusion protein has the sequence of SEQ ID NO: 5 with a 234E→D substitution. In one embodiment, the fusion protein has the sequence of SEQ ID NO: 8 with a 234E→D substitution.

In another aspect, polynucleotides which encode the ADA-1 fusion proteins described herein, are provided. In one aspect, the polynucleotide comprises any sequence which encodes the polypeptide of SEQ ID NO: 5, or a sequence sharing at least 90%, 91%, 92%, 93%, 94%, 95%, 96%, 97%, 98%, or 99% identity therewith. In another embodiment, the polynucleotide comprises any sequence which encodes the polypeptide of SEQ ID NO: 8, or a sequence sharing at least 90%, 91%, 92%, 93%, 94%, 95%, 96%, 97%, 98%, or 99% identity therewith.

In another aspect, the nucleic acid sequences described herein may be engineered into any suitable vector. The term “vector” refers to a nucleic acid molecule into which a second nucleic acid molecule can be inserted for introduction into a host cell where it will be replicated, and in some cases expressed. In other words, a vector is capable of transporting a nucleic acid molecule to which it has been linked. Cloning vectors as well as expression vectors are contemplated by the term “vector”, as used herein. Certain vectors are capable of autonomous replication in a host into which they are introduced (e.g., vectors having a bacterial origin of replication can replicate in bacteria). Other vectors can be integrated into the genome of a host upon introduction into the host cell, and thereby are replicated along with the host genome. Vectors according to the invention can be made by methods well known to the person skilled in the art.

In one embodiment, the vector is a plasmid suitable for use as, or in combination with, a DNA vaccine. In one embodiment, the vector is the pVAX1™ plasmid vector from Invitrogen. pVAX1™ is a 3.0 kb plasmid vector designed for use in the development of DNA vaccines. The pVAX vector sequence is shown in SEQ ID NO: 9. The vector was constructed to be consistent with the Food and Drug Administration (FDA) document, “Points to Consider on Plasmid DNA Vaccines for Preventive Infectious Disease Indications”, published Dec. 22, 1996. Features of the vector allow high-copy number replication in E. coli and high-level transient expression of the protein of interest in most mammalian cells. The vector contains the following elements: human cytomegalovirus immediate-early (CMV) promoter for high-level expression in a wide range of mammalian cells; bovine growth hormone (BGH) polyadenylation signal for efficient transcription termination and polyadenylation of mRNA; and a kanamycin resistance gene for selection in E. coli. Other suitable plasmids are known in the art, and include pcDNA3.1. See, Gomez and Onate, Plasmid-Based DNA Vaccines, 10.5772/intechopen.76754, published Nov. 5, 2018, available from intechopen.com/books/plasmid/plasmid-based-dna-vaccines.

In another embodiment, the vector is a viral vector, such as an adenoviral (Ad) vector or adeno-associated viral (AAV) vector.

In addition to the coding sequences for ADA-1, the vector also includes conventional control elements necessary which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences may include, without limitation, a enhancer; transcription factor; transcription terminator; promoter; efficient RNA processing signals such as splicing and polyadenylation (polyA) signals; sequences that stabilize cytoplasmic mRNA, for example Woodchuck Hepatitis Virus (WHP) Posttranscriptional Regulatory Element (WPRE); sequences that enhance translation efficiency (i.e., Kozak consensus sequence); sequences that enhance protein stability; and when desired, sequences that enhance secretion of the encoded product.

The regulatory control elements typically contain a promoter sequence as part of the expression control sequences. Constitutive promoters, regulatable promoters [see, e.g., WO 2011/126808 and WO 2013/04943], tissue specific promoters, or a promoter responsive to physiologic cues may be used may be utilized in the vectors described herein. In one embodiment, the promoter is a CMV promoter.

Examples of constitutive promoters suitable for controlling expression of the therapeutic products include, but are not limited to chicken β-actin (CB) promoter, CB7 promoter, human cytomegalovirus (CMV) promoter, ubiquitin C promoter (UbC), the early and late promoters of simian virus 40 (SV40), U6 promoter, metallothionein promoters, EFlα promoter, ubiquitin promoter, hypoxanthine phosphoribosyl transferase (HPRT) promoter, dihydrofolate reductase (DHFR) promoter (Scharfmann et al., Proc. Natl. Acad. Sci. USA 88:4626-4630 (1991), adenosine deaminase promoter, phosphoglycerol kinase (PGK) promoter, pyruvate kinase promoter phosphoglycerol mutase promoter, the R-actin promoter (Lai et al., Proc. Natl. Acad. Sci. USA 86: 10006-10010 (1989)), the long terminal repeats (LTR) of Moloney Leukemia Virus and other retroviruses, the thymidine kinase promoter of Herpes Simplex Virus and other constitutive promoters known to those of skill in the art.

In one embodiment, the vector comprises one or more expression enhancers. In one embodiment, the vector contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another embodiment, the expression cassette further contains an intron, e.g., the chicken beta-actin intron. Other suitable introns include those known in the art, e.g., such as are described in WO 2011/126808. Examples of suitable polyA sequences include, e.g., rabbit binding globulin (rBG), SV40, SV50, bovine growth hormone (bGH), human growth hormone, and synthetic polyAs. Optionally, one or more sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16: 605-619).

### Vector Production

After recombinant plasmids are designed and constructed using known techniques, they are introduced into bacteria using electroporation (electric pulses) or chemical transformation (calcium chloride) methods. Transformed bacteria, usually E. coli, are cultured until reaching their logarithmic growth phase, allowing the production of multiple copies of the recombinant plasmid. Subsequently, the plasmids are extracted from these bacteria, avoiding contamination with lipopolysaccharide (LPS), a component of the E. coli outer membrane, which is pro-inflammatory and whose administration can produce adverse reactions in individuals vaccinated with this DNA. DNA concentrations obtained are adjusted in physiological saline or phosphate buffered saline (PBS) and stored for later administration. Methods of preparing plasmid-based vectors are known. See, e.g., pVAX1 manual, Invitrogen, available at assets.thermofisher.com/TFS-Assets/LSG/manuals/pvax1_man.pdf.

For use in producing a viral vector, the expression cassettes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art. Methods of preparing AAV-based vectors are known. See, e.g., US Published Patent Application No. 2007/0036760 (Feb. 15, 2007), which is incorporated by reference herein.

### Compositions and Uses

In another aspect, a composition is provided which includes one or more of the polynucleotides, fusion proteins, or vectors described herein. In one embodiment, the composition is a vaccine composition. In one embodiment, the composition is an adjuvant composition. An adjuvant is a component or composition that helps the body create a stronger immune response to a vaccine or other immunotherapy. In another embodiment, the composition is a hydrogel composition.

In one embodiment, the composition includes a vector comprising a polynucleotide encoding adenosine deaminase-1 (ADA-1) as an adjuvant. In one embodiment, the vector is a plasmid. In another embodiment, the vector is a viral vector.

In another embodiment, the composition comprises a polynucleotide that encodes an ADA-1 fusion protein comprising an IgE signal peptide and an ADA-1. In one embodiment, the ADA is a human ADA-1. In one embodiment, the ADA is a murine ADA-1. In one embodiment, the polynucleotide is SEQ ID NO: 1, or a sequence sharing at least 90% identity therewith. In yet another embodiment, the polynucleotide is SEQ ID NO: 3, or a sequence sharing at least 90% identity therewith. In another embodiment, the polynucleotide shares at least 91%, 92%, 93%, 94%, 95%, 96%, 97%, 98%, or 99% identity with SEQ ID NO: 1. In yet another embodiment, the polynucleotide shares at least 91%, 92%, 93%, 94%, 95%, 96%, 97%, 98%, or 99% identity with SEQ ID NO: 3. In one aspect, the polynucleotide comprises any sequence which encodes the polypeptide of SEQ ID NO: 5, or a sequence sharing at least 90%, 91%, 92%, 93%, 94%, 95%, 96%, 97%, 98%, or 99% identity with SEQ ID NO: 5. In another embodiment, the polynucleotide comprises any sequence which encodes the polypeptide of SEQ ID NO: 8, or a sequence sharing at least 90%, 91%, 92%, 93%, 94%, 95%, 96%, 97%, 98%, or 99% identity with SEQ ID NO: 8.

In one embodiment, the composition includes a pharmaceutically acceptable carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among nonionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

The vectors are administered in sufficient amounts provide a therapeutic benefit without undue adverse effects, or with medically acceptable physiological effects, which can be determined by those skilled in the medical arts. In certain embodiments, the vectors are formulated for intravenous delivery. In certain embodiments, the vectors are formulated for intramuscular delivery. In certain embodiments, the vectors are formulated for delivery via intranasal delivery devices for targeted delivery to nasal and/or nasopharynx epithelial cells. Other conventional and pharmaceutically acceptable routes of administration include, but are not limited to, direct delivery to a desired organ (e.g., lung), oral inhalation, intrathecal, intratracheal, intraarterial, intraocular, intravenous, intramuscular, subcutaneous, intradermal, and other parenteral routes of administration.

Dosages of the viral vector will depend primarily on factors such as the condition being treated, the age, weight and health of the patient, and may thus vary among patients. For example, a therapeutically effective human dosage of a viral vector is generally in the range of from about 25 to about 1000 microliters to about 25 mL of aqueous suspending liquid containing doses of from about 0.1 mg to about 100 mg plasmid DNA. A therapeutically effective human dosage of a viral vector is generally in the range of from about 25 to about 1000 microliters to about 25 mL of aqueous suspending liquid containing doses of from about 10⁹ to 4×10¹⁵ GC of vector.

The plasmid compositions can be formulated in dosage units to contain an amount of plasmid in the range of about 0.1 to about 100 mg plasmid DNA (to treat an average subject of 70 kg in body weight) including all integers or fractional amounts within the range, and preferably 0.1 to about 5 mg DNA for a human patient. In one embodiment, the dosage is about 0.1 mg plasmid DNA. In another embodiment, the dosage is about 0.5 mg plasmid DNA. In another embodiment, the dosage is about 0.75 mg plasmid DNA. In another embodiment, the dosage is about 1.0 mg plasmid DNA. In another embodiment, the dosage is about 1.5 mg plasmid DNA. In another embodiment, the dosage is about 2.0 mg plasmid DNA. In another embodiment, the dosage is about 2.5 mg plasmid DNA. In another embodiment, the dosage is about 3.0 mg plasmid DNA. In another embodiment, the dosage is about 3.5 mg plasmid DNA. In another embodiment, the dosage is about 4.0 mg plasmid DNA. In another embodiment, the dosage is about 4.5 mg plasmid DNA. In another embodiment, the dosage is about 5.0 mg plasmid DNA. In another embodiment, the dosage is about 5.5 mg plasmid DNA. In another embodiment, the dosage is about 6.0 mg plasmid DNA. In another embodiment, the dosage is about 6.5 mg plasmid DNA. In another embodiment, the dosage is about 7.0 mg plasmid DNA. In another embodiment, the dosage is about 7.5 mg plasmid DNA. In another embodiment, the dosage is about 8.0 mg plasmid DNA. In another embodiment, the dosage is about 8.5 mg plasmid DNA. In another embodiment, the dosage is about 9.0 mg plasmid DNA. In another embodiment, the dosage is about 9.5 mg plasmid DNA. In another embodiment, the dosage is about 10.0 mg plasmid DNA. In another embodiment, the dosage is about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 mg plasmid DNA.

The viral vector compositions can be formulated in dosage units to contain an amount of replication-defective virus that is in the range of about 10⁹ GC to about 10¹⁶ GC (to treat an average subject of 70 kg in body weight) including all integers or fractional amounts within the range, and preferably 10¹² GC to 10¹⁴ GC for a human patient.

These above doses may be administered in a variety of volumes of carrier, excipient or buffer formulation, ranging from about 25 to about 1000 microliters, or higher volumes, including all numbers within the range, depending on the size of the area to be treated, the plasmid concentration or viral titer used, the route of administration, and the desired effect of the method. In one embodiment, the volume of carrier, excipient or buffer is at least about 25 μL. In one embodiment, the volume is about 50 μL. In another embodiment, the volume is about 75 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 225 μL. In yet another embodiment, the volume is about 250 μL. In yet another embodiment, the volume is about 275 μL. In yet another embodiment, the volume is about 300 μL. In yet another embodiment, the volume is about 325 μL. In another embodiment, the volume is about 350 μL. In another embodiment, the volume is about 375 μL. In another embodiment, the volume is about 400 μL. In another embodiment, the volume is about 450 μL. In another embodiment, the volume is about 500 μL. In another embodiment, the volume is about 550 μL. In another embodiment, the volume is about 600 μL. In another embodiment, the volume is about 650 μL. In another embodiment, the volume is about 700 μL. In another embodiment, the volume is between about 700 and 1000 μL.

In one embodiment, the composition includes a hydrogel and adenosine deaminase-1 (ADA-1) peptide or a polynucleotide encoding ADA-1. In one embodiment, the hydrogel is a gelatin-based hydrogel. In one embodiment, the hydrogel is a GelMA hydrogel. In certain embodiments, the hydrogel restores macrophage phenotype transition.

Hydrogels have found application in many biomedical applications owing to their high water content, mechanical properties and good biocompatibility. A particularly useful application of hydrogels is where they are formulated to comprise a biologically active agent, for example, a drug, which can diffuse out from the hydrogel in a controlled manner when in implanted in a subject. Such hydrogels can be designed to degrade in situ after release of the biologically active agent to assist with clearance from the subject.

By the agent being “biologically active” means it is intended for use in the diagnosis, cure, mitigation, treatment, prevention or modification of a state in a biological system. For example, the agent may be a drug that is used to therapeutically to treat or prevent a disease state in humans or other animal species. In certain embodiments the biologically active agent is a ADA-1.

In certain embodiments, the hydrogel composition comprises an ADA-1 fusion protein or a polynucleotide that encodes an ADA-1 fusion protein. In one embodiment, the ADA is a human ADA-1. In one embodiment, the ADA is a murine ADA-1. In one embodiment, the polynucleotide is SEQ ID NO: 1, or a sequence sharing at least 90% identity therewith. In one embodiment, the ADA-1 peptide is SEQ ID NO: 2, or a sequence sharing at least 90% identity therewith. In yet another embodiment, the polynucleotide is SEQ ID NO: 3, or a sequence sharing at least 90% identity therewith. In one embodiment, the ADA-1 peptide is SEQ ID NO: 4, or a sequence sharing at least 90% identity therewith. In another embodiment, the polynucleotide shares at least 91%, 92%, 93%, 94%, 95%, 96%, 97%, 98%, or 99% identity with SEQ ID NO: 1. In another embodiment, the ADA-1 peptide shares at least 91%, 92%, 93%, 94%, 95%, 96%, 97%, 98%, or 99% identity with SEQ ID NO: 2. In yet another embodiment, the polynucleotide shares at least 91%, 92%, 93%, 94%, 95%, 96%, 97%, 98%, or 99% identity with SEQ ID NO: 3. In another embodiment, the ADA-1 peptide shares at least 91%, 92%, 93%, 94%, 95%, 96%, 97%, 98%, or 99% identity with SEQ ID NO: 4. In one aspect, the polynucleotide comprises any sequence which encodes the polypeptide of SEQ ID NO: 5, or a sequence sharing at least 90%, 91%, 92%, 93%, 94%, 95%, 96%, 97%, 98%, or 99% identity with SEQ ID NO: 5. In another embodiment, the polynucleotide comprises any sequence which encodes the polypeptide of SEQ ID NO: 8, or a sequence sharing at least 90%, 91%, 92%, 93%, 94%, 95%, 96%, 97%, 98%, or 99% identity with SEQ ID NO: 8.

In certain embodiments, the biologically active agent is released from the hydrogel in a sustained and controlled manner. The term “sustained release” refers to slow releasing of a specific substance at a programmed rate to deliver the drug for a prolonged period of time. In certain embodiments, the biologically active agent is released for 1-2 days in an in vitro measurement. In certain embodiments, the free drug is released within the first hour after administration with the remaining biologically active agent being release over at least 2 days. In certain embodiments, the release of the remaining biologically active agent occurs over 1-15 days or 1-2 days. In certain embodiments, the hydrogel releases the biologically active agent over at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, or at least 15 days.

In certain embodiments, the hydrogel, described herein, is administered with one or more additional therapeutics. In certain embodiments, the therapeutic is an antibiotic, a pain reliever, an anti-inflammatory agent, or a steroid.

The term “antibiotic” can refer to a substance that controls the growth of bacteria, fungi, or similar microorganisms, wherein the substance can be a natural substance produced by bacteria or fungi, or a chemically/biochemically synthesized substance (which may be an analog of a natural substance), or a chemically modified form of a natural substance. In general, any antibiotic can be used with the disclosed composition or methods. Examples of antibiotics that can be used include but are not limited to aminoglycosides (such as amikacin, gentamicin, kanamycin, neomycin, netilmicin, streptomycin, tobramycin, and paromomycin); ansamycins (such as geldanamycin, and herbimycin); carbacephems (such as loracarbef, ertapenem, doripenem, imipenem/cilastatin, and meropenem); cephalosporins (such as cefadroxil, cefazobn, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, and ceftobiprole); glycopeptides (such as teicoplanin and vancomycin); macrolides (such as azithromycin, clarithromycin, dirithromycin, erythromycin, roxithromycin, troleandomycin, telithromycin, and spectinomycin); monobactams (such as aztreonam); penicillins (such as amoxicillin, ampicillin, azlocillin, carbenicillin, cloxacillin, dicloxacillin, flucloxacillin, mezlocillin, meticillin, amoxycillin, clavamox, clavulanic acid, nafcillin, oxacillin, penicillin, piperacillin, and ticarcillin); peptides (such as bacitracin, colistin, and polymyxin b); quinolones (such as ciprofloxacin, enoxacin, gatifloxacin, levofloxacin, lomefloxacin, moxifloxacin, norfloxacin, ofloxacin, trovafloxacin, grepafloxacin, and sparfloxacin); sulfonamides (such as mafenide, prontosil (archaic), sulfacetamide, sulfamethizole, sulfanilimide (archaic), sulfasalazine, sulftsoxazole, trimethoprim, and trimethoprim-sulfamethoxazole); tetracyclines (such as demeclocycline, doxycycline, minocycline, oxy tetracycline, and tetracycline); and others (such as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin, rifampicin, thiamphenicol, and tinidazole) or combinations thereof.

The term “pain reliever” or “pain relieving agent” can refer to one having an action of relieving pain. Non-limiting examples of pain relievers, can include acetaminophen, ibuprofen, ketoprofen, diclofenac, naproxen, aspirin, and combinations thereof, as well as prescription analgesics, non-limiting examples of which include propyxhene HCl, codeine, mepridine, and combinations thereof.

The term “anti-inflammatory agent” refers to any composition or compound that reduces inflammation or swelling. Anti-inflammatory agents include, but are not limited to, acetaminophen, aspirin, celecoxib, diclofenac, diflunisal, flurbiprofen, ibuprofen, indomethacin, ketoprofen, ketorolac, meclofenamate, meloxicam, methyl salicylate, nabumetone, naproxen, oxaprozin, piroxicam, sulindac, tolmetin and trolamine.

The term “steroid” refers to a class of compounds used to treat inflammation and relieve swelling. Non-limiting examples of steroids include but are not limited to Dexamethasone, Methylprednisolone, Betamethasone Propionate, Betamethasone Sodium Phosphate, Diprospan (Betamethasone disodium phosphate and Betamethasone dipropionate), or another anti-inflammatory mineral corticosteroid or glucocorticosteroid.

Methods suitable for assessing the amount of ADA-1 in a sample using antibodies that bind to hADA-1 include those of Enzyme Linked Immunosorbent Assay (ELISA). Specifically, NOVUS Human Adenosine Deaminase/ADA ELISA Kit (Chemiluminescence) Kit, which can specifically detect human ADA in various samples such as serum, plasma and other biological fluids. The kit is utilized according to manufacturing instruction.

### Methods of Use

In another aspect, a method of increasing vaccine response in a subject is provided. The method includes providing a composition comprising ADA-1 in combination with a vaccine composition. In one embodiment, the method includes administering the polynucleotide, vector, fusion protein, or composition as described herein. In one embodiment, the composition is administered intramuscularly.

In certain embodiments, administration of the composition enhances macrophage cytokine and chemokine profile. In certain embodiments administration increases secretion of anti-inflammatory cytokines and/or chemokines. In certain embodiments, TNFα, IL-6, IFNγ, and/or IL2 expression in the subject when compared to a subject treated with just the vaccine composition. In certain embodiments, administration modulates CCL17, CCL22, CCL24 and/or CXCL13 expression and/or increases the magnitude, durability, isotype switching and/or functionality of antigen-specific antibodies. In certain embodiments, administration increases antibody dependent complement deposition (ADCD) and/or drives expression of IgG2a or IgG2b.

In certain embodiments, the vaccine composition is a COVID-19 vaccine or an HIV-1 vaccine. In certain embodiments, the vaccine composition is an HIV-1 vaccine and administration induces bNAbs against HIV.

In one embodiment, the subject is desirably a human. In another embodiment, the subject is an older adult, e.g., over the age of 40. In another embodiment, the subject is at least 45, 50, 55, or 60 years of age. In yet another embodiment, the subject is a senior adult, i.e., over 60 years of age. In certain embodiments, the subject is immunocompromised. In certain embodiments, the immunocompromised subject includes without limitation, the elderly, patients undergoing chemotherapy, patients having an autoimmune disease (e.g. diabetes), and patients having cancer.

The ADA-1 adjuvant compositions are useful with various vaccine compositions. In one embodiment, the vaccine composition is a DNA vaccine composition. In one embodiment, the vaccine composition is for COVID-19. In another embodiment, vaccine composition is for HIV-1.

The ADA-1 adjuvant composition may be administered prior to, concurrently with, or after a vaccine composition. In one embodiment, the ADA-1 composition and vaccine composition are administered approximately simultaneously.

Still further provided are uses of compounds described herein in the treatment of a wound. Still further provided are uses of the compounds in the preparation of medicaments useful in the treatment of a wound. In certain embodiments, the compounds are present in a hydrogel when treating a wound. Still further provided is the use of one of more of the compounds described herein to treat a wound. The treatment may comprise any of the methods described herein.

The methods described herein may be used for any type of wound. In specific embodiments, the wound may be a chronic wound, an acute wound, an open wound, a closed wound, a clean wound, a contaminated wound, an infected wound, a diabetic wound, an ulcer, a diabetic ulcer, a foot sore, or a skin sore. However, the embodiments described herein are not so limited.

The term “wound” is intended to refer to an injury to living tissue of a subject. Unless otherwise noted, embodiments referring to a wound include embodiments where the wound is one in which the skin is cut or broken, including surgical wounds. Further, embodiments referring to a wound include embodiments where the epidermis is broken. Still further, embodiments referring to a wound include embodiments where the dermis is cut or broken. In still other embodiments referring to a wound, the wound is a tissue other than of the skin, including internal surgical wounds.

The wound may be acute. The term “acute wound” refers to a wound which heals consistent with the timing or process conventional to the type and severity of the wound for the species of the subject.

The wound may be chronic, hard-to-heal, or refractory. The term “chronic wound” refers to a wound which does not heal consistent with the timing or process conventional to the type and severity of the wound for the species of the subject. The terms “hard-to-heal” or “refractory” refer to a wound which does not heal using conventional therapies available as of the filing date of this application.

The wound may also be associated with a metabolic disease or other disease in which natural healing is diminished. The wound may be a diabetic wound. The wound may be a diabetic ulcer. The wound may be a wound of a patient with Type I or Type II diabetes. The term “diabetic wound” refers to any wound in an individual having diabetes. The terms “disease”, “disorder”, or “condition” are used herein to refer to any manifestations, symptoms, or combination of manifestations or symptoms, recognized or diagnosed as connected with a chronic wound, hard-to-heal wound, or diabetic wound.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

Generally, when referring to “identity”, “homology”, or “similarity” between two different nucleic acid or amino acids, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

As used herein with respect to vaccine compositions, an “effective amount” refers to the amount of the ADA-1 composition which provides sufficient levels of ADA-1 to increase the immune response of a subject to a vaccine composition. An effective amount may be determined based on an animal model, rather than a human patient. Examples of a suitable murine model are described herein.

As used herein with respect to wound healing, an “effective amount” refers to the amount of the ADA-1 composition which provides sufficient levels of ADA-1 to increase wound healing and/or improve symptoms associated with said wound. An effective amount may be determined based on an animal model, rather than a human patient. Examples of a suitable murine model are described herein.

As described above, the term “about” when used to modify a numerical value means a variation of ±10%, unless otherwise specified.

As used throughout this specification and the claims, the terms “comprise” and “contain” and its variants including, “comprises”, “comprising”, “contains” and “containing”, among other variants, is inclusive of other components, elements, integers, steps and the like. The term “consists of” or “consisting of” are exclusive of other components, elements, integers, steps and the like.

It is to be noted that the term “a” or “an”, refers to one or more, for example, “an enhancer”, is understood to represent one or more enhancer(s). As such, the terms “a” (or “an”), “one or more,” and “at least one” is used interchangeably herein.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

### Example I: Adenosine Deaminase-1 Enhances HIV Specific Humoral and Cellular Responses to a Native-Like HIV Envelope Trimer DNA Vaccine

There is currently no prophylactic vaccine available for human immunodeficiency virus (HIV). Research efforts have resulted in improved immunogens that mimic the native envelope (Env) glycoprotein structure. Recently, a novel triple tandem trimer (TTT) platform has been used to generate a plasmid encoding Env immunogen (pBG505-TTT) that expresses only as trimers, making it more suitable for nucleic acid vaccines. We have previously demonstrated that adenosine deaminase-1 (ADA-1) is critical to the T follicular helper (TFH) function and improves vaccine immune responses in vivo. In this study, we demonstrate that co-delivery of plasmid-encoded adenosine deaminase 1 (pADA) with an HIV envelope trimer DNA vaccine, pBG505-TTT, enhances the magnitude, durability, isotype switching and functionality of HIV-specific antibodies in a dose-sparing manner. Co-delivery of the molecular immune modulator ADA-1 also enhances HIV-specific T cell polyfunctionality, activation, and degranulation as well as memory B cell responses. These data demonstrate that pADA enhances HIV-specific cellular and humoral immunity, making ADA-1 an effective immune modulator for HIV-targeting vaccines.

Human immunodeficiency virus (HIV) remains a prominent global health threat for which no prophylactic vaccine is available. Although there have been many efforts to develop an HIV vaccine, its development has proven to be challenging. Due to the high sequence diversity and multiple routes of transmission of HIV, it is critical for an effective HIV vaccine to induce both circulating and mucosal broadly neutralizing antibodies (bNAbs) capable of neutralizing most HIV circulating strains. Properties of the HIV envelope (Env) protein, used for viral entry, pose additional challenges. The HIV Env protein is extremely metastable and heavily glycosylated, resulting in very little access to immune recognition of neutralizing epitopes and subsequent immune evasion [10, 11].

Vaccine attempts using monomeric gp120 subunits have failed to induce bNAbs and did not prevent infection [13-15], necessitating the development of improved envelope vaccine immunogens. Extensive research efforts have resulted in immunogens, including SOSIP trimers, that closely mimic the native Env glycoprotein structure and consistently induce autologous neutralizing responses [11, 18, 19]. Previous studies using protein BG505-SOSIP vaccination have focused on humoral characterization with little cellular characterization, particularly CD8⁺ T cell responses[42, 43]. Recently, a novel triple tandem trimer (TTT) platform has been used to generate a plasmid encoding Env immunogen (pBG505-TTT) that expresses BG505 trimers, making it more suitable for nucleic acid vaccines [44]. A recent study with another native-like Env immunogen MD39, showed that MD39-encoding DNA vaccination elicited enhanced Env-specific humoral and cellular responses compared to MD39 protein. These results provided evidence that a DNA vaccine platform could be advantageous for driving anti-HIV responses over a protein subunit vaccine [45].

A novel molecular immune modulator, adenosine deaminase-1 (ADA-1) enhances the magnitude and durability of both cellular and humoral vaccine induced immune responses in vivo [25, 31, 32]. ADA-1 is necessary for normal immune function as exemplified by the severe-combined immunodeficiencies (SCID) that results from ADA-1 loss-of-function mutations [23]. Pegylated ADA is used therapeutically as an enzyme replacement therapy for the treatment of ADA-SCID [29]. The enzymatic function of ADA-1 is to catalyze the non-reversable deamination of adenosine into inosine therefore regulating both intracellular and extracellular adenosine concentrations. Independent of its enzymatic function, ADA-1 has also been shown to promote T cell proliferation and differentiation [28, 38, 39]. Importantly, we have demonstrated that ADA-1 prolongs TFH survival and induces TFH differentiation making it unique in boosting germinal center (GC) function resulting in increased quality of antigen specific adaptive immune responses to vaccines [27, 31].

TFH cells are necessary for germinal centers and are critical drivers of processes such as somatic hypermutation, affinity maturation and isotype switching in B cells. BNAbs against HIV exhibit high levels of somatic hypermutation and extensive affinity maturation [30]. Circulating TFH frequencies and function have been correlated with bNAbs [30, 46] and when administered passively, bNAbs against HIV have been shown to be protective against HIV acquisition in both humans and non-human primates (NHP) [47, 48]. In addition to antibody (Ab) neutralization, Fcγ receptor (FcγR)-mediated Ab function such as Ab dependent complement deposition (ADCD) has been shown to decrease HIV replication and may play a role in HIV protection [49-54]. ADCD has been found to be primarily driven by non-neutralizing antibodies (nNAbs) in the context of HIV and such nNAbs have been found to act together with CD8⁺ T cells to confer heterosubtypic immunity in the context of other viral infections [55, 56].

CD8⁺ T cell function during chronic viral infections has been found to be sustained by TFH cells producing IL-21 [57]. CD8⁺ T cells present in elite controllers of HIV infection have been found to establish immunological synapses with infected CD4⁺ T cells, leading to IFN-γ production, degranulation, and the elimination of the target cells [58]. Vaccine strategies designed to enhance CD8⁺ T cell frequency and effector function have also demonstrated protective HIV immunity in NHP [59]. These data demonstrate the importance of CD8⁺ T cell responses in an effective HIV vaccine and how vaccine strategies targeting TFH cells can drive both humoral responses and cellular responses that are critical for protective HIV immunity.

Since ADA-1 enhances TFH cell differentiation and function [27], and TFH cells are central to many aspects critical for an effective anti-HIV humoral and cellular response, we believe ADA-1 would serve as a promising immune modulator for HIV targeting vaccines. We therefore hypothesized that ADA-1 combined with the improved HIV envelope antigen pBG505-TTT would result in enhanced magnitude and quality of both humoral and cellular HIV specific responses.

To evaluate this hypothesis, mice were immunized with pBG505-TTT alone or in combination with plasmid encoded ADA-1 (pADA). To understand how ADA-1 functions as an immune modulator in relation to more classically used adjuvants such as Alum and MF59, another cohort of mice were co-immunized with pBG505-TTT with or without pADA, recombinant BG505 protein (rBG505) and either Alum or an MF59-like adjuvant AddaVax. ADA-1 was found to enhance HIV specific humoral responses as demonstrated by increased SOSIP-specific antibody magnitude, durability, isotype switching and functionality. pADA co-immunization also resulted in enhanced HIV specific cellular responses including increased T cell cytokine production, polyfunctionality, and activation as well as memory B cell responses. In comparison to other adjuvants, pADA was able to enhance Ab neutralization compared to alum while AddaVax induced robust Ab neutralization with no further enhancement with pADA. This study indicates that ADA-1 can be used as an effective immune modulator for HIV nucleic acid-based vaccine strategies.

### Materials and Methods

### DNA Plasmid Preparation

The untagged BG505 SOSIP.v8.1 gene, used to produce Env protein for vaccination, was derived from the previously described BG505 SOSIPv5.2 sequence [60] by introducing a subset of the MD39 trimer-stabilizing mutations (519S, 568D, 570H and 585H, HxB2 numbering) [61] and a stop codon after residue 664. His-tagged and avi-tagged BG505 SOSIP.v8.1 genes, used to produce proteins for immune responses characterization, included C-terminal sequences encoding GSGSGGSGHHHHHHHH (SEQ ID NO: 10) and GSGLNDIFEAQKIEWHE (SEQ ID NO: 11) peptides, respectively. The underlined GS residues were encoded by a BamHI restriction site for cloning purposes. The untagged BG505 triple tandem trimer (TTT) gene, encoded in the Env DNA immunogen, was designed as previously described [44], by concatenating three single-chain BG505 SOSIP.v8.4 protomers via 11-residue glycine-serine flexible linkers. This gene included a stop codon after position 664 of the third protomer. Constructs were generated by site-directed mutagenesis or by restriction-ligation of genes codon-optimized and synthesized by Genscript (Piscataway, USA) into a pPI4 plasmid. The BG505 TTT-encoding pPI4 plasmid used for vaccination was purified from heat-shock transformed DH5a competent cells (ThermoFisher Scientific, Cat: 18265017) using an NucleoBond Xtra Midi endotoxin-free (EF) kit (Macherey-Nagel), resuspended in EF water and stored at −20° C. until use.

A codon-optimized DNA plasmid encoding murine ADA-1 (pADA) was subcloned into the pVax expression vector and produced commercially (Genscript, Piscataway, NJ). In vitro studies revealed the expression of the ADA proteins after transfection of cell lines with the vaccine construct as previously described [25, 40].

### Protein Production

BG505 SOSIP.v8.1 proteins (untagged, his-tagged and avi-tagged) were expressed in HEK293F cells and purified by PG151 immunoaffinity chromatography, similar to previously described [62]. Briefly, HEK293F cells (Invitrogen, Cat: R79009) maintained in FreeStyle Expression Medium (Gibco) were transiently transfected with Env and furin expression plasmids in a 4:1 (w/w) Env to furin ratio. For transfection, the DNA mix was incubated with PEImax (Polysciences Europe GmBH, Eppelheim, Germany) in a 3:1 (w/w) PEImax to DNA ratio and then added to the supernatant of cells at a density of 0.8-1.2 million cells/mL. Five to seven days after transfection, supernatants were harvested, centrifuged and vacuum-filtered using 0.22 μm Steritops (Millipore, Amsterdam, The Netherlands).

Proteins in the filtered supernatants were captured on PGT151-coupled CNBr-activated sepharose 4B beads (GE Healthcare) by flowing at 0.5-1.0 mL/min or overnight rolling incubation at 4° C. Subsequently, the beads were immobilized in Econo-Column chromatography columns (Biorad) and washed with three column volumes of a 0.5 M NaCl 20 mM Tris HCl pH 8.0 solution. After elution with 3 M MgCl2 pH 7.5, proteins were buffer-exchanged to TN75 (75 mM NaCl, 20 mM Tris HCl pH 8.0) or PBS buffers using Vivaspin20 MWCO 100 kDa ultrafiltration units (Sartorius, Gottingen, Germany). After purification, avi-tagged proteins were biotinylated using a BirA500 biotin-ligase reaction kit (Avidity, Aurora, USA) according to manufacturer's instructions. Excess biotin was removed by ultrafiltration with Vivaspin6 MWCO 10 kDa filters (Sartorius, Gottingen, Germany). Protein concentrations were determined using a NanoDrop One spectrophotometer (ThermoFisher Scientific) and molecular weight and extinction coefficient values calculated with the ProtParam webtool (Expasy). Purified proteins were filter sterilized and stored at −80° C. until use. Untagged BG505 SOSIP.v8.1 protein was used for immunizations, his-tagged protein was used for ELISA assays, and avi-tagged biotinylated protein was used for memory B cell flow cytometry analysis.

### Immunizations

Female BALB/c mice aged 6-8 weeks were immunized in the tibialis anterior (TA) muscle with 20-40 μl of the formulated vaccines. Mice were immunized with 1 or 5 μg pBG505-TTT alone or were co-formulated with 10 μg pADA in the right TA muscle. Mice that were coimmunized with both pBG505-TTT and protein received 10 μg pBG505-TTT alone or with 10 μg pADA in the right TA muscle and 10 μg of recombinant BG505 protein (rBG505) co-formulated with protein adjuvants Alum (Aluminum hydroxide gel, Invivogen, Cat: vac-alu-250) or AddaVax (Invivogen, Cat: vac-adx-10) in the left TA muscle. Alum and MF59 were formulation at a 1:1 volume ratio with antigen (i.e. 65.5 μL rBG505+65.5 μL Alum or AddaVax) per the manufacturer's recommendation.

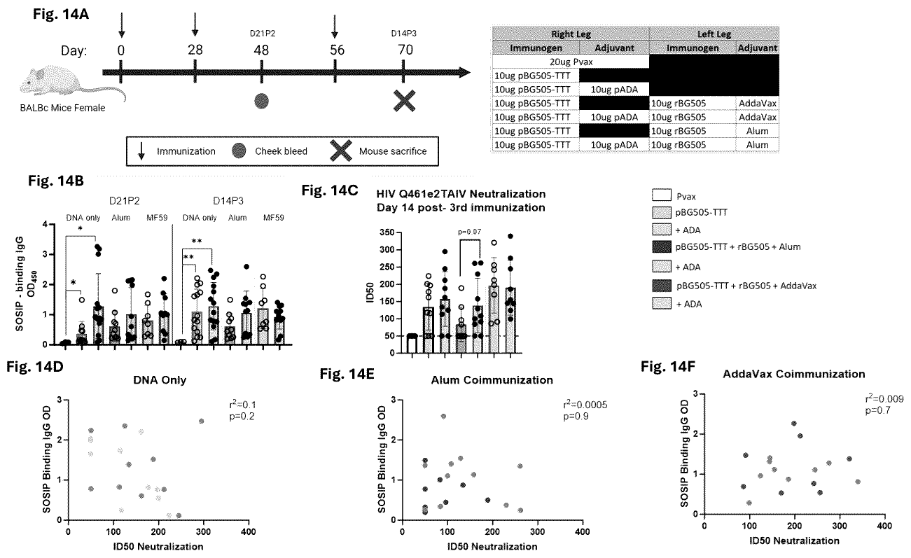

Mice that were immunized with protein alone received 10 μg rBG505 co-formulated with Alum or AddaVax in the right TA muscle (see FIG. 14). Control mice were immunized with 15 μg or 20 μg of empty plasmid vector (pVax) to ensure an equal amount of DNA was administered across experimental groups. Immediately after DNA vaccine injection, in vivo electroporation was performed using the CELLECTRA device (Inovio Pharmaceuticals, Bluebell, PA). Animals were housed in a temperature-controlled, light-cycled, specific-pathogen-free facility at Drexel University College of Medicine. Previous studies immunizing male and female mice with pADA did not show any sex biases nor any species (C57BL/6 or BALBc) biases.

### Mouse Sacrifice, Sample Collection, and Tissue Harvest

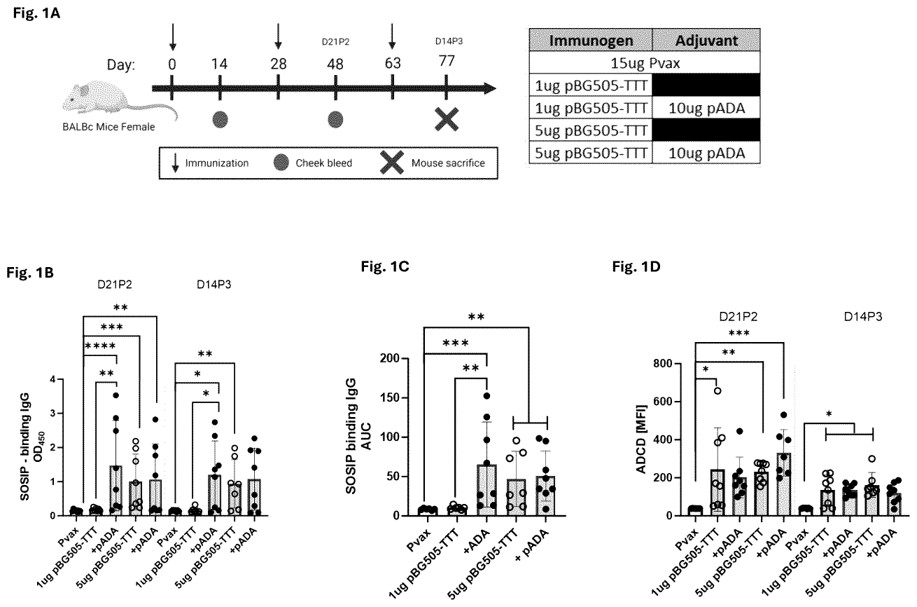

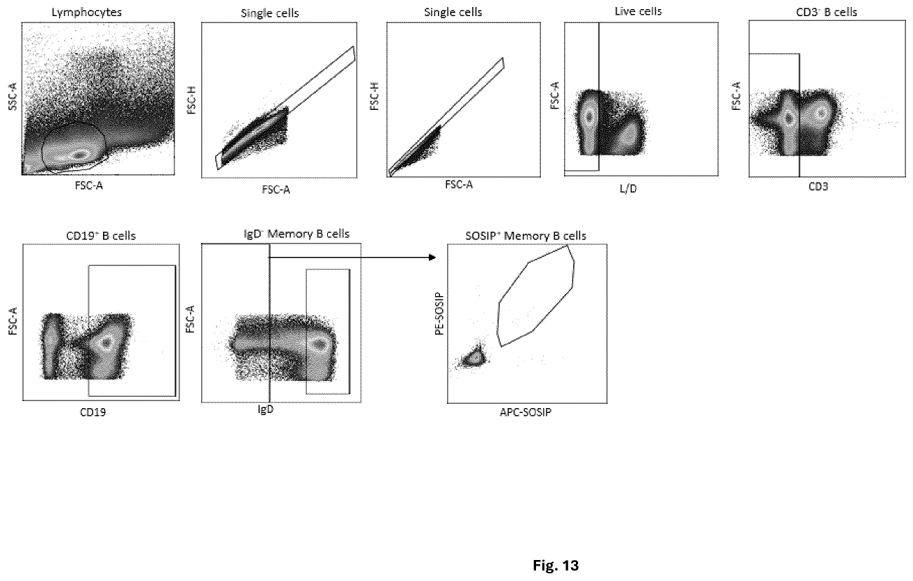

At the time points shown in the in vivo study designs (FIG. 1A and FIG. 13A), mice were either bled via cheek bleed or sacrificed. At sacrifice, blood and spleens were collected. Blood collected via cheek bleed or cardiac puncture was collected into minicollect serum gel tubes (Grenier-Bio) and centrifuged at 16,000 rpm for 10 min at 4° C. to separate serum. Separated serum was aliquoted and frozen at −80° C. for subsequent use in ELISA assays. Spleens were processed into single-cell suspensions and resuspended in RPMI medium supplemented with 1% penicillin/streptomycin and 10% FBS. Cell concentrations and viabilities were determined using a Countess Automated Cell Counter (Invitrogen, Life Technologies).

### ELISA Assays

ELISA was used to determine SOSIP-specific IgG, IgG1, IgG2a, IgG2b and IgG3 present in mouse serum. Mouse blood samples were collected via cheek bleed or cardiac puncture. Ni-NTA HisSorb plates (Qiagen, Cat: 35061) were coated with 100 μl/well recombinant his-tagged SOSIP protein that was diluted in 1×TBS (Sera Care or Fisher) to a concentration of 1-2 ug/ml and incubated at room temperature for 2 hrs. Plates were washed with 1×TBS before the addition of diluted mouse serum. For IgG and IgG1 ELISAs, mouse sera were diluted 1:100 in 1×TBS with 2% non-fat dry milk (Rockland Immunochemicals). For IgG2a, IgG2b and IgG3 ELISAs, mouse sera were diluted 1:25 in 1×TBS with 2% non-fat dry milk. Diluted serum was incubated at room temperature for 2 hrs.

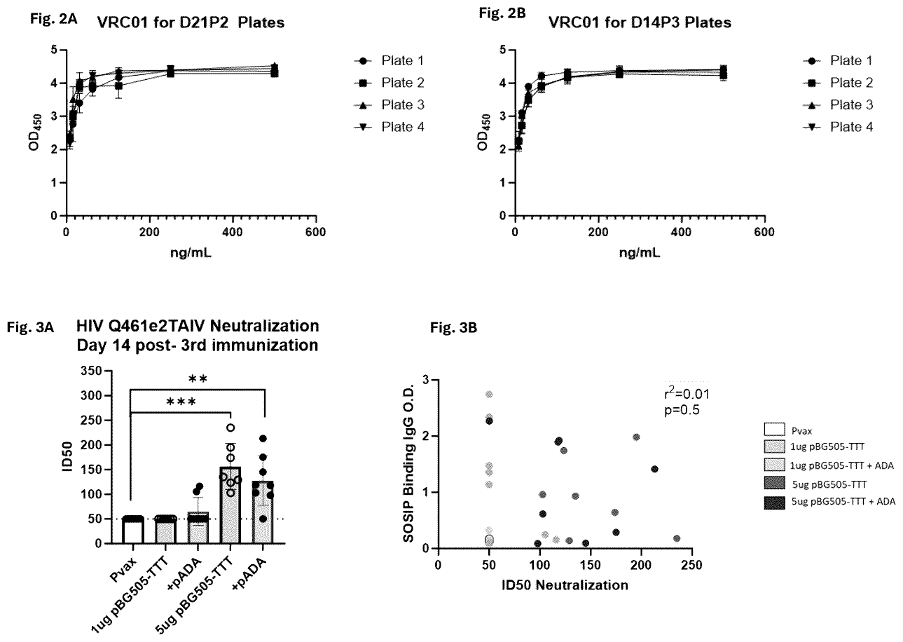

To ensure consistency from assay to assay, the human monoclonal Ab VRC01 (Cat: ARP-12033, NIH HIV reagent program) was added at a concentration of 500 ng/mL and serially diluted with 1×TBS with 2% non-fat dry milk to 7.8 ng/mL (see FIG. 2A-2B). Plates were washed with 1×TBS prior to incubation with HRP-conjugated secondary antibodies. HRP-conjugated goat anti-mouse secondary antibodies were diluted as follows in 1×TBS with 2% non-fat dry milk: IgG (Columbia Biosciences, Cat: HRP-112) 1:500, IgG1 (Invitrogen, Cat: A10551) 1:2000, IgG2a (Southern Biotech, Cat: 1081-05) 1:4000, IgG2b (Southern Biotech, Cat: 1091-05) 1:4000, and IgG3 (Southern Biotech, Cat: 1101-05) 1:4000. HRP-conjugated goat-anti human IgG (Sigma, Cat: A0293-1ML) diluted 1:3000 in 1×TBS with 2% non-fat dry milk was added to VRC01 containing wells. RP-conjugated secondary antibodies were added 100 μl/well and incubated at room temperature for 1 hr. Plates were washed with 1×TBS with 0.05% Tween-20 and developed using TMB Ultra substrate (Thermo Fisher) according to the manufacturer's instructions. SOSIP specific antibody is represented as the average O.D. at 450 nm across duplicate wells.

### Area Under the Curve Analysis

For each mouse, area under the curve (AUC) was determined for SOSIP specific IgG across the following timepoints: day 14 post 1 immunization, day 21 post 2 immunizations, and day 14 post 3 immunizations. Outliers were determined and removed using the robust regression and outlier removal algorithm in GraphPad Prism. AUC was calculated using the AUC analysis function in GraphPad Prism. Statistical significances (p<0.05) between different experimental group AUC values were determined by nonparametric Mann-Whitney U-test.

### Activation Induced Marker Assay

Splenocytes from vaccinated mice were cultured at 37° C. with 5% CO2 for 18 hrs in the presence of a megapool of BG505 SOSIP specific peptides at a concentration of 2.5 μg/mL per peptide (Cat: ARP-13123, NIH HIV reagent program) in 96-well U-bottom plates at 1×10⁶ splenocytes per well. A stimulation condition with an equal percentage amount of DMSO was included as a negative control, while PMA/Ionomycin (eBioscience, Cat: 00-4978-93) was used as a positive control. Following stimulation, cells were stained with live/dead Fixable Aqua Dead Cell Kit (Cat: L34957; Invitrogen) and anti-mouse CD16/32 (Clone: 93; Cat: 101319; BioLegend) for 20 mins at 4° cprotected from light. Cells were then surface stained for 45 mins at 4° C. protected from light with the following fluorochrome-conjugated anti-mouse Abs: CD3 (Clone: 17A2, Cat: 100216), CD4 (Clone: GK1.5; Cat: 100414), CD8 (Biolegend, Clone: 53-6.7, Cat: 100748), CD25 (Clone: PC61; Cat: 102010), OX40 (Clone: OX-86; Cat: 119411), and PD-L1 (Clone: 10F.9G2; Cat: 124321) from BioLegend. Samples were acquired on a BD LSR Fortessa and data were analyzed using FlowJo software v.10 (BD Life Sciences).

### Memory SOSIP-Specific B Cell Flow Cytometry

Biotinylated BG505 protein was incubated on ice at a concentration of 0.2 μg/μL separately with either APC-streptavidin (Biolegend, Cat: 405207) or PE-streptavidin (Biolegend, Cat: 405204) at a concentration of 0.024 μg/μL for 1 hr shielded from light. Following 1 hr incubation, 100 uM of free biotin (Invitrogen, Cat: B20656) was added and allowed to incubate for 15 mins shielded from light at 4° C. Biotinylated BG505 protein incubated with PE-streptavidin was combined with biotinylated BG505 protein incubated with APC-streptavidin at a 1:1 ratio. Single-cell suspensions of splenocytes from vaccinated mice were added to 96-well V-bottom plates at 1×10⁶ splenocytes per well. Cells were stained for viability in the presence of Live/Dead Aqua (Invitrogen, Cat: L34957) and anti-CD16/CD32 (BioLegend, Clone: 93; Cat: 101319) for 20 mins at 4° C. Cells were then washed and stained for 30 mins at 4° C. with 5 μL of the biotinylated BG505 protein probe and 95 μL of a surface antibody cocktail containing the following fluorochrome-conjugated anti-mouse Abs: CD3 (Biolegend, Clone: 17A2, Cat: 100216), CD19 (Biolegend, Clone: 6D5; Cat: 115530), and IgD (Biolegend, Clone: 11-26c.2a; Cat: 405742). Samples were acquired on a BD LSR Fortessa and data were analyzed using FlowJo software v.10 (BD Life Sciences).

### T Cell Cytokine Flow Cytometry

Splenocytes from vaccinated mice were cultured at 37° C. with 5% CO2 for 6 hrs in the presence of a megapool of BG505 SOSIP specific peptides at a concentration of 2.5 μg/mL per peptide (NIH HIV reagent program, Cat: ARP-13123) and fluorochrome-conjugated anti-CD107a (Biolegend, Clone: 1D4B, Cat: 121606) in 96-well U-bottom plates at 1×10⁶ splenocytes per well. A stimulation with an equal percentage amount of DMSO was performed as a negative control, while PMA/Ionomycin (eBioscience, Cat: 00-4978-93) was included as a positive control. During stimulation, cells were also incubated with protein transport inhibitor (ebioscience, Cat: 00-4980-93). Following stimulation, cells were stained with Live/Dead Aqua Fixable Viability Stain (Invitrogen, Cat: L34957) and anti-mouse CD16/32 (BioLegend, Clone: 93; Cat: 101319) for 20 mins at 4° C. protected from light. Cells were then surface stained for 45 mins at 4° C. protected from light with the following fluorochrome-conjugated anti-mouse Abs: CD3 (Biolegend, Clone: 17A2, Cat: 100216), CD4 (Biolegend, Clone: GK1.5; Cat: 100414), CD8 (Biolegend, Clone: 53-6.7, Cat: 100748), CD62L (Biolegend, Clone: MEL-14, Cat: 104448), and CD44 (Biolegend, Clone: IM7, Cat: 103032). Following surface staining, cells were washed and resuspended in 100 μL BD CytoFix/CytoPerm™ solution (Cat: 554722) for 1 hr at 4° C. protected from light. Cells were intracellularly stained with the following fluorochrome-conjugated anti-mouse Abs diluted in 1× perm/wash buffer (BD, Cat: 554723) for 1 hr at 4° C. protected from light: IFNγ (Biolegend, Clone: XMG1.2; Cat: 505810), TNFα (Biolegend, Clone: MP6-XT22, Cat: 506333) and IL-2 (BD, Clone: JES6-5H4; Cat: 503832). Samples were acquired on a BD LSR Fortessa and data were analyzed using FlowJo software v.10 (BD Life Sciences).

### ELISpot Assay

PVDF membrane ELISpot plates (Mabtech, Cat: 3654WP10) were activated with 35% ethanol and washed with PBS prior to coating. Plates were coated with mouse anti-IFN-7 capture antibody (R&D, Cat: SEL485) and incubated overnight at 4° C. Plates were washed with PBS and blocked for 2 hr at room temperature with PBS containing 1% BSA and 5% sucrose. Splenocytes from vaccinated mice were added (2×10⁵ cells/well) and stimulated with five BG505 SOSIP-specific peptide pools at a concentration of 2.5 μg/mL per peptide (Cat: ARP-13123, NIH HIV reagent program). A stimulation with an equal percentage amount of DMSO was performed as a negative control, while PMA/Ionomycin (ebioscience, Cat: 00-4970-93) was included as a positive control. Cells were stimulated overnight at 37° C. with 5% CO2. Plates were then washed and incubated with mouse IFN-7 detection antibody (R&D, Cat: SEL485) overnight at 4° C. Plates were washed and incubated with Streptavidin-ALP antibody for 2 hrs at room temperature prior to development. To develop, plates were incubated with BCIP/NBT substrate for 30 mins at room temperature protected from light. Plates were washed and left to dry overnight at room temperature. Plates were then scanned and counted using CTL ImmunoSpot S5 Core Analyzer (Cellular Technology Limited CTL).

### Neutralization Assay

Pseudoviruses were produced using the pSG3ΔEnv DNA plasmid encoding the HIV backbone and a plasmid encoding (Q461e2TAIV) envelope as previously described [63]. Briefly, 293T cells were plated at 5×10⁶ cells/flask in a T-75 cm flask in medium (DMEM, 10% fetal calf serum, 1% 1-glutamine, 1% penicillin-streptomycin). The following day, 293T cells in DMEM without pen/strep were transfected with 10 g total DNA/flask at a 20:1 ratio of backbone-to-Env plasmids in the presence of 20 μL polyethylenimine (jetPEI; Polyplus, Inc.) per well. Cells were incubated for 48 hr at 37° C. Virus was harvested by the centrifugation of the supernatant at 1,800 rpm for 10 min at 4° C. The pseudovirus stock was aliquoted and stored at −80° C. until use. The pseudovirus was titrated on TZM-bl cells; dilutions of the pseudovirus were used to infect TZM-bl cells in the presence of 7.5 g/ml DEAE-dextran (Fisher Biotech, Fair Lawn, NJ). Two days later, infection was measured by luciferase expression, and 50% tissue culture infective doses (TCID50) (determined by the Reed-Muench formula) were added to each neutralization assay.

The neutralization assays were performed in TZM-bl cells which have an HIV Trans-activator of Transcription (tat) induced luciferase reporter. A 3-fold dilution of the mouse serum was incubated with the tier 1A virus Q461e2TAIV for Ihr at 37° C. TZM-bl cells were added with DEAE-Dextran and allowed to incubate for 48 hrs at 37° C. with 5% CO2. Cells were lvsed with Bright-Glo (Promega) for 2 mins before being read in a luminometer. ID₅₀'s were calculated as a 50% reduction in the relative light units (RLU) in the mouse serum dilution wells as compared to the virus only wells after subtraction of the cell only controls. All samples were tested in duplicate, and response curves were fit by nonlinear regression.

### Antibody Dependent Cellular Cytotoxicity (ADCD) Assay

Antibody-dependent complement deposition (ADCD) was assessed as described previously[64]. Briefly, biotinylated BG505 protein was coupled to fluorescence NeutrAvidin beads (Thermo Fisher Scientific). Mouse sera antibodies were diluted 1:10 in 0.1% BSA and incubated with the coupled BG505 beads for 2 hrs at 37° C. Beads were washed and incubated with complement factors from guinea pig (Sigma-Aldrich) for 20 mins at 37° C. The complement reaction was then stopped by washing with 15 mM EDTA in PBS. C3 deposition on the beads was detected with a 1:100 diluted FITC-conjugated anti-guinea pig C3 polyclonal antibody (MP Biomedicals, Cat: 0855385) and relative C3 deposition was analyzed by flow cytometry.

### Statistical Analysis

All statistics were analyzed using GraphPad Prism 9 or 10. Error bars represent means a standard deviations. Outliers were determined and removed using the robust regression and outlier removal (ROUT) algorithm. Mann-Whitney U test or Kruskal-Wallis ANOVA statistical tests were performed where denotated to determine statistical differences between groups. In all data, *p<005, **p<0.01, ***p<0.001, and ****p<0.0001.

### Results

### Ada Enhances HIV Specific IgG and Antibody Dependent Complement Deposition (ADCD)

To evaluate the effect of ADA on HIV specific humoral responses, 6-8-week-old female BALB/c mice were immunized thrice with 1 μg pBG505-TTT or 5 μg pBG505-TTT alone or in combination with 10 g pADA. Control mice were immunized with 15 μg Pvax, an empty plasmid vector, to ensure mice received equal total amounts of DNA and to control for any nonspecific DNA-induced innate immune responses. Mice were bled on day 14 post 1 immunization (D14P1), day 21 post 2 immunizations (D21P2), and sacrificed on day 14 post 3 immunizations (D14P3) to evaluate SOSIP specific antibody magnitude, durability, and function via ELISA, antibody dependent complement deposition (ADCD) or neutralization assays (FIG. 1A). Consistency between ELISA assays was confirmed using the human monoclonal antibody VRC01 (FIG. 2A-2B).

Mice co-immunized with pADA and 1 μg pBG505-TTT exhibited significantly increased SOSIP specific IgG (mean O.D.=1.482) compared to mice immunized with 1 μg pBG505-TTT alone (mean O.D.=0.184) at both D21P2 (**p=0.0022) and D14P3 (*p=0.0289). Mice co-immunized with pADA and 5 μg pBG505-TTT however, exhibited comparable levels of SOSIP specific IgG (mean O.D.=1.063) to mice immunized with 5 μg pBG505-TTT alone (mean O.D.=0.998) suggesting a plateau in the humoral response with 5 μg pBG505-TTT. The ability of pADA to significantly enhance humoral responses with 1 μg pBG505-TTT (mean O.D.=1.482) to levels comparable with 5 μg pBG505-TTT (mean O.D.=0.998) clearly demonstrates a dose sparing effect with pADA (FIG. 1B). To evaluate the durability of the SOSIP specific IgG induced following immunization, an area under the curve (AUC) analysis was performed on SOSIP specific IgG that was measured across all timepoints (D14P1, D21P2, and D14P3). Mice co-immunized with pADA and 1 μg pBG505-TTT (mean AUC=65.47) exhibited significantly increased SOSIP specific IgG over time (**p=0.0027) compared to mice immunized with 1 μg pBG505-TTT alone (mean AUC=10.36) suggesting that ADA induces more durable humoral responses (FIG. 1C).

To evaluate the functionality of SOSIP specific Ab, we performed an antibody dependent complement deposition (ADCD) assay using sera from immunized mice at D21P2 and D14P3. At the D21P2 timepoint, mice co-immunized with pADA and 5 μg pBG505-TTT produced SOSIP specific Ab with the most capacity to induce ADCD (mean MFI=331.14) with a trend toward increased ADCD compared to mice that received 5 μg pBG505-TTT alone (mean MFI=232.38), although not significant. Mice co-immunized with 1 μg pBG505-TTT and pADA (mean MFI=201.48) did not exhibit increased ADCD compared to mice immunized with 1 μg pBG505-TTT alone (mean MFI=243.39). Compared to Pvax immunized mice, mice immunized with 1 μg pBG505-TTT (*p=0.0307), 5 μg pBG505-TTT (**p=0.0038) and 5 μg pBG505-TTT with pADA (***p=0.0001) had significantly increased ADCD. This suggests the ability of ADA to increase ADCD particularly when co-administered with 5 μg pBG505-TTT. At the D14P3 timepoint, ADCD capacity contracts across all immunization groups compared to the D21P2 timepoint and there is no difference between mice immunized with pBG505-TTT alone or co-immunized with pADA (FIG. 1D).

In addition to ADCD, Ab neutralization capacity was evaluated. Mice immunized 5 μg pBG505-TTT alone (***p=0.0006, mean ID50=156.29) and with pADA (**p=0.0014, mean ID50=127.59) had significantly increased neutralization against the tier 1A virus Q461e2TAIV compared to Pvax mice (FIG. 3A). Additionally, we observed no correlation between SOSIP specific IgG magnitude and neutralization (FIG. 3B).

These data demonstrate that pADA is dose sparing, enhances SOSIP specific IgG magnitude and durability as well as increases the ability of SOSIP specific antibody to induce ADCD.

### Ada Enhances HIV Specific Antibody Isotype Switching

To evaluate if ADA enhances isotype switching, we measured SOSIP specific IgG1, IgG2a, IgG2b and IgG3 in the sera of immunized mice as shown in FIG. 1A.

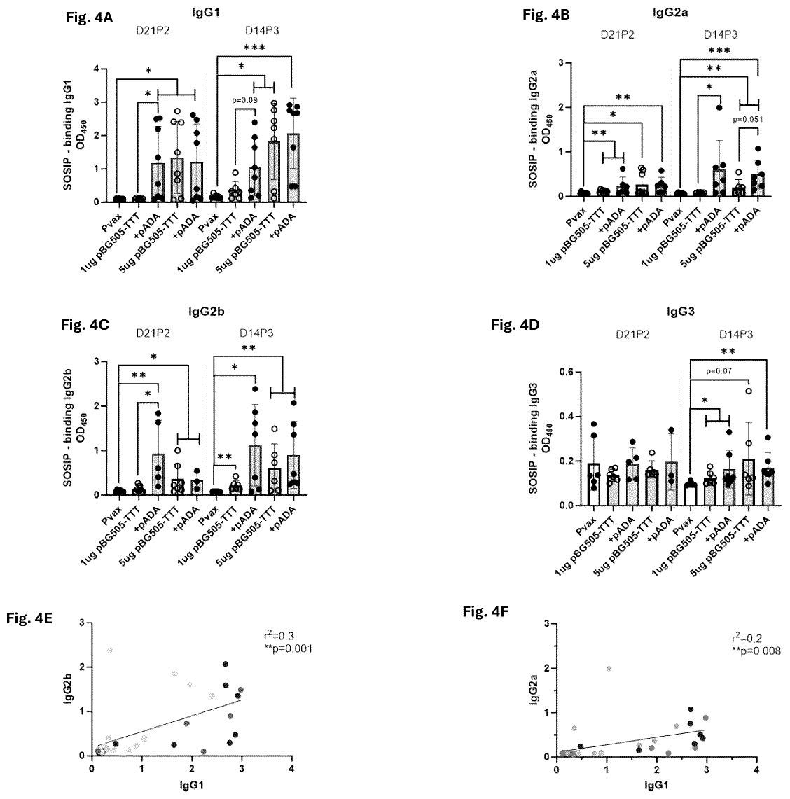

Mice co-immunized with pADA and 1 μg pBG505-TTT exhibited significantly increased SOSIP specific IgG1 at D21P2 (*p=0.0137, mean O.D.=1.186) and a near significant increase (p=0.0939, mean O.D.=1.057) at D14P3 compared to mice immunized with 1 μg pBG505-TTT alone (mean O.D. D21P2=0.104, mean O.D. D14P3=0.348). Like total SOSIP specific IgG, SOSIP specific IgG1 levels plateau at 5 μg pBG505-TTT (mean O.D. D21P2=1.341, mean O.D. D14P3=1.821) and we observe no further enhancement with pADA (mean O.D. D21P2=1.208, mean O.D. D14P3=2.059) (FIG. 4A).

At D21P2 we observed little to no SOSIP specific IgG2a in both pADA receiving (mean O.D. 1 μg pBG505-TTT+pADA=0.230, mean O.D. 5 μg pBG505-TTT+pADA=0.255) and non-receiving counterparts (mean O.D. 1 μg pBG505-TTT=0.113, mean O.D. 5 μg pBG505-TTT=0.269). However, at D14P3 we see that only mice that were co-immunized with pADA develop SOSIP specific IgG2a (mean O.D. 1 μg pBG505-TTT+pADA=0.594, mean O.D. 5 μg pBG505-TTT+pADA=0.493). The increase in SOSIP specific IgG2a in pADA co-immunized mice was significant when compared to mice receiving 1 μg pBG505-TTT alone (*p=0.0192) and near significant when compared to mice receiving 5 μg pBG505-TTT alone (p=0.0513). These data demonstrate the ability of ADA to uniquely induce SOSIP specific IgG2a which is not achieved with pBG505-TTT alone (FIG. 4B). When correlating SOSIP specific IgG1 to IgG2a production, we observe a significant positive correlation (**p=0.008) between SOSIP specific IgG1 and IgG2a (FIG. 4F).

Mice co-immunized with pADA and 1 μg pBG505-TTT exhibited significantly increased SOSIP specific IgG2b at D21P2 (*p=0.0101, mean O.D.=0.933) and a trend of increased SOSIP IgG2b (mean O.D.=1.121) at D14P3 compared to mice immunized with 1 μg pBG505-TTT alone (mean O.D. D21P2=0.142, mean O.D. D14P3=0.221). Similar to SOSIP specific IgG and IgG1 responses, SOSIP specific IgG2b levels plateau at 5 μg pBG505-TTT (mean O.D. D21P2=0.360, mean O.D. D14P3=0.608) and we observe no further enhancement with pADA (mean O.D. D21P2=0.332, mean O.D. D14P3=0.900) (FIG. 4C). When correlating SOSIP specific IgG1 to and IgG2b production, we observe a significant positive correlation (**p=0.001) between SOSIP specific IgG1 and IgG2b (FIG. 4E). Regardless of timepoint or immunization, we observed minimal SOSIP specific IgG3 production with no differences between mice immunized with pBG505-TTT alone or with pADA (FIG. 4D).

These data demonstrate that pADA enhances the isotype switching of SOSIP specific Abs and uniquely drives IgG2a expression.

### Ada Enhances HIV Specific T Cell Responses

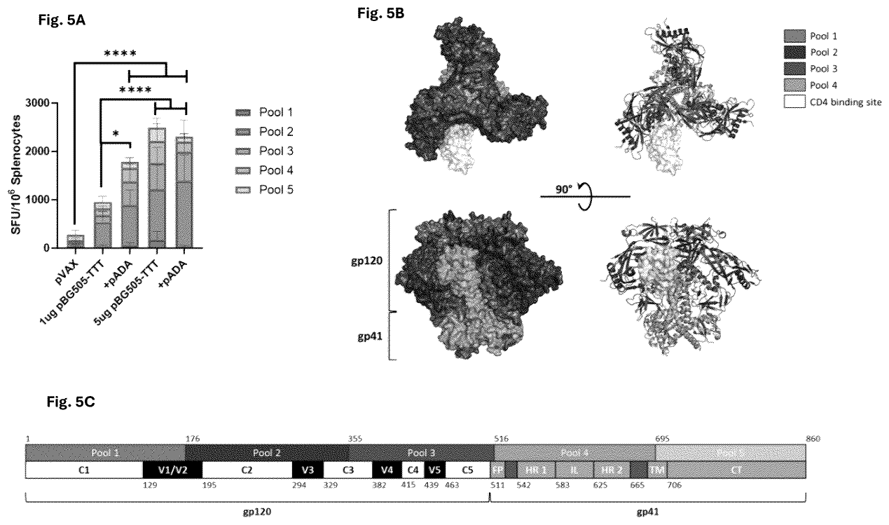

To quantify HIV-BG505 cell mediated immunity, we performed an IFNγ ELISpot assay using splenocytes from immunized mice as outlined in FIG. 1A. Spleens were harvested D14P3 and stimulated with peptide pools encompassing the HIV BG505 protein. HIV specific IFNγ production was quantified by spot forming units (SFUs) per million splenocytes using an ELISpot assay. In line with the humoral responses (FIGS. 1 and 4), mice co-immunized with pADA and 1 μg pBG505-TTT had significantly increased IFNγ SFUs (*p=0.0455, mean SFU=1785) compared to mice with 1 μg pBG505-TTT alone (mean SFU=958). Similarly, humoral response findings, we observed a plateau in SOSIP specific IFNγ production in mice immunized with 5 μg pBG505-TTT alone (mean SFU=2496) or with pADA (mean SFU=2312) (FIG. 5A). These data suggest that ADA co-delivery can enhance SOSIP specific cellular IFN7 production and humoral responses (FIG. 1) in a dose sparing manner.

To understand immunogenic epitopes driving T cell responses, we colored the BG505 peptide pools onto the corresponding regions of the BG505 protein using a cryo-EM structure of a BG505 SOSIP.664 trimer (PDB ID: 6V0R). To determine the CD4 binding site, a cryo-EM structure of gp140 bound to the extracellular domains of CD4 (PDB ID: 7T0O) was aligned to the BG505 SOSIP.664 trimer. T cell responses against pBG505-TTT were primarily driven by the BG505 peptide pool 2 and pool 3. Pool 2 and 3 spanned the majority of the gp120 subunit. Notably, these peptide pools spanned important epitopes of BG505 including variable loops (V1/2, V3, V4 and V5) and the CD4 binding site (FIG. 5B-5C). These findings demonstrate the ability of pBG505-TTT to drive T cell responses against important epitopes of the BG505 protein.

### Ada Enhances HIV Specific T Cell Polyfunctionality and Degranulation

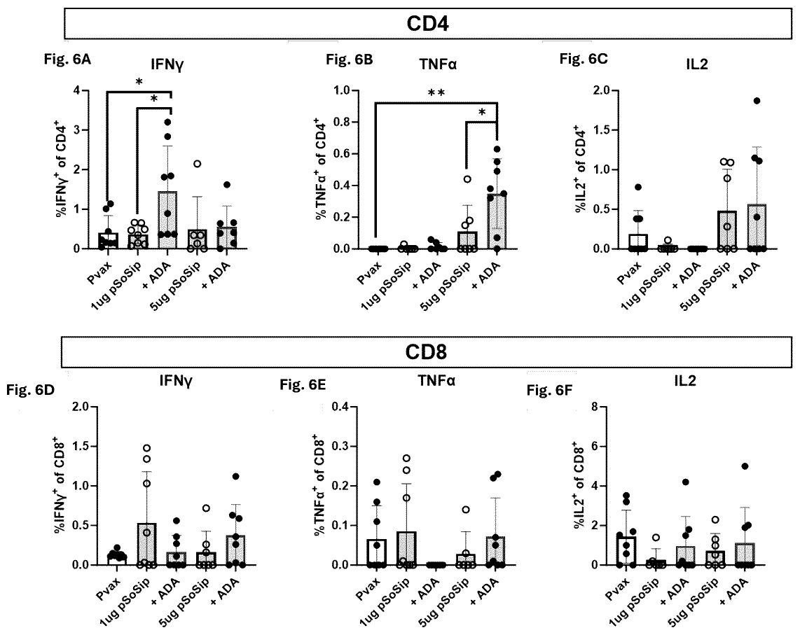

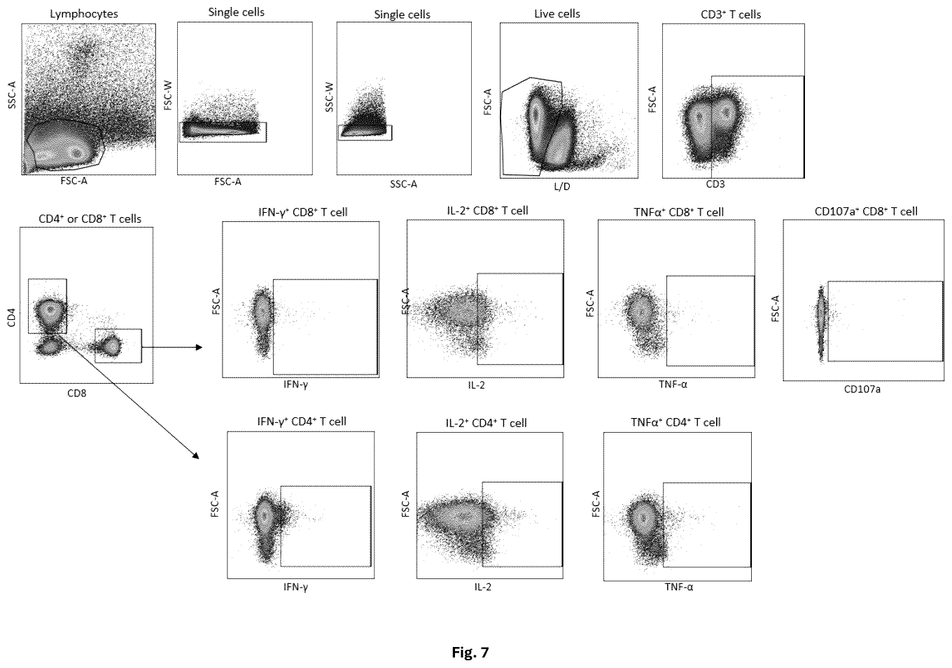

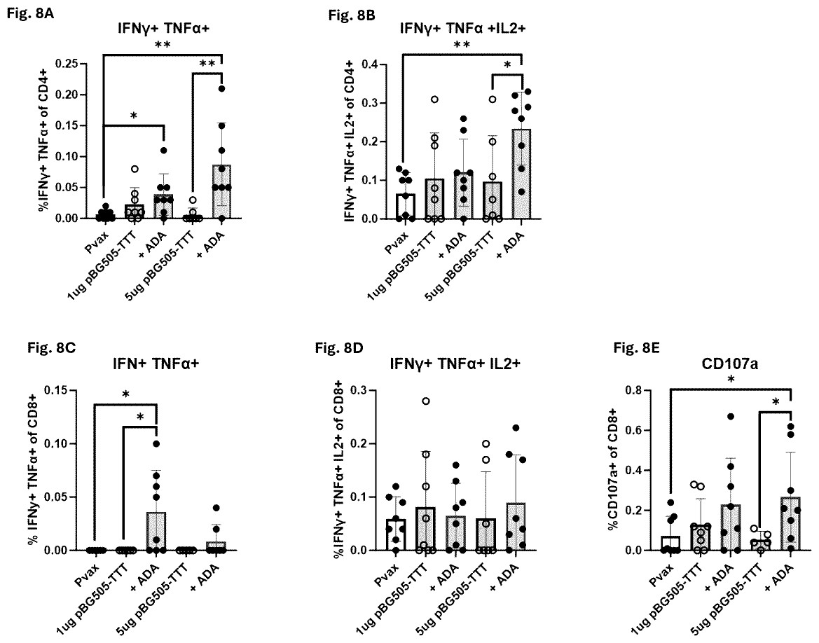

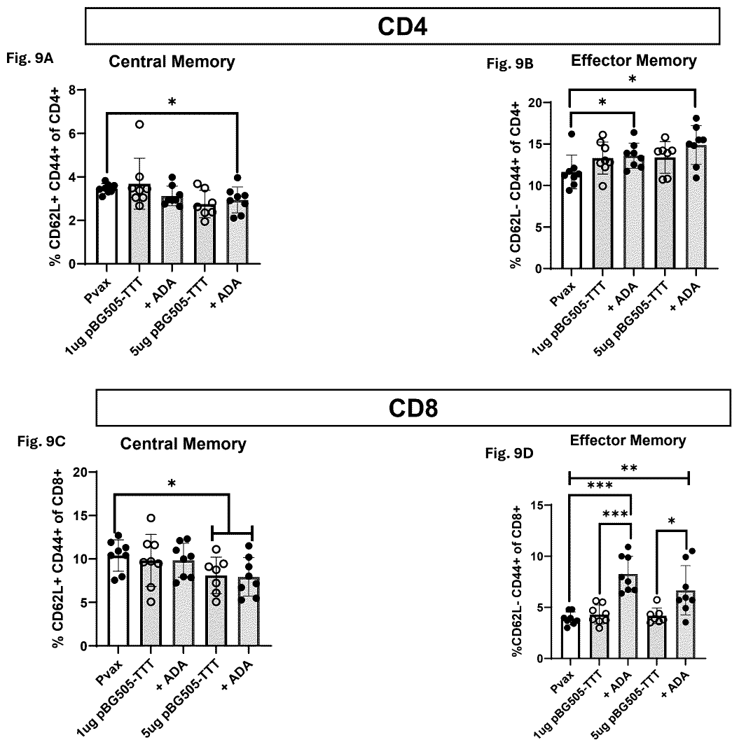

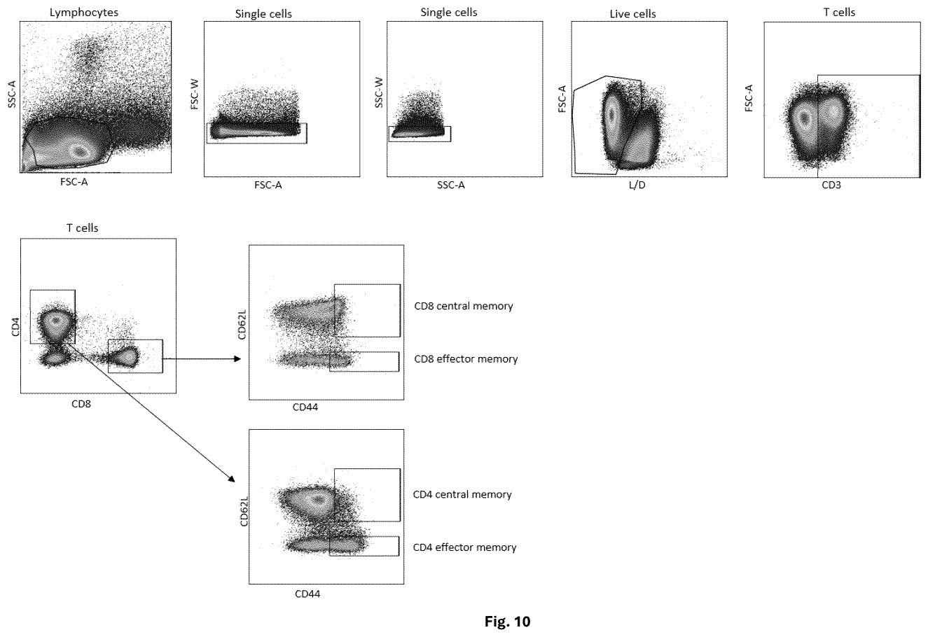

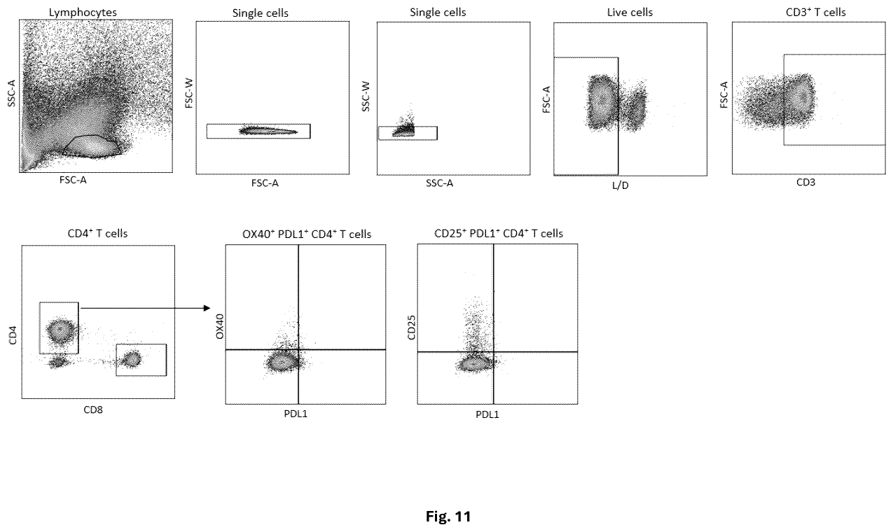

To further quantify HIV-BG505 cell mediated immunity, we performed intracellular cytokine staining (ICS) to determine monofunctionality (IFNγ, TNFα or IL-2 expression, (FIG. 6) and polyfunctionality (IFNγ and TNFα or IFNγ, TNFα and IL-2 expression) of both CD4⁺ (FIG. 8A-8B) and CD8⁺ (FIG. 8C-8D) T cells using spleenocytes from immunized mice as outlined in FIG. 1A. The gating strategy used is displayed in FIG. 7. Spleens were harvested on D14P3 and stimulated with peptide pools encompassing the HIV BG505 protein. Simultaneously, cells were stained for CD62L and CD44 to quantify central memory (CD62L⁺, CD44⁺) and effector memory (CD62L⁻, CD44⁺) T cells (FIG. 9). The gating strategy used is shown in FIG. 10. Cells were also stained for CD107a, a degranulation marker, to evaluate CD8⁺ T cell cytotoxicity (FIG. 8E).

When evaluating monofunctionality of CD4⁺ T cells, ADA co-immunization resulted in increased frequencies of CD4⁺ T cells producing IFNγ and TNFα. Mice co-immunized with pADA and 1 μg pBG505-TTT has significantly increased frequencies of IFNγ⁺ CD4⁺ T cells compared to mice immunized with 1 μg pBG505-TTT (*p=0.0351) or Pvax (*p=0.0256) (FIG. 3.6A). Mice coimmunized with pADA and 5 μg pBG505-TTT had significantly increased frequencies of TNFα⁺ CD4⁺ T cells compared to mice immunized with 5 μg pBG505-TTT (*p=0.0499) or Pvax (**p=0.0014) (FIG. 6B). We observed a trend of increased IL2⁺ CD4⁺ T cell frequencies with pADA and 5 μg pBG505-TTT, although not significant when compared to mice receiving 5 μg pBG505-TTT alone (FIG. 6C).

When evaluating polyfunctionality of CD4⁺ T cells, we found that mice co-immunized with pADA and 5 μg pBG505-TTT had significantly increased frequencies of IFNγ⁺ TNFα⁺ CD4⁺ T cells compared to mice that were immunized with Pvax (**p=0.0047) and 5 μg pBG505-TTT alone (**p=0.0030) (FIG. 8A). Mice co-immunized with pADA and 1 μg pBG505-TTT had significantly increased frequencies of IFNγ⁺ TNFα⁺ CD4⁺ T cells compared to Pvax (*p=0.0120) and an increasing trend when compared to mice immunized with 1 μg pBG505-TTT alone although not significant (FIG. 8A). Mice co-immunized with pADA and 5 μg pBG505-TTT had significantly increased frequencies of IFNγ⁺ TNFα⁺ IL2⁺ CD4⁺ T cells compared to mice that were immunized with Pvax (**p=0.0023) 5 μg pBG505-TTT alone (*p=0.0389) (FIG. 8B).

In the CD8⁺ T cell compartment, we observed no differences in monofunctionality between mice that had been co-immunized with pADA and mice immunized with pBG505 (FIG. 6D-6F). In terms of CD8⁺ T cell polyfunctionality, we observed that mice co-immunized with pADA and 1 μg pBG505-TTT had significantly increased frequencies of IFNγ⁺ TNFα CD8⁺ T cell compared to mice immunized with Pvax (*p=0.0430) and 1 μg pBG505-TTT alone (*p=0.0430) (FIG. 8C). When evaluating CD8⁺ T cell producing IFNγ, TNFα, and IL-2, however, we observed no differences between mice coimmunized with pADA or immunized with pBG505 alone (FIG. 8D). Mice co-immunized with pADA and 5 μg pBG505-TTT had significantly increased frequencies of CD8⁺ T cells expressing the degranulation marker CD107a compared to mice immunized with Pvax (*p=0.0258) and 5 μg pBG505-TTT alone (*p=0.0420) (FIG. 8E). Enhanced CD107a expression with pADA co-immunization indicates that ADA increases the cytotoxicity [65] of CD8⁺ T cells.

When evaluating central and effector memory T cell responses, we observed no effect on CD4⁺ central memory (CD62L⁺, CD44⁺) T cells (FIG. 9A) with or without pADA. Mice co-immunized with 1 μg pBG505-TTT (*p=0.0115) or 5 μg pBG505-TTT (*p=0.0263) and pADA did have significant increases in frequencies of CD4⁺ effector memory (CD62L⁻, CD44⁺) T cells compared to Pvax immunized mice. These increases with pADA however, were not significant when compared to mice immunized with 1 μg pBG505-TTT or 5 μg pBG505-TTT alone (FIG. 9B). Similar to CD4⁺ T cell central memory, we observed no effect on CD8⁺ T cell central memory (CD62L⁺, CD44⁺) frequencies (FIG. 9C) with or without pADA. In contrast to CD8⁺ T cell central memory, we observed a significant increase in frequencies of CD8⁺ effector memory (CD62L⁻, CD44⁺) T cells with pADA communization at both the 1 μg pBG505-TTT (***p=0.0002) and 5 μg pBG505-TTT (*p=0.0289) doses compared to mice immunized with 1 μg pBG505-TTT or 5 μg pBG505-TTT alone (FIG. 9D). Frequencies of CD8⁺ effector memory (CD62L⁻, CD44⁺) T cells were also significantly increased in mice immunized with 1 μg pBG505-TTT and pADA (***p=0.0002) and 5 μg pBG505-TTT+pADA (**p=0.0047) doses compared to mice immunized with Pvax.

Taken together, these data demonstrate that ADA enhances HIV specific T cell cytokine production and polyfunctionality. These data also suggest that ADA enhances CD4⁺ and CD8⁺ effector memory T cells responses but has little to no effect on central memory T cell responses.

### ADA Enhances HIV Specific T Cell Activation and Memory B Cell Frequencies

To further quantify HIV-BG505 cell mediated immunity, we performed an activation induced marker (AIM) assay and quantified SOSIP specific memory B cells using flow cytometry.

The AIM assay, as previously reported [41], is used assess antigen specific activation of CD⁺ T cells through the upregulation of activation markers such as PDL1, OX40 and CD25 upon stimulation. For these experiments, spleenocytes from immunized mice as outlined in FIG. 1A were stimulated with peptide pools encompassing the HIV BG505 protein and PDL1, OX40 and CD25 expression on CD⁺ T cells was quantified via flow cytometry. The gating strategy used is shown in FIG. 11.

To evaluate SOSIP specific memory B cell responses, we stained spleenocytes from immunized mice as outlined in FIG. 1A with fluorescently labeled multimerized probes specific to SOSIP and defined memory B cells as spleenocytes that were CD19⁺IgD⁻ via. The corresponding flow cytometry gating scheme is displayed in FIG. 13.

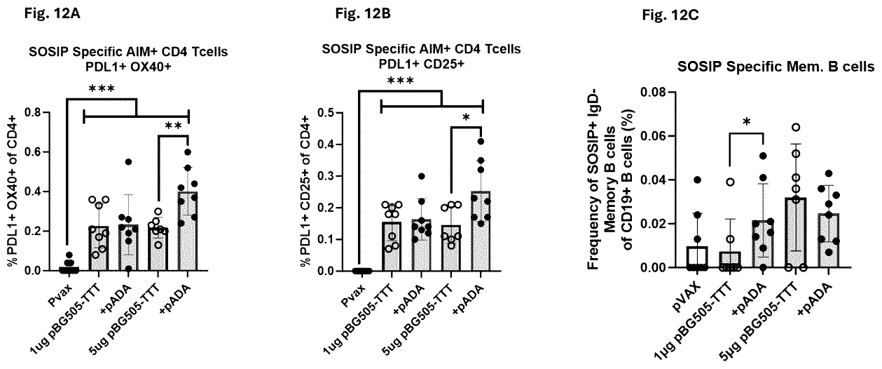

Mice co-immunized with pADA and 5 μg pBG505-TTT had significantly increased frequencies of CD4⁺ T cells expressing PDL1 and OX40 (**p=0.0022, mean=0.401%) compared to mice immunized with 5 μg pBG505-TTT alone (mean=0.217%) (FIG. 12A). Mice co-immunized with pADA and 5 μg pBG505-TTT also had significantly increased frequencies of CD4⁺ T cells expressing PDL1 and CD25 (*p=0.0280, mean=0.253%) (FIG. 12B) compared to mice immunized with 5 μg pBG505-TTT alone (mean=0.146%).

In line with the humoral response (FIGS. 1 and 4), mice co-immunized with pADA and 1 μg pBG505-TTT had significantly increased frequencies of SOSIP specific memory B cells (*p=0.0382, mean=0.022%) compared to mice immunized with 1 μg pBG505-TTT alone (mean=0.007%). At the 5 μg pBG505-TTT dose, we observed no difference in SOSIP specific memory B cell frequencies in mice co-immunized with pADA (mean=0.025%) or immunized with 5 μg pBG505-TTT alone (mean=0.032%) (FIG. 12C).

Taken together, these data demonstrate that ADA enhances HIV specific CD4⁺ T activation and SOSIP specific memory B cells.

### ADA Enhances Neutralization Capacity of HIV Specific Antibodies

To see any qualitative difference in Ab responses within a BALB/c mouse model, immunization with DNA, protein and an adjuvant is necessary. To address this, we immunized mice simultaneously with 10 g pBG505-TTT alone or with pADA in the right tibialis anterior (TA) muscle and 10 μg rBG505 with alum or with the MF59-like adjuvant AddaVax in the left TA muscle. To understand how pADA performs compared to Alum and AddaVax, we immunized a separate group of mice with 10 μg pBG505-TTT alone or with pADA and no protein co-immunization. As a negative control, we immunized mice with 20 μg of the empty plasmid vector Pvax. Mice were immunized thrice and bled on D21P2 and sacrificed at D14P3 (FIG. 14A).

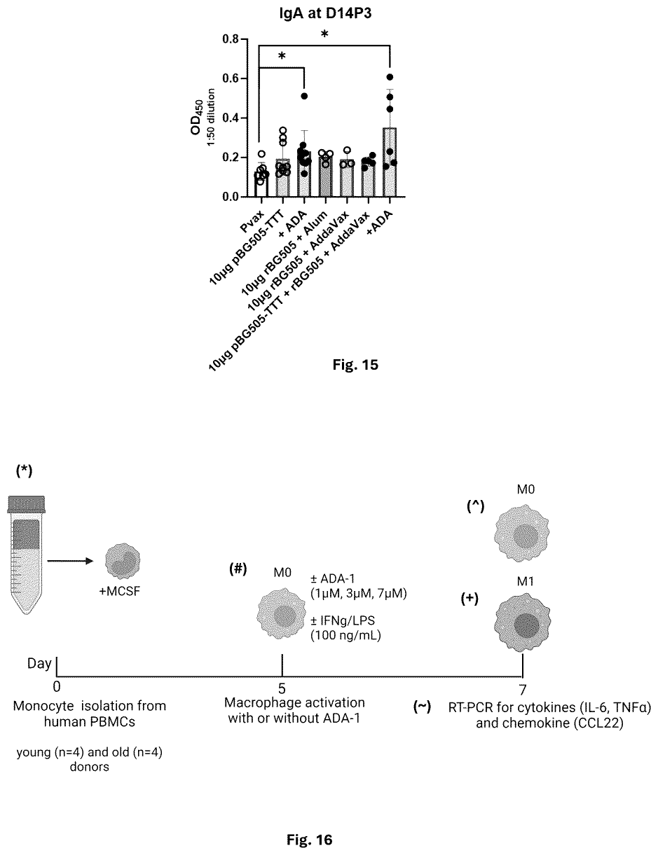

When evaluating SOSIP specific IgG, across all immunization groups we observed no difference between mice that received pADA or their non-pADA receiving counterparts (FIG. 14B). This is likely due to the higher dose of pBG505-TTT (10 μg) used for this experiment as we observed a plateau in humoral responses with 5 μg pBG505-TTT (FIGS. 1 and 4). We also evaluated SOSIP specific IgA responses and observed no difference between mice that received pADA or their non-pADA receiving counterparts. We did, however, observe a significant increase in SOSIP specific IgA in mice immunized with pBG505-TTT and pADA (*p=0.0307) and mice immunized with pBG505, rBG505, Addavax and pADA (*p=0.0121) compared to Pvax immunized mice. There was an observed trend toward increased SOSIP specific IgA in pADA co-immunized mice compared to their non-ADA receiving counterparts although not significant (FIG. 15).

When evaluating Ab functionality by live viral neutralization assay, ADA did increase neutralization of the tier 1A virus Q461eTAIV to a near significant level (p=0.07) compared to mice immunized with pBG505-TTT, rBG505 and alum. Mice co-immunized with pBG505-TTT, rBG505 and AddaVax had robust neutralization that was not further improved with pADA (FIG. 14C). We observe no correlation between SOSIP specific IgG and neutralization ID50s demonstrating that the increased Ab quality with pADA is not a direct result of increased Ab magnitude (FIG. 14D-14F).

Collectively these data demonstrate the ability of ADA to enhance the quality of SOSIP-specific Abs by improving neutralization capacity.

### Discussion

We have shown that ADA-1 is critical to the T follicular helper (TFH) program and that the addition of exogenous ADA-1 enhanced the ability of less efficient pre-TFH to provide help to B cells [27]. We believe ADA-1 is therefore unique as an immune modulator as it targets TFH cells, and TFH cells are central to many aspects critical for effective anti-HIV humoral and cellular responses. TFH cells are known to be critical orchestrators of germinal center reactions which are needed for long-lived humoral and cellular responses. TFH cells are critical to several processes, such as isotype switching, somatic hypermutation, and affinity maturation in B cells, which in turn are needed for the development of bNAbs and therefore critical for an effective anti-HIV humoral response [66]. TFH cells have also been found to boost CD8⁺ T function and enhanced CD8⁺ T function has been implicated in enhanced protection against HIV [57-59]. We therefore hypothesized that ADA-1 combined with pBG505-TTT would result in enhanced magnitude and quality of humoral and cellular HIV specific responses. Our findings demonstrate that ADA-1 has a strong impact on both cellular and humoral immune responses particularly CD8⁺ T cells and qualitative Ab responses which are important contributors to anti-HIV immune responses.

Herein, we demonstrated that pADA co-immunization with pBG505-TTT enhances HIV specific humoral and cellular responses. In terms of humoral responses, we observed that pADA co-immunization increases SOSIP-specific Ab magnitude, durability, and isotype switching. This supports our previous findings of pADA enhancing humoral responses to SARS-CoV-2 [31, 32] and monomeric gp160 HIV envelope [25] DNA vaccines. ADA-1 co-immunized mice exhibited an increase in ADCD thereby inducing the nNAbs that are mediating this FcγR-mediated Ab function. ADA-1 is therefore driving ADCD which has been shown to decrease HIV replication and may play a role in HIV protection [49-54].

ADA-1 induced robust SOSIP-specific IgG, IgG1, IgG2a and IgG2b antibodies at strikingly low doses of pBG505-TTT (1 μg) and in a dose-sparing manner. Studies using plasmid encoded MD39, another native-like Env immunogen, required doses of 25 μg of DNA to see improved humoral responses [45]. Herein, we found that pADA uniquely induces IgG2a production which was not achieved with pBG505-TTT alone. IgG2a production is driven by TH1 responses and, functionally, IgG2a has been correlated with influenza viral clearance and protection against challenge in mice [67] as well as increased overall efficacy of influenza vaccination [68-71], suggesting an important role of IgG2a in viral infections. When considering affinity for Fc receptors to mediate antibody dependent effector functions, mouse IgG2a seems to be a functional analog to human IgG3 [72-74]. HIV infected elite controllers have enhanced antibody functionality including ADCC, monocyte and neutrophil phagocytosis and ADCD primarily coordinated through human IgG3/IgG1 responses [75]. Therefore, the ADA-1 driven enhancement of mouse IgG2a observed contributes to enhanced ADCD observed here and indicates that ADA-1 could induce human IgG3 with enhanced functionality similar to that observed in elite controllers. Additionally, we found that ADA-1 induced a SOSIP-specific IgA response when co-immunized with pBG505, rBG505, and Addavax indicating that ADA-1 can induce mucosal immunity critical for protection against HIV infection.

Also demonstrated herein is the ability of ADA-1 to enhance antibody quality. We observed that pADA co-immunization enhanced SOSIP-specific Ab neutralization compared to mice immunized with pBG505, rBG505 and alum. Increased neutralization capacity is a likely result of increased somatic hypermutation and antibody affinity. T Collectively these findings suggest that ADA-1 is driving key humoral responses that would be necessary for an effective HIV vaccine.

In terms of cellular responses, the data presented herein demonstrates that ADA-1 enhances HIV-specific CD4⁺ and CD8⁺ T cell responses. ADA-1 co-immunization led to increased HIV-specific IFN-7 production in splenocytes, presumably from both CD4⁺ and CD8⁺ T cells. T cell responses against pBG505-TTT were primarily driven by the BG505 peptide pool 2, which encompassed the variable loop V3 and CD4 binding site. ADA-1 co-immunization also led to increased HIV specific polyfunctionality from both CD4⁺ and CD8⁺ T cells as well as enhanced activation in CD4⁺ T cells and enhanced degranulation (i.e. CD107a expression) in CD8⁺ T cells. We also demonstrated that ADA-1 enhanced frequencies of CD8⁺ effector memory T cells. HIV elite controllers have been found to have CD8⁺ T cells with increased memory and effector potential which importantly provides an increased ability to kill HIV infected cells before progeny virions can be produced [58, 76-78]. Vaccine strategies designed to enhance CD8⁺ T cell frequency and effector function have also demonstrated protective HIV immunity in NHP [59]. Therefore, the increased CD8⁺ effector memory T cells induced by ADA-1 could contribute protective HIV immunity. In terms of B cell responses, we also demonstrate the ability of ADA-1 to enhance SOSIP-specific memory B cell responses. These findings indicate that ADA-1 is driving key cellular responses that would be necessary for an effective HIV vaccine.

The data herein demonstrated that ADA-1 can act as an immune modulator to enhance HIV-specific humoral and cellular responses in the context of a BG505-SOSIP DNA vaccine. Notably, enhanced humoral and cellular responses with ADA-1 were achieved at very low doses of pBG505-TT, exemplifying the ability of ADA-1 to be dose-sparing. This study demonstrated the ability of ADA-1, when formulated with DNA antigens, to target TFH cells and induce durable, higher-quality antibodies which are critical in the development of protective bNAbs against HIV. Importantly, ADA-1 as an enzyme replacement therapy to treat ADA-SCID has already been approved, is clinically well-tolerated and has a demonstrated safety profile [29].

### Example II: ADA-1 Enhances IL-6 and TNFα Gene Expression in Macrophages

Macrophages are key innate immune cells that drive immediate, nonspecific immune responses against pathogens. Although dendritic cells (DCs) are the major antigen presenting cells (APC) driving vaccine immune responses, macrophages are also APCs that contribute to this process. During aging there is a progressive decline in macrophage function. Adenosine deaminase 1 (ADA-1) is a ubiquitously expressed enzyme with known immunomodulatory properties.

Herein, we demonstrate that ADA-1 enhances TNFα and IL-6 gene expression in human macrophages from both young (<65 yrs) and aged donors (≥70 yrs). Notably ADA-1 rescues age associated deficits in TNFα and IL-6 gene expression. Additionally, we found that aged macrophages upregulate CCL22 expression compared to young macrophages. The data presented herein demonstrates that ADA-1 enhances the cytokine and chemokine profile young macrophages while also rescuing age associated deficits in cytokine and chemokine expression. Additionally, this data aids in elicidating the mechanism of ADA-1 as a molecular vaccine adjuvant to enhance immune responses.

Innate immune cells such as monocytes and macrophages are important for initiating immediate, nonspecific immune responses against pathogens. Monocytes serve as the free roaming cells that rapidly traffic to sites of infection and differentiate into DCs or macrophages [86-88]. Macrophages are classified into three subsets; non-activated M0 macrophages, pro-inflammatory M1 macrophages or anti-inflammatory M2 macrophages [89]. M0 macrophages are in a resting state and can be subsequently polarized to either M1 or M2 macrophages depending on the activation stimulus [89, 90]. Once activated, M1 macrophages secrete pro-inflammatory cytokines TNFα, IL-6, and IL-12 and chemokines CXCL9 and CXCL10 to recruit monocytes, helper CD4⁺ T cells and effector CD8⁺ T cells. Activated M1 macrophages also upregulate MIC-11 expression and maximize their reactive oxygen species (ROS) and nitric oxide content allowing them to kill invasive pathogens more efficiently [87, 91, 92]. M2 macrophages secrete anti-inflammatory cytokines IL-4, IL-10, and TGF-β and chemokines CCL17, CCL22, CCL24 and CXCL13 to recruit regulatory CD4⁺ T cells [92]. Macrophages function as an important bridge between innate and adaptive immunity by presenting antigen for the priming and activation of adaptive immune cells [88, 93].

During aging there is a progressive decline in the function of the immune system known as immunosenescence [88] which contributes to decreased immune responses in aged populations to several vaccines, such as influenza [94-96], hepatitis B virus [97, 98], and SARS-CoV-2 [99]. The progressive decline in immune function also increases the vulnerability of aged populations to infection and subsequent morbidity and mortality [32, 88]. Several studies have demonstrated that aging negatively impacts macrophage function. Aged macrophages [100] exhibit impaired cell signaling and reduced expression of pattern recognition receptors (PRRs) such as toll-like receptors (TLRs). This is thought to contribute to the reduced cytokine production, particularly IL-1 [101, 102], TNFα [100, 102-104] and IL-6 [100, 103] and reduced chemokine production upon LPS stimulation in aged macrophages [105]. In addition to decreased chemokine production, the ability of aged macrophages to traffic toward a chemoattractant is decreased with age [106-108]. Aged macrophages also exhibit impaired production of oxidative radicals and nitric oxide [103, 109, 110], reduced phagocytosis and clearance of pathogens [107, 111, 112], and decreased expression of MHC II and antigen presentation [113-117].

Although DCs are the major APC driving vaccine immune responses, macrophages are also APCs that contribute to this process. During vaccination with the Comirnaty or Spikevax SARS-(CoV-2 mRNA vaccines, macrophages exhibited NLRP3 inflammasome activation and underwent pyroptosis both of which served as innate immune triggers to drive adaptive immune responses [118]. Other studies have shown that vaccination increases M1 polarization post challenge, and that depletion of macrophages reduces the level of protection post vaccination and challenge [119, 120]. These studies suggest this M1 macrophages play a role in vaccine induced immune responses and are needed for protective immunity.

Since macrophages express ADA-1 receptors, A1R, A2aR, A2bR, A3R [122] and CD26 [123], presumably ADA-1 can influence the phentoype of macrophages as it does with DCs.

Accordingly, our hypothesis herein was two fold: (1) ADA-1 would modulate the cyotkine and chemokine profile of young macrophages and (2) ADA-1 would rescue age associated deficits in cytokine and chemokine production. To evaluate this, monocytes were isolated from PBMCs of young (<65 yrs) and aged (≥70 yrs) donors and differentiated into M0 or M1 macrophages with or without ADA-1 stimulation. Upon ADA-1 stimulation, TNFα and TL-6 expression was increased in both young and aged macrophages compared to unstimulated controls. Notably ADA-1 rescues age associated deficits in TNFα and TL-6 gene expression. We also observed that aged macrophages had increased expression of the chemokine CCL22 compared to young macrophages. These data demonstrate that ADA-1 enhances TL-6 and TNFα expression in young macrophages while also rescuing age associated deficits in TL-6 and TNFα expression. This study aids in elicidating the mechanism of ADA-1 as an adjuvant to enhance vaccine immune responses.

### Materials and Methods

### Human Samples

Blood samples were obtained from healthy donors at Martin Memorial Health Systems (FL). Consenting adults were screened using a questionnaire determining their demographic information, medication usage, and comorbidities. Participants were excluded with any acquired immunodefciency or immunomodulating medications (such as steroids, chemotherapy, or history of autoimmune disease), pregnancy, history of cancer and history of cirrhosis or renal failure, or antibiotic use within two weeks of recruitment. Blood samples were taken from individuals aged 18-80 and were made into single-cell PBMC suspensions and frozen in bovine serum albumin (Sigma) plus 10% DMSO (VWR) for cryopreservation in liquid nitrogen. The institutional review boards at the relevant institutions approved all procedures, and all participants provided signed informed consent.

### In Vitro Stimulation of Monocyte Derived Macrophages

Human monocytes were isolated using the StemCell negative selection kit (Catalog: 19059) per the manufacturer's guidance from cryogenically frozen PBMCs from healthy young (<65 yrs) and aged (≥70 yrs) donors. To polarize monocytes into M0 macrophages, monocytes were cultured with 20 ng/mL MCSF (Peprotech, Cat: 300-25) for 5 days. M0 macrophages were maintained by continued culturing with 20 ng/mL of recombinant human MCSF alone (M0 control) or were stimulated with 1 μM, 3 μM, 7 μM of recombinant ADA protein (Creative Enzymes, Cat: NATE-1009) for 48 hrs. M0 macrophages were polarized to M1 macrophages by culturing with 100 ng/mL of IFNγ and LPS (Sigma-Aldrich, Cat: L2654) alone (M1 control) for 48 hrs alone or were stimulated with 1p M, 3p M, 7 μM of recombinant ADA protein. M0 and M1 macrophages were harvested, RNA was isolated and used for PCR analysis for cytokine (IL-6, TNFα) and chemokine (CCL22) gene expression.

### PCR Analysis for Cytokine and Chemokine Gene Expression

RNA was extracted using the RNAqueous-Micro Total RNA Isolation Kit (Invitrogen, AM1931) and RNA quality and concentration was measured with a microplate reader. cDNA was synthesized using the High-Capacity cDNA Reverse Transcription Kit (Applied Biosystems, 4368814). qRT-PCR was conducted using custom oligonucleotides (ThermoFisher, Table 2) and Fast SYBR Green Master Mix (Applied Biosystems, 4385616). Statistical testing was performed using log-transformed data.

TABLE 2
PCR forward and reverse primer sequences

| | | SEQ ID |
|---|---|---|
| Gene | Sequence (5′ to 3′) | NO: |
| TNF forward | CCTCTCTCTAATCAGCCCTCTG | 12 |
| TNF reverse | GAGGACCTGGGAGTAGATGAG | 13 |
| IL6 forward | CCTGAACCTTCCAAAGATGGC | 14 |
| IL6 reverse | TTCACCAGGCAAGTCTCCTCA | 15 |
| CCL22 forward | GCGTGGTGTTGCTAACCTTCA | 16 |
| CCL22 reverse | AAGGCCACGGTCATCAGAGT | 17 |
| Housekeeping | CTGGAAGATGGACGTACTTTGT | 18 |
| RPS27A forward | C | |
| Housekeeping | CGACGAAGGCGACTAATTTTGC | 19 |
| RPS27A reverse | | |

### Statistics

All statistics were analyzed using GraphPad Prism 9 or 10. Error bars represent means±standard deviations. Mann-Whitney U test or Kruskal-Wallis ANOVA statistical tests were performed where denotated to determine statistical differences between groups. In all data, *p<0.05, **p 0.01, ***p 0.001, and ****p<0.0001.

### Results

### ADA-1 Rescues Macrophage Age Associated Deficits in TNFα Expression

To evaluate effect of ADA-1 on the cyotkine and chemokine profile of young macrophages and aged macrophages, monocytes were isolated from PBMCs of young (<65 yrs) and aged (≥70 yrs) donors (see Table 3 for patient demographics) and differentiated into M0 macrophages (M0 control) or polarized into M1 macrophages (M1 control). A subset of M0 and M1 macrophages were stimulated with 1 μM, 3 μM or 7 μM of ADA-1. ADA-1 stimulated and unstimulated M0 and M1 macrophages were harvested for PCR analysis to evaluate for cytokine (IL-6, TNFα) and chemokine (CCL22) gene expression. The experimental design is depicted in FIG. 16.

TABLE 3
Human Patient Demographics

| Patient ID | Young or Old | Sex | Age (years |
|---|---|---|---|
| HAD-HLTHY-1 | Young | Male | 40 |
| HAD-HLTHY-6 | Young | N/A | <65 |
| HAD-HLTHY-7 | Young | N/A | <65 |
| DH006 | Young | Male | 53 |
| LH074 | Old | Male | 79 |
| LH098 | Old | Female | 75 |
| LH0101 | Old | Female | 70 |
| LH062 | Old | Female | 81 |

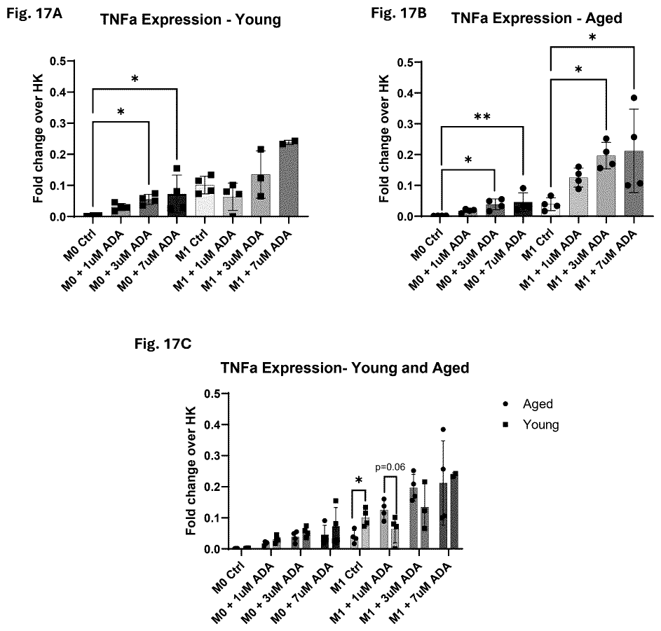

When evaluating TNFα gene expression in young macrophages, we observed that M0 macrophages had significantly increased TNFα gene expression when stimulated with 3 μM (mean 0.0551, *p=0.0203) and 7 μM (mean 0.0729, *p=0.0472) of ADA-1 compared to unstimulated M0 macrophages (mean 0.0024). In young M1 macrophages, we observed a trend of increased TNFα gene expression when stimulated with 7 μM of ADA-1 (mean 0.2382) however, not significant when compared to unstimulated M1 macrophages (mean 0.1012) (FIG. 17A). Similar to young M0 macrophages, aged M0 macrophages exhibited significantly increased TNFα gene expression when stimulated with 3 μM (mean 0.0385, *p=0.0180) and 7 μM (mean 0.456, **p=0.0089) of ADA-1 compared to unstimulated M0 macrophages (mean 0.0016).

In contrast to young M1 macrophages, aged M1 macrophages exhibited significantly increased TNFα gene expression when stimulated with 3 μM (mean 0.1965, *p=0.0113) and 7 μM (mean 0.2120, *p=0.0347) of ADA-1 compared to unstimulated M1 macrophages (mean 0.0390) (FIG. 17B). When comparing TNFα gene expression in young and old M0 macrophages, we observe no difference in TNFα gene expression independent of age and ADA-1 stimulation. However, when comparing young and old M1 macrophages without ADA-1 stimulation, we observe that aged M1 macrophages exhibited a significant decrease (mean 0.0390, *p=0.0286) in TNFα gene expression compared to young M1 macrophages (mean 0.1012) indicating there is an aged associated deficit in TNFα gene expression in M1 macrophages. Upon ADA-1 stimulation, we observe no difference between young and aged M1 macrophages TNFα gene expression. This was the case for the 1 μM (mean young 0.0635, mean old 0.1254) 3 μM (mean young 0.1349, mean old 0.1965) and 7 μM (mean young 0.2382, mean old 0.2120) ADA-1 stimulation (FIG. 17C).

These data demonstrate that ADA-1 enhances TNFα gene expression in both young and aged M0 macrophages particularly at the 3 μM and 7 μM doses (FIG. 17A-17B). ADA-1 stimulation also enhanced TNFα gene expression in aged M1 macrophages particularly at the 3 μM and 7 μM doses (FIG. 17B). Additionally, these data demonstrate that ADA-1 stimulation at doses as low as 1 μM rescues the age associated deficit in TNFα gene expression in aged M1 macrophages (FIG. 17C).

### ADA-1 Increases IL-6 Expression in Young Macrophages and Aged Macrophages

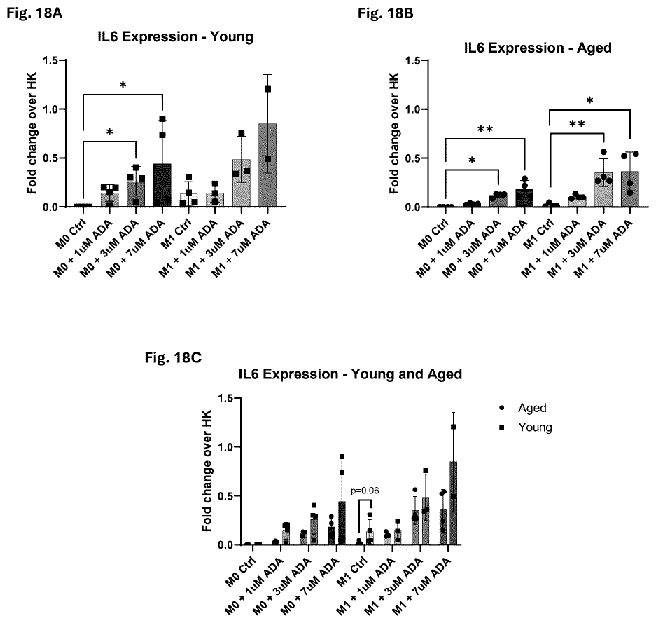

IL-6 expression was evaluated in young and aged M0 and M1 macrophages that remained unstimulated or were stimulated with ADA-1 as outlined in FIG. 16. We observed that young M0 macrophages had significantly increased IL-6 gene expression when stimulated with 3 μM (mean 0.2612, *p=0.0280) and 7 μM (mean 0.4414, *p=0.0225) of ADA-1 compared to unstimulated M0 macrophages (mean 0.00001). In young M1 macrophages, we observed a trend of increased IL-6 gene expression when stimulated with 3 μM of ADA-1 (mean 0.4861) 7 μM of ADA-1 (mean 0.8495) however, the increase was not significant when compared to unstimulated M1 macrophages (mean 0.1342) (FIG. 18A). Similar to young M0 macrophages, aged M0 macrophages exhibited significantly increased IL-6 gene expression when stimulated with 3 μM (mean 0.1192, *p=0.0143) and 7 μM (mean 0.1830, **p=0.0054) of ADA-1 compared to unstimulated M0 macrophages (mean 0.00004). In contrast to young M1 macrophages, aged M1 macrophages exhibited significantly increased IL-6 gene expression when stimulated with 3p M (mean 0.3515, **p=0.0054) and 7 μM (mean 0.3640, *p=0.0143) of ADA-1 compared to unstimulated M1 macrophages (mean 0.01830) (FIG. 18B). When comparing IL-6 gene expression in young and old M0 macrophages, we observe no difference in IL-6 gene expression independent of age and ADA-1 stimulation. However, when comparing young and old M1 macrophages without ADA-1 stimulation, we observe that aged M1 macrophages exhibited a near significant decrease (mean 0.01830, p=0.06) in IL-6 gene expression compared to young M1 macrophages (mean 0.03768) suggesting a modest aged associated deficit in IL-6 gene expression in M1 macrophages. Upon ADA-1 stimulation, we observe no difference between young and aged M1 macrophages IL-6 gene expression. This was the case for the 1 μM (mean young 0.05042, mean old 0.1037) 3 μM (mean young 0.3368, mean old 0.3515) and 7 μM (mean young 0.4931, mean old 0.3640) ADA-1 stimulation (FIG. 18C).

These data demonstrate that ADA-1 enhances IL-6 gene expression in both young and aged M0 macrophages particularly at the 3 μM and 7 μM doses (FIG. 18A-18B). ADA-1 stimulation also enhanced IL-6 gene expression in aged M1 macrophages particularly at the 3 μM and 7 μM doses (FIG. 18B). Additionally, these data demonstrate that ADA-1 stimulation at doses as low as 1 μM rescues a modest age associated deficit observed in IL-6 gene expression in aged M1 macrophages (FIG. 18C).

### CCL22 Gene Expression is Enhanced in Aged Macrophages Independent of ADA-1

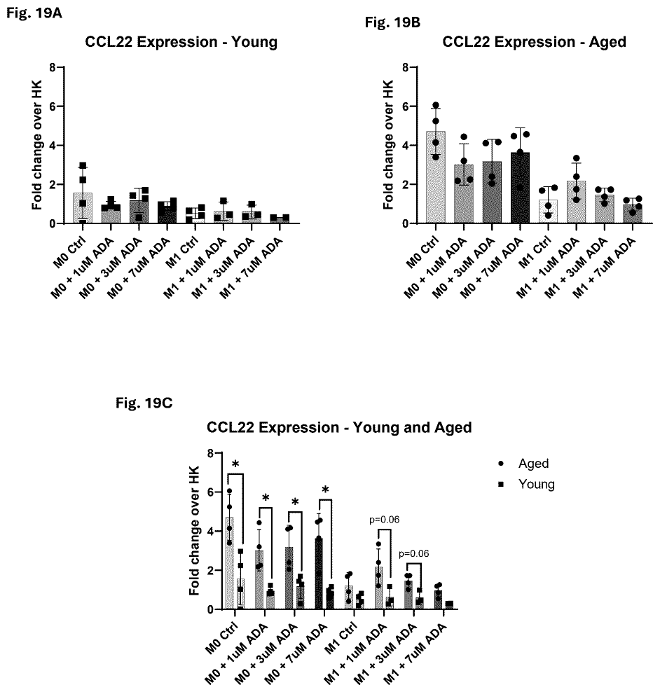

CCL22 expression was evaluated in young and aged M0 and M1 macrophages that remained unstimulated or were stimulated with ADA-1 as outlined in FIG. 16. In young M0 and M1 macrophages we observe no difference in CCL22 gene expression independent of ADA-1 stimulation (FIG. 19A). This was also true for aged M0 and M1 macrophages where CCL22 gene expression was not impacted by ADA-1 stimulation (FIG. 19B). However, when comparing young and aged M0 macrophages, we observed a significant increase in CCL22 gene expression in aged M0 macrophages (mean 4.708, *p=0.0286) compared to young M0 macrophages (mean 1.558) (FIG. 19C). CCL22 gene expression in aged M0 macrophages remained elevated compared to young M0 macrophages during stimulation with 1 μM (mean young 0.9380, mean old 3.017, *p=0.0286) 3 μM (mean young 1.175, mean old 3.180, *p=0.0286) and 7 μM (mean young 0.8915, mean old 3.632, *p=0.0286) of ADA-1 indicating that ADA-1 does not influence the age associated increase in CCL22 gene expression in M0 macrophages. Comparing young and aged M1 macrophages without ADA-1 stimulation, there was no difference in CCL22 gene expression observed (mean young 0.5157, mean old 1.204). ADA-1 stimulation at the 1 μM (mean young 0.6317, mean old 2.171) and 3 μM (mean young 0.6010, mean old 1.458) doses resulted in a near significant increase (p=0.06) in CCL22 expression in aged M1 macrophages compared to young M1 macrophages (FIG. 19C).

These data demonstrate an age associated increase in CCL22 gene expression in M0 macrophages that is not influenced by ADA-1. In aged M1 macrophages ADA-1 stimulation slightly exacerbated this age associated increase in CCL22 gene expression.

### Discussion

Since macrophages express ADA-1 receptors [122][123] and ADA-1 has been shown to effect DC maturation and cytokine expression [25], we hypothesized ADA-1 would influence the phentoype macrophages. Additionally, since macrophages contribute to vaccine induced immune responses and ADA-1 can resuce age associated deficits in vaccine responses, we hypothesized that macrophage phenotypic changes driven by ADA-1 would provide a mechanistic understanding as to how ADA-1 enhances vaccine responses in both young [31] and aged mice [32].

Herein, we demonstrate that ADA-1 enhances TNFα and IL-6 gene expression in macrophages from both young (<65 yrs and aged donors (>70 yrs). Notably ADA-1 rescues an age associated deficit in TNFα and IL-6 gene expression (see FIG. 17-18). The age associated deficit we observed here in TNFα [100, 102-104] and IL-6 [100, 103] expression is in line with other previously reported studies.

TNFα is known to drive DC activation and maturation [125] which is complementary to our previous findings of ADA-1 itself also drives DC maturation [25]. The ADA-1 induced production of TNFα in macrophages could therefore be working synergistically to ADA-1 induced DC maturation. TNFα is also known to induce recruited monocytes to differentiate into DCs [126]. TNFα has also been shown to be required for the initial priming phase of antigen specific immunity [127]. ADA-1 induced TNFα expression from macrophages could therefore be contributing to the enhanced antigen specific immunity we observe upon ADA-1 co-immunization [25, 32](Example 1-2).

The ADA-1 driven increase in IL-6 gene expression in macrophages likely works synergistically with IL-6 production that is upregulated in ADA-1 matured DCs [25]. IL-6 is known to increase B cell antibody production, increase differentiation of cytotoxic CD8⁺ T cells, and promote TFH differentiation [128]. ADA-1 induced IL-6 expression from macrophages as well as DCs thereby contributing to the increased antibody, increased CD8⁺ T cell functionality (FIGS. 5, 8, and 9) and increased antigen specific TFH. IL-6 is also known to inhibit regulatory T cell (Treg) differentiation [129]. Increased frequency of Tregs have been reported in the secondary lymphoid organs of aged mice [130] and the ratio of Tregs to effector T cells has also been reported to be increased with age [131]. Tregs also contribute to the suboptimal vaccine responses by limiting antibody production [130]. ADA-1 induced IL-6 expression from macrophages and DCs is presumably preventing Treg differentiation leading to decreased frequencies of Tregs and therefore restored vaccine responses we observe in aged mice [124].

Additionally, we found that aged macrophages upregulate CCL22 expression compared to young macrophages (see FIG. 19). CCL22 has been found to be involved in Treg recruitment [92] and likely contributes to the Treg mediated damping of vaccine responses in aged individuals [130]. Since CCL22 expression in aged macrophages was independent of ADA-1 stimulation, presumably ADA-1 does not impact Treg recruitment via CCL22 but rather inhibits Treg differentiation and subsequently decreases overall frequencies of Tregs through an IL-6 dependent mechanism.

These data demonstrate that ADA-1 enhances IL-6 and TNFα expression in young macrophages while also rescuing age associated deficits in IL-6 and TNFα expression. These data provide a deeper understanding of the mechanism of ADA-1 as a vaccine adjuvant. Adjuvant mechanism characterization is becoming increasingly desirable from a regulatory and licensing perspective as it accelerates and derisks clinical translation [20]. These data therefore contribute to the mechanistic understand of ADA-1 and could facilitate its clinical translation.

### Example III: Summary of Results, Discussion, and Conclusions

While SARS-CoV-2 vaccines have demonstrated immunogenicity and protection against severe disease, several studies have demonstrated waning humoral responses, decreased efficacy particularly against VOCs, and a lack of durable immune responses that protect against mild symptomatic disease. To this end, there is a continued need for next generation vaccines, alternative vaccine platforms, and vaccine adjuvants to induced durable, protective anti-SARS-CoV-2 immunity.

In terms of HIV vaccines, no immunogen or adjuvant has been able to induce bNAbs against HIV needed for protective immunity. There is a continued need for a prophylactic vaccine that will provide protective immunity against HIV and prevent the continued spread of HIV. Additionally, there are limited vaccine adjuvants that are approved for human use with limited knowledge of their mechanism of action.

### ADA Enhances HIV Specific Humoral and Cellular Immunity

ART has transformed HIV from a once fatal disease to a now chronic yet manageable disease [8]. This is an undisputed success in the field. However, even with ART, HIV continues to spread to an estimated 1.5 million people per year. A prophylactic vaccine to prevent HIV acquisition remains a major unmet need in the field despite many years of failed attempts [9, 12]. The high sequence diversity and multiple routes of transmission of HIV necessitates an effective HIV vaccine to induce both circulating and mucosal bNAbs.

Developing an effective HIV vaccine immunogen has posed challenges since the HIV Env protein is extremely metastable and heavily glycosylated, resulting in very little access to immune recognition of neutralizing epitopes and subsequent immune evasion [10, 11]. Identifying HIV vaccine adjuvants that promote reliable and robust SHM has also proved challenging [35, 36].

Since ADA-1 is critical to TFH programing, induces TFH differentiation, prolongs TFH survival [27] and TFH are necessary for germinal centers and are critical drivers SHM [30] ADA-1 could help drive SHM needed for an effective HIV vaccine. To begin to address gaps in the field of improved HIV vaccine immunogens and adjuvants, we evaluated a DNA encoded NTL antigen pBG505-TTT with the novel molecular adjuvant ADA-1 in mice (Example 1).

We found that ADA-1 enhanced HIV specific IgG magnitude (FIG. 1) and isotype switching at doses as low as 1 μg pBG505-TTT. In terms of antibody isotypes, ADA-1 increased the magnitude of HIV specific IgG1, IgG2a and IgG2b (FIG. 4). HIV specific antibody magnitude and isotype switching was not further enhanced by ADA-1 at 5 μg pBG505-TTT clearly demonstrating that ADA-1 has a dose sparing effect. Interestingly, we found that ADA-1 uniquely drives HIV specific IgG2a responses that was not observed in pBG505-TTT immunization alone (FIG. 4). Additionally, we demonstrate that ADA-1 increases the functionality of HIV specific antibody as evidenced by increased ADCD (FIG. 1) and neutralization (FIG. 14). Characterizing cellular responses, we found ADA-1 enhances HIV specific T cell cytokine production (FIG. 5), polyfunctionality, degranulation (FIG. 8) and activation (FIG. 12). Lastly, we demonstrate that ADA-1 enhances the frequency of HIV specific memory B cells (FIG. 12).

Taken together, this study demonstrates that ADA-1 enhances HIV specific humoral and cellular immune responses notably at very low doses of pBG505-TTT. This study focused on characterizing immunogenicity and showed that HIV specific IgG remained elevated across timepoints (D41P1, D21P2, D14P3 immunizations) in ADA-1 immunized mice (FIG. 1).

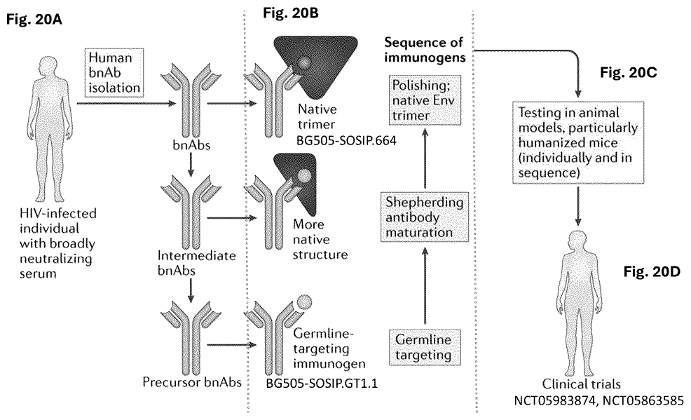

We begin to address ADA-1 driven antibody neutralization here by demonstrating that ADA-1 enhances neutralization of a tier 1A pseudovirus HIV Q461eTAIV (FIG. 3.14). Recent HIV vaccine strategies are using sequential approach starting with a germ line targeting priming vaccination, followed by heterologous boosting to induce memory B cells producing bNABs (see FIG. 20). Given the role of ADA-1 in boosting GC responses [27], ADA-1 could be incorporated into this strategy.

### ADA Drives IL-6 and TNFα Expression in Macrophages

Macrophages function as an important bridge between innate and adaptive immunity by presenting antigen for the priming and activation of adaptive immune cells [88, 93]. During aging there is a progressive decline in the function of the immune system known as immunosenescence [88] which contributes to decreased immune responses in aged populations to several vaccines, such as influenza [94-96], hepatitis B virus [97, 98], and SARS-CoV-2 [99]. Several studies have demonstrated that aging negatively impacts macrophage function [100, 101, 103]. We therefore sought to understand if ADA-1 impacts human macrophage cytokine and chemokine production in vitro to provide a mechanistic understanding for ADA-1 enhanced vaccine responses observed in vivo in both young and aged mice [124].

We found that ADA-1 increased TNFα and IL-6 expression in both young and aged macrophages. Notably ADA-1 rescued an age associated deficit in TNFα and IL-6 gene expression (FIG. 17-18). Independent of ADA-1 stimulation we observed that aged macrophages had increased expression of the chemokine CCL22 compared to young macrophages (FIG. 19).

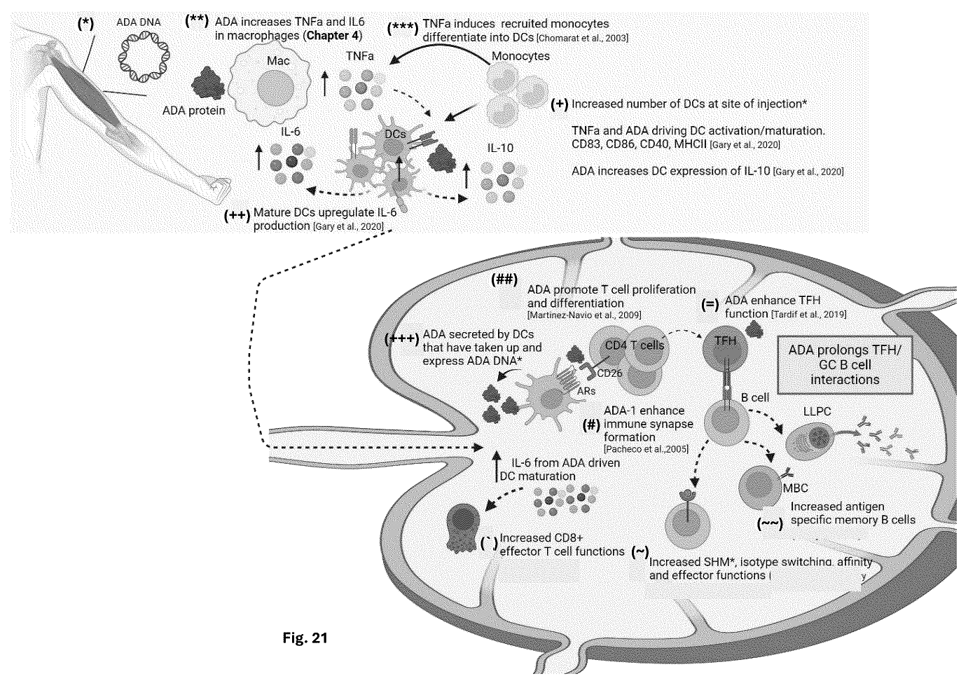

A mechanism of ADA-1 based on these findings is summarized in FIG. 21. This mechanism indicates that ADA-1 driven TNFα production in macrophages is supporting DC activation and maturation [125] and differentiation of recruited monocytes into DCs resulting in an increased frequency of DCs at the site on injection [126]. ADA-1 driven IL-6 production from macrophages is supporting increased B cell antibody production, increased differentiation of cytotoxic CD8⁺ T cells, and promoting TFH differentiation [128]. Additionally, ADA-1 driven IL-6 production could be inhibiting Treg differentiation [129] therefore decreasing overall frequencies of Tregs and correcting for Treg mediated suboptimal vaccine responses [130] in aged mouse models [32].

### Mechanism of ADA-1 as a Vaccine Adjuvant

Based on the findings outlined herein, a mechanism of ADA-1 as a molecular vaccine adjuvant is summarized in FIG. 21. Upon injection and electroporation, ADA-1 encoded DNA is taken up by cells at the injection site resulting in ADA-1 protein expression (FIG. 21 at *). ADA-1 protein can then bind to adenosine receptors on macrophages [122] to drive TNFα and IL-6 production (FIG. 21 at **).

ADA-1 driven TNFα production from macrophages can then induce recruited monocytes to differentiate into DCs [126] resulting in increased frequencies of DCs at the site of injection (FIG. 21 at *** and +). In addition to macrophages, ADA-1 protein can bind to adenosine receptors on DCs [28] resulting in increased activation and maturation of DCs as evidenced by increased CD83, CD86, CD40 and MHCII expression [25](FIG. 21 at +). ADA-1 can also drive production of IL-6 from ADA-1 matured DCs and increases DC production of IL-10 [25] (FIG. 21 at + and ++).

DCs that have taken up ADA-1 encoded DNA at the site of injection can migrate to secondary lymphoid organs, express and secrete ADA-1 protein (FIG. 21 at +++). ADA-1 protein can then bind to adenosine receptors on DCs within secondary lymphoid organs and CD26 on T cells to enhance immune synapse formation [28](FIG. 21 at #). ADA-1 binding to receptors on T cell can also drive enhanced T cell proliferations and differentiation [39](FIG. 21 at ##). CD4⁺ T cell that have differentiated into TFH cells can also bind to ADA-1 to enhance the function of TFH resulting in improved B cell help and prolongs the interaction of TFH and GC B cells [27] (FIG. 21 at =).

This prolonged TFH:GC B cell interaction facilitates the development of enhanced adaptive immune responses we see with ADA-1 co-immunization such as increased frequencies of antigen specific memory B cells (FIG. 21 at ˜˜) as well as increase antigen specific antibody magnitude, isotype switching, affinity, functionality (FIG. 21 at ˜) [25] [32] and increased SHM. Lastly, ADA-1 matured DCs producing increased IL-6 that have migrated to secondary lymphoid organs can drive increased CD8⁺ T cell effector functions (FIG. 21 at ′).

Next generation SARS-CoV-2 vaccines are needed to induce durable, protective immunity against SARS-CoV-2 to discontinue the spread of new infections. Although the COVID-19 pandemic has halted, the HIV pandemic continues despite the availability of ART. To this end, the HIV field desperately needs a prophylactic vaccine to induce protective immunity against HIV. Vaccine adjuvant development, until recently, has been slow to progress and yet, there are still very limited adjuvants available for use in humans with limited known about their mechanism of action. Difficult to target pathogens may require novel, more potent adjuvants to drive the specific immunity needed for protection. Additionally, populations known to mount suboptimal vaccine responses such as aged and infant populations may require novel adjuvants to develop a sufficient vaccine response. The work presented here advances the field's understanding of precision adjuvants, such as ADA-1, that specifically target key orchestrators (TFH cells, APCs) of robust, durable, and protective vaccine induced immunity. This work also provides a step forward for DNA vaccination and demonstrates the effectiveness of this platform against SARS-CoV-2 and HIV antigens, providing a basis for DNA vaccination to be expanded to other pathogenic antigens.

### LIST OF REFERENCES

1. Estimating global, regional, and national daily and cumulative infections with SARS-CoV-2 through Nov. 14, 2021: a statistical analysis. Lancet, 2022. 399(10344): p. 2351-2380.
2. Zasada, A. A., et al., COVID-19 Vaccines over Three Years after the Outbreak of the COVID-19 Epidemic. Viruses, 2023. 15(9): p. 1786.
3. Wu, K., et al., Preliminary Analysis of Safety and Immunogenicity of a SARS-CoV-2 Variant Vaccine Booster. medRxiv, 2021: p. 2021.05.05.21256716.
4. Naaber, P., et al., Declined antibody responses to COVID-19 mRNA vaccine within first three months. medRxiv, 2021: p. 2021.04.19.21255714.
5. Hertz, T., et al., Correlates of protection for booster doses of the SARS-CoV-2 vaccine BNT162b2. Nature Communications, 2023. 14(1): p. 4575.
6. McMahan, K., et al., Correlates of protection against SARS-CoV-2 in rhesus macaques. Nature, 2021. 590(7847): p. 630-634.
7. Benkeser, D., et al., Immune correlates analysis of a phase 3 trial of the AZD1222 (ChAdOx1 nCoV-19) vaccine. npj Vaccines, 2023. 8(1): p. 36.
8. Barré-Sinoussi, F., A. L. Ross, and J.-F. Delfraissy, Past, present and future: 30 years of HIV research. Nature Reviews Microbiology, 2013. 11(12): p. 877-883.
9. Landovitz, R. J., H. Scott, and S. G. Deeks, Prevention, treatment and cure of HIV infection. Nature Reviews Microbiology, 2023. 21(10): p. 657-670.
10. Kim, J., et al., Current approaches to HIV vaccine development: a narrative review. Journal of the International AIDS Society, 2021. 24(S7): p. e25793.
11. Sanders, R. W., et al., A next-generation cleaved, soluble HIV-1 Env trimer, BG505 SOSIP.664 gp140, expresses multiple epitopes for broadly neutralizing but not non-neutralizing antibodies. PLoS Pathog, 2013. 9(9): p. e1003618.
12. Ng'uni, T., C. Chasara, and Z. M. Ndhlovu, Major Scientific Hurdles in HIV Vaccine Development: Historical Perspective and Future Directions. Frontiers in Immunology, 2020. 11.
13. Flynn, N. M., et al., Placebo-controlled phase 3 trial of a recombinant glycoprotein 120 vaccine to prevent HIV-1 infection. J Infect Dis, 2005. 191(5): p. 654-65.
14. Gilbert, P. B., et al., Correlation between immunologic responses to a recombinant glycoprotein 120 vaccine and incidence of HIV-1 infection in a phase 3 HIV-1 preventive vaccine trial. J Infect Dis, 2005. 191(5): p. 666-77.
15. Pitisuttithum, P., et al., Randomized, double-blind, placebo-controlled efficacy trial of a bivalent recombinant glycoprotein 120 HIV-1 vaccine among injection drug users in Bangkok, Thailand. J Infect Dis, 2006. 194(12): p. 1661-71.
16. Sanders, R. W., et al., Stabilization of the Soluble, Cleaved, Trimeric Form of the Envelope Glycoprotein Complex of Human Immunodeficiency Virus Type 1. Journal of Virology, 2002. 76(17): p. 8875-8889.
17. Binley, J. M., et al., A Recombinant Human Immunodeficiency Virus Type 1 Envelope Glycoprotein Complex Stabilized by an Intermolecular Disulfide Bond between the gp120 and gp41 Subunits Is an Antigenic Mimic of the Trimeric Virion-Associated Structure. Journal of Virology, 2000. 74(2): p. 627-643.
18. de Taeye, S. W., et al., Immunogenicity of Stabilized HIV-1 Envelope Trimers with Reduced Exposure of Non-neutralizing Epitopes. Cell, 2015. 163(7): p. 1702-15.
19. McCoy, L. E., et al., Holes in the Glycan Shield of the Native HIV Envelope Are a Target of Trimer-Elicited Neutralizing Antibodies. Cell Rep, 2016. 16(9): p. 2327-38.
20. Nanishi, E., D. J. Dowling, and O. Levy, Toward precision adjuvants: optimizing science and safety. Curr Opin Pediatr, 2020. 32(1): p. 125-138.
21. Pifferi, C., R. Fuentes, and A. Fernández-Tejada, Natural and synthetic carbohydrate-based vaccine adjuvants and their mechanisms of action. Nature Reviews Chemistry, 2021.
22. Pulendran, B., P. S. Arunachalam, and D. T. O'Hagan, Emerging concepts in the science of vaccine adjuvants. Nature Reviews Drug Discovery, 2021. 20(6): p. 454-475.
23. Buckley, R. H., Molecular Defects in Human Severe Combined Immunodeficiency and Approaches to Immune Reconstitution. Annual Review of Immunology, 2004. 22(1): p. 625-655.
24. Qu, Y., et al., Adenosine Deaminase 1 Overexpression Enhances the Antitumor Efficacy of Chimeric Antigen Receptor-Engineered T Cells. Human Gene Therapy, 2021. 33(5-6): p. 223-236.
25. Gary, E., et al., Adenosine deaminase-1 enhances germinal center formation and functional antibody responses to HIV-1 Envelope DNA and protein vaccines. Vaccine, 2020. 38(22): p. 3821-3831.
26. Flinn, A. M. and A. R. Gennery, Adenosine deaminase deficiency: a review. Orphanet Journal of Rare Diseases, 2018. 13(1): p. 65.
27. Tardif, V., et al., Adenosine deaminase-1 delineates human follicular helper T cell function and is altered with HIV. Nature Communications, 2019. 10(1): p. 823.
28. Pacheco, R., et al., CD26, adenosine deaminase, and adenosine receptors mediate costimulatory signals in the immunological synapse. Proceedings of the National Academy of Sciences of the United States of America, 2005. 102(27): p. 9583-9588.
29. Dorsey, M. J., et al., PEGylated Recombinant Adenosine Deaminase Maintains Detoxification and Lymphocyte Counts in Patients with ADA-SCID. J Clin Immunol, 2023. 43(5): p. 951-964.
30. Locci, M., et al., Human circulating PD-1+CXCR3-CXCR5+ memory Tfh cells are highly functional and correlate with broadly neutralizing HIV antibody responses. Immunity, 2013. 39(4): p. 758-69.
31. Cusimano, G. M., et al., Improved Durability to SARS-CoV-2 Vaccine Immunity following Coimmunization with Molecular Adjuvant Adenosine Deaminase-1. The Journal of Immunology, 2022: p. ji2200056.
32. Gary, E. N., et al., Adenosine deaminase augments SARS-CoV-2 specific cellular and humoral responses in aged mouse models of immunization and challenge. Frontiers in Immunology, 2023. 14.
33. Kale, A., et al., An Overview of Current Accomplishments and Gaps of COVID-19 Vaccine Platforms and Considerations for Next Generation Vaccines. Journal of Pharmaceutical Sciences, 2023. 112(5): p. 1345-1350.
34. Zhang, G., et al., mRNA vaccines in disease prevention and treatment. Signal Transduction and Targeted Therapy, 2023. 8(1): p. 365.
35. Ratnapriya, S., et al., Adjuvant-mediated enhancement of the immune response to HIV vaccines. The FEBS Journal, 2022. 289(12): p. 3317-3334.
36. Francica, J. R., et al., Analysis of immunoglobulin transcripts and hypermutation following SHIVAD8 infection and protein-plus-adjuvant immunization. Nature Communications, 2015. 6(1): p. 6565.
37. Leroux-Roels, G., Unmet needs in modern vaccinology: Adjuvants to improve the immune response. Vaccine, 2010. 28: p. C25-C36.
38. Martinez-Navio, J. M., et al., Immunological dysfunction in HIV-1-infected individuals caused by impairment of adenosine deaminase-induced costimulation of T-cell activation. Immunology, 2009. 128(3): p. 393-404.
39. Martinez-Navio, J. M., et al., Adenosine deaminase potentiates the generation of effector, memory, and regulatory CD4+ T cells. J Leukoc Biol, 2011. 89(1): p. 127-36.
40. Smith, T. R. F., et al., Immunogenicity of a DNA vaccine candidate for COVID-19. Nature Communications, 2020. 11(1): p. 2601.
41. Reiss, S., et al., Comparative analysis of activation induced marker (AIM) assays for sensitive identification of antigen-specific CD4 T cells. PloS one, 2017. 12(10): p. e0186998.
42. Hu, J. K., et al., Murine Antibody Responses to Cleaved Soluble HIV-1 Envelope Trimers Are Highly Restricted in Specificity. Journal of Virology, 2015. 89(20): p. 10383-10398.
43. Sanders, R. W., et al., HIV-1 neutralizing antibodies induced by native-like envelope trimers. Science, 2015. 349(6244): p. aac4223.
44. del Moral-Sánchez, I., et al., Triple tandem trimer immunogens for HIV-1 and influenza nucleic acid-based vaccines. 2023.
45. Xu, Z., et al., Induction of tier-2 neutralizing antibodies in mice with a DNA-encoded HIV envelope native like trimer. Nature Communications, 2022. 13(1): p. 695.
46. Havenar-Daughton, C., et al., Direct Probing of Germinal Center Responses Reveals Immunological Features and Bottlenecks for Neutralizing Antibody Responses to HIV Env Trimer. Cell Rep, 2016. 17(9): p. 2195-2209.
47. Schoofs, T., et al., HIV-1 therapy with monoclonal antibody 3BNC117 elicits host immune responses against HIV-1. Science, 2016. 352(6288): p. 997-1001.
48. Pegu, A., et al., Neutralizing antibodies to HIV-1 envelope protect more effectively in vivo than those to the CD4 receptor. Sci Transl Med, 2014. 6(243): p. 243ra88.
49. Su, B., et al., Update on Fc-Mediated Antibody Functions Against HIV-1 Beyond Neutralization. Front Immunol, 2019. 10: p. 2968.
50. Parsons, M. S., A. W. Chung, and S. J. Kent, Importance of Fc-mediated functions of anti-HIV-1 broadly neutralizing antibodies. Retrovirology, 2018. 15(1): p. 58.
51. Bournazos, S. and J. V. Ravetch, Anti-retroviral antibody FcγR-mediated effector functions. Immunol Rev, 2017. 275(1): p. 285-295.
52. Mayr, L. M., B. Su, and C. Moog, Non-Neutralizing Antibodies Directed against HIV and Their Functions. Front Immunol, 2017. 8: p. 1590.
53. Lewis, G. K., et al., Beyond Viral Neutralization. AIDS Res Hum Retroviruses, 2017. 33(8): p. 760-764.
54. Holl, V., et al., Nonneutralizing antibodies are able to inhibit human immunodeficiency virus type 1 replication in macrophages and immature dendritic cells. J Virol, 2006. 80(12): p. 6177-81.
55. Excler, J. L., et al., Nonneutralizing functional antibodies: a new “old” paradigm for HIV vaccines. Clin Vaccine Immunol, 2014. 21(8): p. 1023-36.
56. Laidlaw, B. J., et al., Cooperativity between CD8+ T cells, non-neutralizing antibodies, and alveolar macrophages is important for heterosubtypic influenza virus immunity. PLoS Pathog, 2013. 9(3): p. e1003207.
57. Zander, R., et al., Tfh-cell-derived interleukin 21 sustains effector CD8+ T cell responses during chronic viral infection. Immunity, 2022. 55(3): p. 475-493.e5.
58. Monel, B., et al., HIV Controllers Exhibit Effective CD8(+) T Cell Recognition of HIV-1-Infected Non-activated CD4(+) T Cells. Cell Rep, 2019. 27(1): p. 142-153.e4.
59. Picker, L. J., et al., Programming cytomegalovirus as an HIV vaccine. Trends Immunol, 2023. 44(4): p. 287-304.
60. Torrents de la Peña, A., et al., Improving the Immunogenicity of Native-like HIV-1 Envelope Trimers by Hyperstabilization. Cell Rep, 2017. 20(8): p. 1805-1817.
61. Steichen, J. M., et al., HIV Vaccine Design to Target Germline Precursors of Glycan-Dependent Broadly Neutralizing Antibodies. Immunity, 2016. 45(3): p. 483-496.
62. del Moral-Sánchez, I., et al., High thermostability improves neutralizing antibody responses induced by native-like HIV-1 envelope trimers. npj Vaccines, 2022. 7(1): p. 27.
63. Malherbe, D. C., et al., Sequential Immunization with a Subtype B HIV-1 Envelope Quasispecies Partially Mimics the In Vivo Development of Neutralizing Antibodies. Journal of Virology, 2011. 85(11): p. 5262-5274.
64. Fischinger, S., et al., A high-throughput, bead-based, antigen-specific assay to assess the ability of antibodies to induce complement activation. Journal of Immunological Methods, 2019. 473: p. 112630.
65. Aktas, E., et al., Relationship between CD107a expression and cytotoxic activity. Cell Immunol, 2009. 254(2): p. 149-54.
66. Bettini, E. and M. Locci, SARS-CoV-2 mRNA Vaccines: Immunological Mechanism and Beyond. Vaccines, 2021. 9(2).
67. Huber, V. C., et al., Distinct Contributions of Vaccine-Induced Immunoglobulin G1 (IgG1) and IgG2a Antibodies to Protective Immunity against Influenza. Clinical and Vaccine Immunology, 2006. 13(9): p. 981-990.
68. Hovden, A. O., R. Cox, and L. Haaheim, Whole influenza virus vaccine is more immunogenic than split influenza virus vaccine and induces primarily an IgG2a response in BALB/c mice. Scandinavian journal of immunology, 2005. 62(1): p. 36-44.
69. Huber, V. C., et al., Fc receptor-mediated phagocytosis makes a significant contribution to clearance of influenza virus infections. The Journal of Immunology, 2001. 166(12): p. 7381-7388.
70. Moran, T. M., et al., Th2 responses to inactivated influenza virus can be converted to Th1 responses and facilitate recovery from heterosubtypic virus infection. The Journal of infectious diseases, 1999. 180(3): p. 579-585.
71. Arulanandam, B. P., M. O'Toole, and D. W. Metzger, Intranasal interleukin-12 is a powerful adjuvant for protective mucosal immunity. The Journal of infectious diseases, 1999. 180(4): p. 940-949.
72. Bruhns, P., Properties of mouse and human IgG receptors and their contribution to disease models. Blood, 2012. 119(24): p. 5640-5649.
73. Nimmerjahn, F., et al., FcgammaRIV: a novel FcR with distinct IgG subclass specificity. Immunity, 2005. 23(1): p. 41-51.
74. HeB, R., et al., Glycosylation of HIV Env Impacts IgG Subtype Responses to Vaccination. Viruses, 2019. 11(2).
75. Ackerman, M. E., et al., Polyfunctional HIV-Specific Antibody Responses Are Associated with Spontaneous HIV Control. PLOS Pathogens, 2016. 12(1): p. e1005315.
76. Collins, D. R., G. D. Gaiha, and B. D. Walker, CD8+ T cells in HIV control, cure and prevention. Nature Reviews Immunology, 2020. 20(8): p. 471-482.
77. Yang, O. O., et al., Efficient lysis of human immunodeficiency virus type 1-infected cells by cytotoxic T lymphocytes. Journal of virology, 1996. 70(9): p. 5799-5806.
78. Sacha, J. B., et al., Gag-specific CD8+ T lymphocytes recognize infected cells before AIDS-virus integration and viral protein expression. The Journal of Immunology, 2007. 178(5): p. 2746-2754.
79. Heydarchi, B., et al., Broad and ultra-potent cross-clade neutralization of HIV-1 by a vaccine-induced CD4 binding site bovine antibody. Cell Rep Med, 2022. 3(5): p. 100635.
80. Pauthner, M., et al., Elicitation of Robust Tier 2 Neutralizing Antibody Responses in Nonhuman Primates by HIV Envelope Trimer Immunization Using Optimized Approaches. Immunity, 2017. 46(6): p. 1073-1088.e6.
81. Verkoczy, L., Humanized Immunoglobulin Mice: Models for HIV Vaccine Testing and Studying the Broadly Neutralizing Antibody Problem. Adv Immunol, 2017. 134: p. 235-352.
82. Dosenovic, P., et al., Immunization for HIV-1 Broadly Neutralizing Antibodies in Human Ig Knockin Mice. Cell, 2015. 161(7): p. 1505-15.
83. Briney, B., et al., Tailored Immunogens Direct Affinity Maturation toward HIV Neutralizing Antibodies. Cell, 2016. 166(6): p. 1459-1470.e11.
84. Escolano, A., et al., Sequential Immunization Elicits Broadly Neutralizing Anti-HIV-1 Antibodies in Ig Knockin Mice. Cell, 2016. 166(6): p. 1445-1458.e12.
85. Tian, M., et al., Induction of HIV Neutralizing Antibody Lineages in Mice with Diverse Precursor Repertoires. Cell, 2016. 166(6): p. 1471-1484.e18.
86. Yunna, C., et al., Macrophage M1/M2 polarization. European journal of pharmacology, 2020. 877: p. 173090.
87. Connors, J., et al., Using the power of innate immunoprofiling to understand vaccine design, infection, and immunity. Hum Vaccin Immunother, 2023. 19(3): p. 2267295.
88. Connors, J., et al., Aging alters antiviral signaling pathways resulting in functional impairment in innate immunity in response to pattern recognition receptor agonists. Geroscience, 2022: p. 1-18.
89. Alexander, N. O., et al., Monocyte differentiation and macrophage polarization. Vessel Plus, 2019. 3: p. 10.
90. Zhao, Y. L., et al., Comparison of the characteristics of macrophages derived from murine spleen, peritoneal cavity, and bone marrow. J Zhejiang Univ Sci B, 2017. 18(12): p. 1055-1063.
91. Jackaman, C., et al., Targeting macrophages rescues age-related immune deficiencies in C57BL/6J geriatric mice. Aging Cell, 2013. 12(3): p. 345-357.
92. Duong, L., et al., Macrophage function in the elderly and impact on injury repair and cancer. Immun Ageing, 2021. 18(1): p. 4.
93. Soares, H., et al., A subset of dendritic cells induces CD4⁺ T cells to produce IFN-gamma by an IL-12-independent but CD70-dependent mechanism in vivo. J Exp Med, 2007. 204(5): p. 1095-106.
94. Frasca, D., et al., Intrinsic defects in B cell response to seasonal influenza vaccination in elderly humans. Vaccine, 2010. 28(51): p. 8077-8084.
95. Okoli, G. N., et al., Variable seasonal influenza vaccine effectiveness across geographical regions, age groups and levels of vaccine antigenic similarity with circulating virus strains: A systematic review and meta-analysis of the evidence from test-negative design studies after the 2009/10 influenza pandemic. Vaccine, 2021. 39(8): p. 1225-1240.
96. Potluri, T., et al., Age-associated changes in the impact of sex steroids on influenza vaccine responses in males and females. npj Vaccines, 2019. 4(1): p. 29.
97. Fisman, D. N., D. Agrawal, and K. Leder, Effect of Age on Immunologic Response to Recombinant Hepatitis B Vaccine: A Meta-analysis. Clinical Infectious Diseases, 2002. 35(11): p. 1368-1375.
98. Van Der Meeren, O., et al., Characterization of an age-response relationship to GSK's recombinant hepatitis B vaccine in healthy adults: An integrated analysis. Human Vaccines & Immunotherapeutics, 2015. 11(7): p. 1725-1728.
99. Collier, D. A., et al., Age-related immune response heterogeneity to SARS-CoV-2 vaccine BNT162b2. Nature, 2021. 596(7872): p. 417-422.
100. Boehmer, E. D., et al., Age-dependent decrease in Toll-like receptor 4-mediated proinflammatory cytokine production and mitogen-activated protein kinase expression. J Leukoc Biol, 2004. 75(2): p. 342-9.
101. Inamizu, T., M. P. Chang, and T. Makinodan, Influence of age on the production and regulation of interleukin-1 in mice. Immunology, 1985. 55(3): p. 447-55.
102. Wallace, P. K., et al., Decreases in macrophage mediated antitumor activity with aging. Mech Ageing Dev, 1995. 77(3): p. 169-84.
103. Plackett, T. P., et al., Aging and innate immune cells. J Leukoc Biol, 2004. 76(2): p. 291-9.
104. Corsini, E., et al., A defective protein kinase C anchoring system underlying age-associated impairment in TNF-alpha production in rat macrophages. J Immunol, 1999. 163(6): p. 3468-73.
105. Renshaw, M., et al., Cutting edge: impaired Toll-like receptor expression and function in aging. The Journal of Immunology, 2002. 169(9): p. 4697-4701.
106. Ortega, E., J. J. Garcia, and M. De la Fuente, Modulation of adherence and chemotaxis of macrophages by norepinephrine. Influence of ageing. Mol Cell Biochem, 2000. 203(1-2): p. 113-7.
107. Swift, M. E., et al., Age-related alterations in the inflammatory response to dermal injury. J Invest Dermatol, 2001. 117(5): p. 1027-35.
108. Stout-Delgado, H. W., et al., Aging impairs IFN regulatory factor 7 up-regulation in plasmacytoid dendritic cells during TLR9 activation. J Immunol, 2008. 181(10): p. 6747-56.
109. Kissin, E., et al., Age-related decline in murine macrophage production of nitric oxide. J Infect Dis, 1997. 175(4): p. 1004-7.
110. Alvarez, E., et al., Nitric oxide and superoxide anion production decrease with age in resident and activated rat peritoneal macrophages. Cell Immunol, 1996. 169(1): p. 152-5.
111. Antonini, J. M., et al., Effect of age on respiratory defense mechanisms: pulmonary bacterial clearance in Fischer 344 rats after intratracheal instillation of Listeria monocytogenes. Chest, 2001. 120(1): p. 240-9.
112. Albright, J. W. and J. F. Albright, Ageing alters the competence of the immune system to control parasitic infection. Immunol Lett, 1994. 40(3): p. 279-85.
113. Seth, A., et al., Macrophages but not B cells from aged mice are defective in stimulating autoreactive T cells in vitro. Mech Ageing Dev, 1990. 52(2-3): p. 107-24.
114. Garg, M., et al., Cellular basis of decreased immune responses to pneumococcal vaccines in aged mice. Infect Immun, 1996. 64(11): p. 4456-62.
115. Effros, R. B. and R. L. Walford, The effect of age on the antigen-presenting mechanism in limiting dilution precursor cell frequency analysis. Cell Immunol, 1984. 88(2): p. 531-9.
116. Haruna, H., et al., Abnormalities of B cells and dendritic cells in SAMP1 mice. Eur J Immunol, 1995. 25(5): p. 1319-25.
117. Zissel, G., M. Schlaak, and J. Miiller-Quernheim, Age-related decrease in accessory cell function of human alveolar macrophages. J Investig Med, 1999. 47(1): p. 51-6.
118. Theobald, S. J., et al., Spleen tyrosine kinase mediates innate and adaptive immune crosstalk in SARS-CoV-2 mRNA vaccination. EMBO Molecular Medicine, 2022. 14(8): p. e15888.
119. Yang, S.-J., et al., Activation of M1 Macrophages in Response to Recombinant TB Vaccines With Enhanced Antimycobacterial Activity. Frontiers in Immunology, 2020. 11.
120. Quiding-Jarbrink, M., S. Raghavan, and M. Sundquist, Enhanced M1 macrophage polarization in human Helicobacter pylori-associated atrophic gastritis and in vaccinated mice. PLoS One, 2010. 5(11): p. e15018.
121. Zahavi, D. and J. W. Hodge, Targeting Immunosuppressive Adenosine Signaling: A Review of Potential Immunotherapy Combination Strategies. Int J Mol Sci, 2023. 24(10).
122. Vigano, S., et al., Targeting Adenosine in Cancer Immunotherapy to Enhance T-Cell Function. Frontiers in Immunology, 2019. 10(925).
123. Watkins, B. A., et al., Expression of CD26 does not correlate with the replication of macrophage-tropic strains of HIV-1 in T-cell lines. Virology, 1996. 224(1): p. 276-280.
124. Gary, E. N., et al., Adenosine deaminase augments SARS-CoV-2 specific cellular and humoral responses in aged mouse models of immunization and challenge. Front Immunol, 2023. 14: p. 1138609.
125. Blanco, P., et al., Dendritic cells and cytokines in human inflammatory and autoimmune diseases. Cytokine Growth Factor Rev, 2008. 19(1): p. 41-52.
126. Chomarat, P., et al., TNF skews monocyte differentiation from macrophages to dendritic cells. J Immunol, 2003. 171(5): p. 2262-9.
127. Clark, J., et al., What does tumour necrosis factor excess do to the immune system long term? Ann Rheum Dis, 2005. 64 Suppl 4(Suppl 4): p. iv70-6.
128. Dienz, O., et al., The induction of antibody production by IL-6 is indirectly mediated by IL-21 produced by CD4+ T cells. J Exp Med, 2009. 206(1): p. 69-78.
129. Kimura, A. and T. Kishimoto, IL-6: regulator of Treg/Th17 balance. Eur J Immunol, 2010. 40(7): p. 1830-5.
130. Palatella, M., et al., The dark side of Tregs during aging. Frontiers in Immunology, 2022. 13.
131. Teissier, T., E. Boulanger, and L. S. Cox, Interconnections between Inflammageing and Immunosenescence during Ageing. Cells, 2022. 11(3): p. 359.
132. Galanti, M. and J. Shaman, Direct Observation of Repeated Infections With Endemic Coronaviruses. The Journal of Infectious Diseases, 2020. 223(3): p. 409-415.
133. Lam, V. C. and L. L. Lanier, NK cells in host responses to viral infections. Current Opinion in Immunology, 2017. 44: p. 43-51.
134. Klein, F., et al., Somatic Mutations of the Immunoglobulin Framework Are Generally Required for Broad and Potent HIV-1 Neutralization. Cell, 2013. 153(1): p. 126-138.
135. Asbach, B., et al., Priming with a Potent HIV-1 DNA Vaccine Frames the Quality of Immune Responses prior to a Poxvirus and Protein Boost. J Virol, 2019. 93(3).
136. Lu, S., Heterologous prime-boost vaccination. Curr Opin Immunol, 2009. 21(3): p. 346-51.
137. Caniels, T. G., et al., Germline-targeting HIV-1 Env vaccination induces VRC01-class antibodies with rare insertions. Cell Rep Med, 2023. 4(4): p. 101003.
138. Hraber, P., et al., Prevalence of broadly neutralizing antibody responses during chronic HIV-1 infection. Aids, 2014. 28(2): p. 163-9.
139. Ohno, S., N. Mori, and T. Matsunaga, Antigen-binding specificities of antibodies are primarily determined by seven residues of VH. Proc Natl Acad Sci USA, 1985. 82(9): p. 2945-9.
140. Burton, D. R., Advancing an HIV vaccine; advancing vaccinology. Nature Reviews Immunology, 2019. 19(2): p. 77-78.
141. Mandapathil, M., et al., CD26 expression and adenosine deaminase activity in regulatory T cells (Treg) and CD4(+) T effector cells in patients with head and neck squamous cell carcinoma. Oncoimmunology, 2012. 1(5): p. 659-669.
142. Krzyszczyk, P., et al., The Role of Macrophages in Acute and Chronic Wound Healing and Interventions to Promote Pro-wound Healing Phenotypes. Front Physiol, 2018. 9: p. 419.
143. Leone, R. D. and L. A. Emens, Targeting adenosine for cancer immunotherapy. Journal for immunotherapy of cancer, 2018. 6(1): p. 57-57.
144. Franco, R., et al., Adenosine Receptor Antagonists to Combat Cancer and to Boost Anti-Cancer Chemotherapy and Immunotherapy. Cells, 2021. 10(11): p. 2831.
145. Klysz, D. D., et al., Inosine Induces Stemness Features in CAR T cells and Enhances Potency. bioRxiv, 2023: p. 2023.04.21.537859.
146. Cusimano, Gina Marie. Exploring Adenosine Deaminase 1 (ADA-1) as a Novel Molecular Vaccine Adjuvant. Diss. Drexel University, 2024.

### Example IV: ADA-1 Modulates Wound Healing

Given the known roles of pro-inflammatory macrophages in initiating wound healing processes, especially angiogenesis, this defective pro-inflammatory behavior could be a major reason for the impaired wound healing observed in elderly patients. Moreover, to date no therapeutics have been developed that rescue the defective early pro-inflammatory phase in elderly wound healing. Therefore, identified herein is a novel adjuvant-containing biomaterial will rescue the impaired inflammatory response and accelerate wound healing in elderly individuals.

Pro-inflammatory macrophages are critical for wound healing, especially by initiating angiogenesis and recruiting other immune cells to the wound site [5, 20]. In addition, that pro-inflammatory polarization primes macrophages to transition to a reparative phenotype in response to the critical wound healing cytokine, interleukin-4 (IL-4) [21]. Pro-inflammatory macrophages that switched to a reparative phenotype exhibited enhanced regenerative capabilities compared to macrophages that were not first polarized to a pro-inflammatory phenotype. This underscores a second mechanism by which dysfunctional pro-inflammatory activation of macrophages can contribute to improper wound healing.

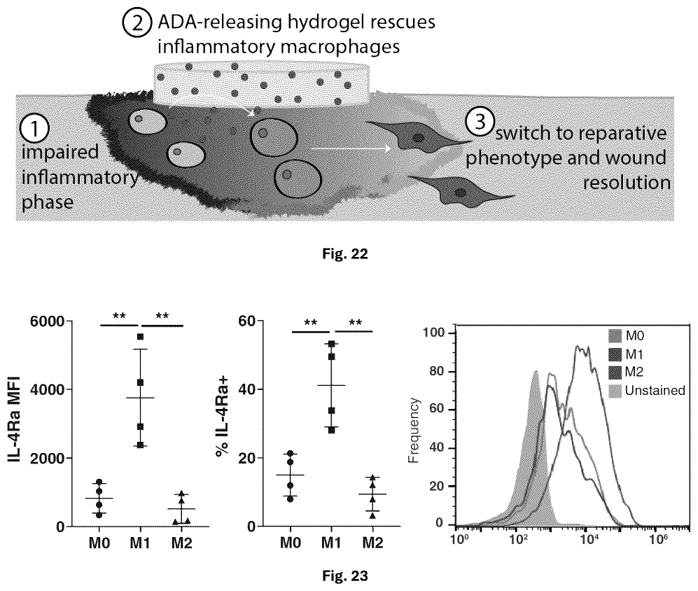

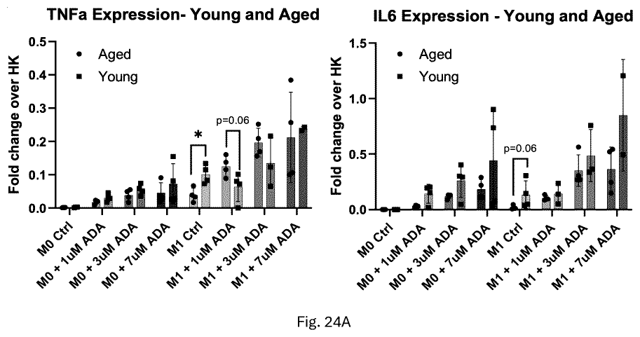

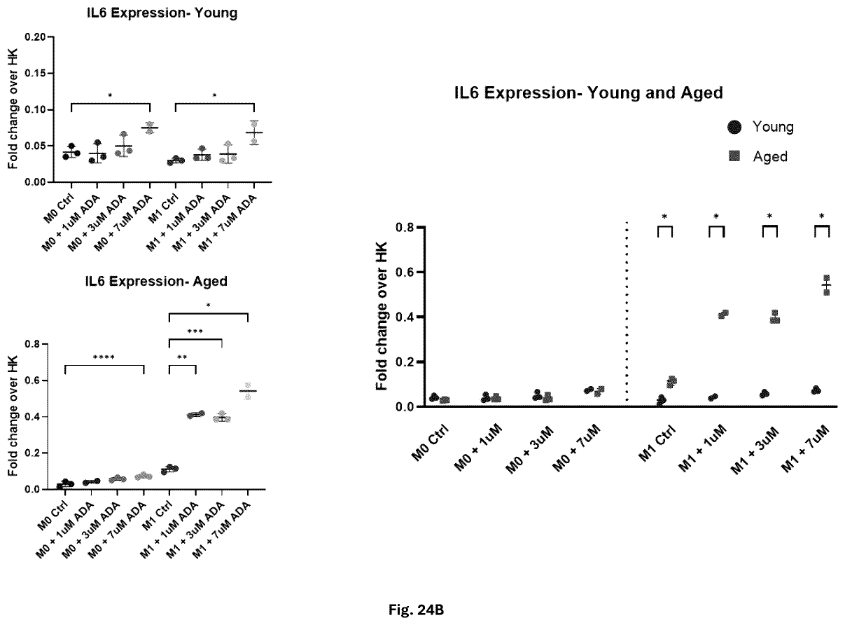

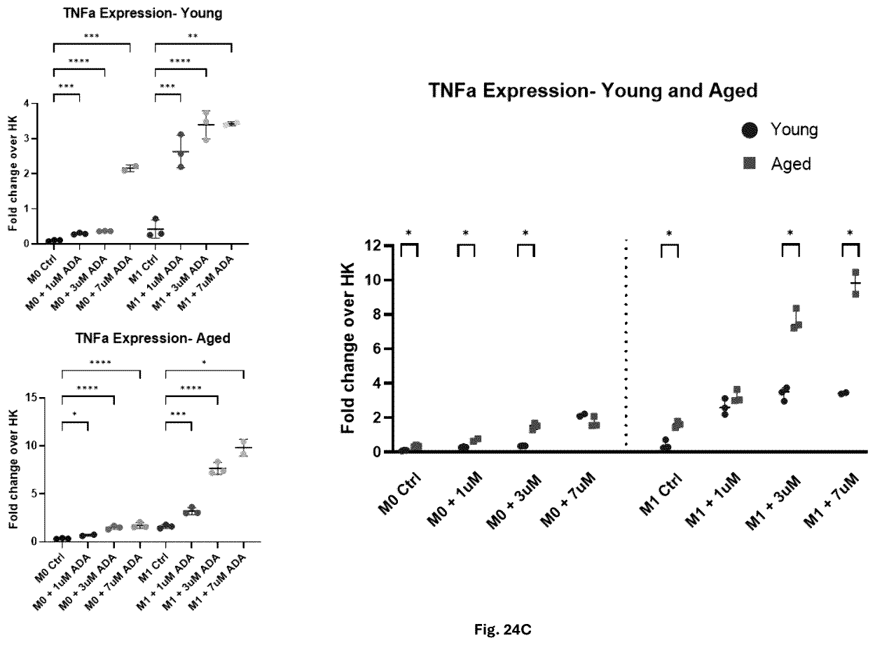

The data presented herein indicates that adenosine deaminase 1 (ADA) can rescue the defective inflammatory response of primary human macrophages from elderly donors to pro-inflammatory stimuli, namely lipopolysaccharide (LPS) and interferon-gamma (IFNγ). Accordingly, a gelatin-based hydrogel was designed that releases ADA gradually over 1-2 days to boost the initial inflammatory response and allow the natural pro-inflammatory to reparative transition of macrophages to proceed (FIG. 22). This biomaterial designed specifically for elderly wound healing is expected to restore the defective immune cell profile characteristic of aging. Here, we investigated the mechanisms behind ADA priming macrophages in vitro, while advancing this novel wound dressing towards clinical translation.

In this investigation, we characterize how ADA-releasing hydrogels restore macrophage phenotype transitions in vitro; hypothesizing that ADA rescues the pro-inflammatory response of aged macrophages to stimuli at early timepoints and boosts the subsequent reparative phenotype in response to IL-4. Additionally, we determine the immunomodulatory effects of ADA-releasing hydrogels to accelerate wound healing in cutaneous wounds of aged mice; hypothesizing that ADA-releasing hydrogels will accelerate wound closure in vivo by rescuing the inflammatory macrophage phenotype to promote angiogenesis and enabling the subsequent transition to reparative phenotype, resulting in proper wound healing.

### Immunomodulatory Biomaterials Hold Promise for Healing Chronic Wounds

Wound healing in young patients progresses through tightly controlled phases, with inflammation and proliferation regulated primarily by macrophages. Dysregulation in the macrophage population has been shown to result in improper wound healing, prompting the development of pro-reparative biomaterials. However, the data above shows that pro-inflammatory macrophages are primed to transition into a pro-reparative phenotype. They not only upregulate the primary receptor for the main pro-reparative phenotype cytokine, IL-4 (FIG. 23) but are more sensitive to IL-4 and exhibit enhanced regenerative functions after exposure to IL-4, including angiogenic processes. These results indicate that not only is pro-inflammatory polarization of macrophages important for initiation of angiogenesis in wound healing, but it is also important for enabling the subsequent reparative phenotype of macrophages. Macrophages in elderly patients are known to be less responsive to pro-inflammatory stimuli compared to macrophages from younger individuals. Therefore, designing an immunomodulatory biomaterial that enhances the initial pro-inflammatory phase may be critical for successful healing of chronic wounds in the elderly.

ADA is a Novel Vaccine Adjuvant that Rescues the Inflammatory Macrophage Phenotype from Elderly Patients.

We explored whether ADA could rescue the impaired pro-inflammatory behavior in macrophages from elderly individuals by stimulating primary human macrophages from young and elderly donors. We found that ADA rescued gene expression of TNFα and IL-6, key pro-inflammatory genes, by aged donor-derived macrophages to levels expressed by human macrophages from young patients (FIG. 23). Additionally, ADA did not decrease expression of key reparative genes like CCL22 (data not shown). These data indicate that ADA could rescue the impaired inflammatory macrophage phenotype in aged patients.

### Research Design and Methods

We analyze the extent to which ADA, delivered from a GelMA hydrogel, rescues the response of aged human macrophages to pro-inflammatory stimuli comparted to young macrophages in vitro. We further analyze whether ADA rescues the subsequent response to the pro-reparative cytokine, IL-4 and characterize how ADA-releasing hydrogel influence wound healing in a murine splinted wound model in aged mice.

### Characterization of ADA-Releasing Hydrogels Restoring Macrophage Phenotype Transitions In Vitro.

We determine how ADA-releasing hydrogels rescue the inflammatory phenotype of aged macrophages in vitro using gene expression and protein secretion analysis. We selected a hydrogel because they are commonly used in wound healing. GelMA was used because its properties can be manipulated to control the diffusivity and subsequent release of proteins from the hydrogel [34, 35]. We further determine the minimum required dose of ADA on the hydrogel to rescue the inflammatory response in aged human macrophages. IL-4 is added to characterize the phenotype transitions of aged macrophages in comparison to young macrophages. The ADA rescues the pro-inflammatory response of aged macrophages to stimuli at early timepoints and boosts the subsequent reparative phenotype in response to IL-4. The outcome of this experiment includes understanding how ADA restores the natural pro-inflammatory-to-pro-reparative phenotype transitions in macrophages.

To obtain this outcome, first, we determine the minimum dose of human ADA releasing from GelMA hydrogels necessary to rescue the inflammatory phenotype in macrophages from elderly donors (aged macrophages) in comparison to macrophages from young donors (young macrophages) when treated with the pro-inflammatory stimuli, LPS and IFNα, for 24 and 48 hours. Then, we change the media to IL-4-containing media for an additional 3 days to promote the reparative phenotype. Macrophage phenotype is characterized over time on both the gene and protein level using quantitative real-time PCR (qRT-PCR) and enzyme-linked immunosorbent assays (ELISAs), respectively, according to our previously described methods [20, 21, 36-39]. Additionally, experiments are conducted using murine bone marrow derived macrophages (BMDMs) to ensure that human derived ADA-releasing hydrogels can be used in vivo in murine models. Importantly, we already confirmed that human ADA effectively promotes pro-inflammatory polarization of young and aged murine macrophages in vitro.

### Materials and Methods

Macrophage culture: BMDMs are harvested from young and aged, male and female, C57BL/6 mice. Human peripheral blood mononuclear cells (PBMCs) from both young (<60 years old) and aged (>70 years old), male and female, human donors are isolated from buffy coats using negative selection beads. To differentiate into macrophages, BMDMs and PBMCs are cultured according to routine methods [5, 20, 21, 40]. ADA-releasing hydrogels with three different doses, plus blank controls, are prepared by encapsulating ADA within hydrogels according to previous methods for manufacturing GelMA hydrogels [34, 35]. ADA release is characterized over time in PBS using ELISAs. Macrophages are cultured on top of the ADA-releasing hydrogels in the presence or absence of the pro-inflammatory stimuli, LPS and IFNγ, for 48 hours. Cell culture supernatants are collected for ELISAs, and macrophages are directly lysed for RNA extraction for qRT-PCR. Some hydrogels are further cultured in the presence of the pro-reparative cytokine, IL-4, and similarly characterized over time at 1 and 3 days later. Protein and gene markers of the pro-inflammatory phenotype include TNFα, interleukin-6 (IL-6), interleukin-1β (IL-1β), and vascular endothelial growth factor-A (VEGFA). Markers of the pro-reparative phenotype include platelet-derived growth-factor-BB (PDGFBB), transforming growth factor beta-1 (TGFB1), CC motif chemokine ligand (CCL) 17 (CCL17), CCL18, and CCL22.

Rigor and reproducibility: We use 2 different and complementary methods to characterize the phenotype of macrophages over time: gene expression and protein secretion. Additionally, after determining the dose of ADA, the study is repeated with a larger sample number at one dose of ADA to confirm reproducibility.

Statistical and power analysis: Prior to analysis, all outcome measures are summarized with descriptive statistics, distributions examined, and variables transformed as appropriate. We use two-way ANOVA with fixed effects (i.e. group, time, and interactions). Assuming similar effect sizes as in our preliminary studies, we have n=8 replicates for gene expression profiling. These replicates provide statistical power (1−β) of 80% at significance level of α=5%. All assays are run in triplicate. Experiments are conducted with equal numbers of male and female sources; if potential sex differences are identified, the experiments are repeated with appropriate numbers of replicates to definitively define these differences.

This experiment determines if controlled release of the novel adjuvant, ADA, rescues the inflammatory phase in aged macrophages and prime the switch from the pro-inflammatory to the reparative phenotype after subsequent exposure to IL-4. The data presented herewith indicates that aged macrophages stimulated with ADA recover an inflammatory response like that of macrophages from young donors using both gene expression and protein secretion as measures of success. Furthermore, we have also previously demonstrated that inflammatory macrophages are primed towards the reparative phenotype stimulated by IL-4 [21]. A more robust reparative macrophage phenotype is present from macrophages treated with ADA, then stimulated with IL-4, compared to macrophages that do not receive ADA before the IL-4 stimulus, as measured by higher expression of reparative markers. In summary, by rescuing the inflammatory phenotype, aged macrophages will behave as young, healthy macrophages and switch to the reparative phenotype after being stimulated with ADA.

IFNγ, IL-3, and IL-4 can be added to the hydrogel to further promote the inflammatory macrophage phenotype. In certain embodiments, IL-13 will be added with IL-4 at day 3 to further stimulate macrophages towards a reparative phenotype, recapitulating the presence of multiple reparative cytokines in the wound bed as healing progresses.

### Example V: Immunomodulatory Effects of ADA-1

The timing of the stimulus with an ADA-releasing hydrogel that promotes wound closure in vivo is analyzed in a splinted wound model in aged mice. Our data shows that aged macrophages are stimulated by ADA, effectively rescuing the inflammatory phenotype. Therefore, analyzed herein is the hypothesis that ADA-releasing hydrogels accelerate wound closure in vivo by rescuing the inflammatory macrophage phenotype to promote angiogenesis and enabling the subsequent transition to reparative phenotype, resulting in proper wound healing. This experiment allows for characterizing wound healing over time in aged mice by measuring angiogenesis and new tissue formation using immunohistochemistry and by identifying macrophage phenotype over time using flow cytometry.

Experimental design: Murine wounds heal differently from human wounds in that superficial wound closure in mice occurs primarily by contraction. Therefore, a splinted wound model is used to prevent contraction, such that wound healing occurs primarily by re-epithelialization, as in humans [43-45]. On the day of injury, aged mice receive either a GelMA hydrogel or a GelMA hydrogel loaded with ADA, with 8 mice per treatment group. Wound closure is monitored over time by photographing the wound daily and evaluating changes in wound size. Angiogenesis is evaluated by analyzing cluster of differentiation 31 positive (CD31+) structures using immunohistochemistry and re-epithelialization is measured from hematoxylin and eosin (H&E) staining. Macrophage phenotype is tracked by excising the entire wound and evaluating intracellular and extracellular markers using flow cytometry.

### Methods

Splinted skin wound model. All animal experiments are conducted in accordance with protocols approved by Drexel University's Institutional Animal Care and Use Committee. The splinted skin wound model in aged C57BL/6 mice (18 months old) is conducted according to standard methods [46-48]. Two wounds, one on each side of the midline, are made on the shaved dorsum of the mice using a sterile single-use disposable 6 mm punch biopsy tool pushed through the skin folded at the midline. Full-thickness wounds extending through the panniculus carnosus is excised and silicone rings are sutured to the skin to splint the wounds and minimize contraction-mediated healing. One wound on each mouse is treated with an ADA-releasing hydrogel while the other is treated with the vehicle control (i.e. hydrogel without ADA) and covered with fresh Tegaderm. Daily photographs and area measurements are collected over a 28-day period. On days 1, 3, 7, 14, and 28 post-surgery, an 8 mm punch biopsy tool is used to excise the entire wound area and surrounding tissue, which is then processed for either flow cytometry to evaluate host immune cell phenotype (days 1, 3, 7) or histology/immunohistochemistry to quantify reepithelization and angiogenesis (days 14, 28).

Immune cell phenotyping. Macrophage phenotype is tracked by excising the entire wound on days 1, 3, and 7, post-surgery and evaluating intracellular and extracellular markers using multidimensional analysis of a 14-color phenotype panel that we have previously described [40]. This flow cytometry panel includes a pan-macrophage marker (F4/80), pro-inflammatory markers (CD38, CD86, PD-L1, CXCR4), IL4-driven pro-reparative markers (CD206, CD301b, RELMa, Arg1), and other phenotype markers (CD163, Tie2, MerTK, etc.).

Wound healing. Wound closure is monitored daily by photographing the wound and evaluating the size of the wound is compared to the standard plastic ring size using ImageJ. Tissue structure is evaluated on days 14 and 28 post-surgery using H&E staining. Wound repair scores is then assigned based on the degree of re-epithelialization, granulation tissue formation, and collagen organization. Angiogenesis is measured by quantifying the number and diameter of CD31+ and alpha smooth muscle actin (aSMA) positive structures (blood vessels).

Rigor and reproducibility. Quality control of ADA-releasing hydrogels is assessed by release and degradation studies using a subset of hydrogels created for the in vivo study. Additionally, this is repeated to confirm reproducibility.

Statistical andpower analysis: Prior to analysis, all outcome measures are summarized with descriptive statistics, distributions examined, and variables transformed as appropriate. We use two-way ANOVA with fixed effects (i.e. group, time, and interactions). Assuming similar effect sizes as in our previous studies, we use at least n=8 replicates for phenotyping by flow cytometry and n=8 for effects on angiogenesis and wound healing as measured by gross wound imaging, histology, and immunohistochemistry for blood vessels. These replicates provide statistical power (1−β) of 80% at significance level of α=5%. All assays are run in triplicate. Experiments are conducted with equal numbers of male and female mice; if potential sex differences are identified, we repeat the experiments with appropriate numbers of replicates to definitively define these differences.

The results demonstrate the use of a novel adjuvant early in wound healing to rescue the inflammatory phase and accelerate wound healing in aged mice. ADA-releasing hydrogels cause an early (days 1 and 3) increase in inflammatory activity in macrophages followed by a later (day 7) shift to reparative phenotype, compared to wounds treated with blank hydrogels. This shift in immune profile is associated with increased angiogenesis and faster wound closure in aged mice. If wound healing is not accelerated as expected in vivo or the effects on macrophage phenotype are not seen in vivo, the dose of ADA loaded into the hydrogel is increased.

### REFERENCES

1. Franceschi, C. and J. Campisi, Chronic Inflammation (Inflammaging) and Its Potential Contribution to Age-Associated Diseases. The Journals of Gerontology: Series A, 2014. 69(Suppl_1): p. S4-S9.
2. Guimarães, G. R., et al., Hallmarks of Aging in Macrophages: Consequences to Skin Inflammaging. Cells, 2021. 10(6): p. 1323.
3. Agrawal, R., A. Hu, and W. B. Bollag, The Skin and Inflamm-Aging. Biology, 2023. 12(11): p. 1396.
4. Khalid, K. A., et al., Aging and Wound Healing of the Skin: A Review of Clinical and Pathophysiological Hallmarks. Life, 2022. 12(12): p. 2142.
5. Spiller, K. L., et al., The role of macrophage phenotype in vascularization of tissue engineering scaffolds. Biomaterials, 2014. 35(15): p. 4477-4488.
6. Mosser, D. M. and J. P. Edwards, Exploring the full spectrum of macrophage activation. Nature Reviews Immunology, 2008. 8(12): p. 958-969.
7. Arnold, L., et al., Inflammatory monocytes recruited after skeletal muscle injury switch into antiinflammatory macrophages to support myogenesis. Journal of Experimental Medicine, 2007. 204(5): p. 1057-1069.
8. Liu, G., et al., Phenotypic and functional switch of macrophages induced by regulatory CD4+ CD25+ T cells in mice. Immunology and cell biology, 2011. 89(1): p. 130-142.
9. Dube, C. T., et al., Age-Related Alterations in Macrophage Distribution and Function Are Associated With Delayed Cutaneous Wound Healing. Frontiers in Immunology, 2022. 13.
10. Aprahamian, T., et al., Ageing is associated with diminished apoptotic cell clearance in vivo. Clinical and Experimental Immunology, 2008. 152(3): p. 448-455.
11. Arnardottir, H. H., et al., Aging Delays Resolution of Acute Inflammation in Mice: Reprogramming the Host Response with Novel Nano-Proresolving Medicines. The Journal of Immunology, 2014. 193(8): p. 4235-4244.
12. Deng, Y., et al., Inflammation-Instructed Hierarchical Delivery of IL-4/miR-21 Orchestrates Osteoimmune Microenvironment toward the Treatment of Rheumatoid Arthritis. Advanced Functional Materials, 2021. 31(33): p. 2101033.
13. Cottler, P. S., et al., Immunomodulation of Acellular Dermal Matrix Through Interleukin 4 Enhances Vascular Infiltration. Annals of Plastic Surgery, 2022. 88(5): p. S466-S472.
14. Hachim, D., et al., Shifts in macrophage phenotype at the biomaterial interface via IL-4 eluting coatings are associated with improved implant integration. Biomaterials, 2017. 112: p. 95-107.
15. Tian, G., et al., Cell-free decellularized cartilage extracellular matrix scaffolds combined with interleukin 4 promote osteochondral repair through immunomodulatory macrophages: In vitro and in vivo preclinical study. Acta Biomaterialia, 2021. 127: p. 131-145.
16. Gomez, C. R., et al., Interleukin-6 Contributes to Age-Related Alteration of Cytokine Production by Macrophages. Mediators of Inflammation, 2010. 2010: p. 1-7.
17. Mahbub, S., C. R. Deburghgraeve, and E. J. Kovacs, Advanced Age Impairs Macrophage Polarization. Journal of Interferon &amp; Cytokine Research, 2012. 32(1): p. 18-26.
18. Chelvarajan, R. L., et al., Molecular basis of age-associated cytokine dysregulation in LPS-stimulated macrophages. Journal of Leukocyte Biology, 2006. 79(6): p. 1314-1327.
19. Boehmer, E. D., et al., Aging negatively skews macrophage TLR2- and TLR4-mediated pro-inflammatory responses without affecting the IL-2-stimulated pathway. Mechanisms of Ageing and Development, 2005. 126(12): p. 1305-1313.
20. Graney, P. L., et al., Macrophages of diverse phenotypes drive vascularization of engineered tissues. Science Advances, 2020. 6(18): p. eaay6391.
21. O'Brien, E. M. and K. L. Spiller, Pro-inflammatory polarization primes Macrophages to transition into a distinct M2-like phenotype in response to IL-4. Journal of Leukocyte Biology, 2022. 111(5): p. 989-1000.
22. Gary, E., et al., Adenosine deaminase-1 enhances germinal center formation and functional antibody responses to HIV-1 Envelope DNA and protein vaccines. Vaccine, 2020. 38(22): p. 3821-3831.
23. Cusimano, G. M., et al., Improved Durability to SARS-CoV-2 Vaccine Immunity following Coimmunization with Molecular Adjuvant Adenosine Deaminase-1. The Journal of Immunology, 2022: p. ji2200056.
24. Gary, E. N., et al., Adenosine deaminase augments SARS-CoV-2 specific cellular and humoral responses in aged mouse models of immunization and challenge. Frontiers in Immunology, 2023. 14.
25. Nussbaum, S. R., et al., An Economic Evaluation of the Impact, Cost, and Medicare Policy Implications of Chronic Nonhealing Wounds. Value in Health, 2018. 21(1): p. 27-32.
26. Verma, K. D., et al., Food and Drug Administration perspective: Advancing product development for non-healing chronic wounds. Wound Repair and Regeneration, 2022. 30(3): p. 299-302.
27. DiPietro, L. A., Wound healing: the role of the macrophage and other immune cells. Shock (Augusta, Ga.), 1995. 4(4): p. 233-240.
28. Hachim, D., et al., Distinct macrophage populations and phenotypes associated with IL-4 mediated immunomodulation at the host implant interface. Biomaterials Science, 2020. 8(20): p. 5751-5762.
29. Buckley, R. H., Molecular Defects in Human Severe Combined Immunodeficiency and Approaches to Immune Reconstitution. Annual Review of Immunology, 2004. 22(1): p. 625-655.
30. Qu, Y., et al., Adenosine Deaminase 1 Overexpression Enhances the Antitumor Efficacy of Chimeric Antigen Receptor-Engineered T Cells. Human Gene Therapy, 2021. 33(5-6): p. 223-236.
31. Flinn, A. M. and A. R. Gennery, Adenosine deaminase deficiency: a review. Orphanet Journal of Rare Diseases, 2018. 13(1): p. 65.
32. Tardif, V., et al., Adenosine deaminase-1 delineates human follicular helper T cell function and is altered with HIV. Nature Communications, 2019. 10(1): p. 823.
33. Pacheco, R., et al., CD26, adenosine deaminase, and adenosine receptors mediate costimulatory signals in the immunological synapse. Proceedings of the National Academy of Sciences of the United States of America, 2005. 102(27): p. 9583-9588.
34. Ersumo, N., C. E. Witherel, and K. L. Spiller, Differences in time-dependent mechanical properties between extruded and molded hydrogels. Biofabrication, 2016. 8(3): p. 035012.
35. Witherel, C. E., et al., Regulation of extracellular matrix assembly and structure by hybrid M1/M2 macrophages. Biomaterials, 2021. 269: p. 120667.
36. Wofford, K. L., et al., Biomaterial-mediated reprogramming of monocytes via microparticle phagocytosis for sustained modulation of macrophage phenotype. Acta Biomaterialia, 2020. 101: p. 237-248.
37. Lurier, E. B., et al., Imparting Immunomodulatory Activity to Scaffolds via Biotin-Avidin Interactions. ACS Biomaterials Science & Engineering, 2021.
38. Tylek, T., et al., Biomaterial-mediated intracellular control of macrophages for cell therapy in pro-inflammatory and pro-fibrotic conditions. Biomaterials, 2024. 308: p. 122545.
39. Steele, L. A., B. Hernaez Estrada, and K. L. Spiller, Effects of a Bioengineered Allogeneic Cellularized Construct (BACC) on Primary Human Macrophage Phenotype. Advanced Healthcare Materials, 2024.
40. Risser, G. E., et al., Effects of Interleukin-4 (IL-4)-releasing microparticles and adoptive transfer of macrophages on immunomodulation and angiogenesis. Biomaterials, 2023. 296: p. 122095.
41. Gurtner, G. C., et al., Wound repair and regeneration. Nature, 2008. 453(7193): p. 314-321.
42. Gurevich, D. B., et al., Live imaging of wound angiogenesis reveals macrophage orchestrated vessel sprouting and regression. The EMBO Journal, 2018. 37(13): p. e97786.
43. Hori, K., et al., Impaired cutaneous wound healing in transforming growth factor-β inducible early gene1 knockout mice. Wound Repair and Regeneration, 2012. 20(2): p. 166-177.
44. Jagetia, G. C. and G. K. Rajanikant, Acceleration of wound repair by curcumin in the excision wound of mice exposed to different doses of fractionated <i>γ</i> radiation. International Wound Journal, 2012. 9(1): p. 76-92.
45. Naldaiz-Gastesi, N., et al., Identification and Characterization of the Dermal Panniculus Carnosus Muscle Stem Cells. Stem Cell Reports, 2016. 7(3): p. 411-424.
46. Dunn, L., et al., Murine Model of Wound Healing. Journal of Visualized Experiments, 2013(75).
47. Martin, K. E., et al., Hydrolytic hydrogels tune mesenchymal stem cell persistence and immunomodulation for enhanced diabetic cutaneous wound healing. Biomaterials, 2023. 301: p. 122256.
48. Jimi, S., et al., A Novel Skin Splint for Accurately Mapping Dermal Remodeling and Epithelialization During Wound Healing. Journal of Cellular Physiology, 2017. 232(6): p. 1225-1232.

### Embodiments

1. A vaccine composition comprising
a) a DNA vaccine, and
b) a polynucleotide encoding Adenosine deaminase-1 (ADA-1) comprising the sequence of SEQ ID NO: 1 (human), a sequence sharing at least 90% identity therewith, SEQ ID NO: 3 (mouse), or a sequence sharing at least 90% identity therewith.

2. The vaccine composition of embodiment 1, wherein the DNA vaccine is generated using a triple tandem trimer platform.

3. The composition of embodiment 1 or 2, wherein the DNA vaccine is pBG505-TTT.

4. The composition of any one of embodiments 1-3, wherein the polynucleotide encoding ADA-1 is present in a plasmid, which is optionally a plasmid or viral vector, or wherein the polynucleotide is comprised in a lipid nanoparticle.

5. The composition of any one of embodiments 1-6, wherein the polynucleotide encodes an ADA-1 fusion protein comprising an IgE signal peptide and an ADA-1.

6. The composition of any one of embodiments 1-5, further comprising a pharmaceutically acceptable carrier.

7. A method of increasing vaccine response in a subject, comprising administering an ADA-1 polypeptide in combination with a vaccine composition to the subject, wherein administration enhances macrophage cytokine and chemokine profile.

8. The method of embodiment 7, wherein administration increases secretion of anti-inflammatory cytokines and/or chemokines in macrophages.

9. The method of embodiment 7 or 8, wherein administration enhances TNFα, IL-6, IFNγ, and/or IL2 expression in the subject when compared to a subject treated with just the vaccine composition.

10. The method of any one of embodiments 7-9, wherein administration modulates CCL17, CCL22, CCL24 and/or CXCL13 expression.

11. The method of any one of embodiments 7-10, wherein administration increases the magnitude, durability, isotype switching and/or functionality of antigen-specific antibodies.

12. The method of any one of embodiments 7-11, wherein administration enhances antigen-specific T cell polyfunctionality, activation, and degranulation when compared to a subject treated without the ADA-1.

13. The method of any one of embodiments 7-12, wherein treatment increases antibody dependent complement deposition (ADCD).

14. The method of any one of embodiments 7-13, wherein treatment drives IgG2a or IgG2b expression.

15. The method of any one of embodiments 7-14, wherein the subject is an adult over 60 years of age.

16. The method of any one of embodiments 7-15, wherein the vaccine composition is a COVID-19 vaccine or an HIV-1 vaccine.

17. The method of any one of embodiments 16, wherein the vaccine composition is an HIV-1 vaccine and administration induces bNAbs against HIV.

18. The method of any one of embodiments 7-17, wherein the vaccine composition is selected from the any one of embodiments 1-6.

19. A method of inducing bNabs against a disease in a subject in need thereof, the method comprising administering a vaccine composition and an adjuvant comprising a polynucleotide encoding Adenosine deaminase-1 (ADA-1) comprising the sequence of SEQ ID NO: 1 (human), a sequence sharing at least 90% identity therewith, SEQ ID NO: 3 (mouse), or a sequence sharing at least 90% identity therewith.

20. The method of embodiment 19, further comprising detecting an increased level bNabs in the subject when compared to a control treated without the adjuvant.

21. A method of increasing antibody dependent complement deposition (ADCD) a subject in need thereof, the method comprising administering a vaccine composition and an adjuvant comprising a polynucleotide encoding Adenosine deaminase-1 (ADA-1) comprising the sequence of SEQ ID NO: 1 (human), a sequence sharing at least 90% identity therewith, SEQ ID NO: 3 (mouse), or a sequence sharing at least 90% identity therewith.

22. The method of embodiment 21, further comprising detecting an increase in ADCD in the subject when compared to a control treated without the adjuvant.

23. A method of enhancing antigen-specific T cell polyfunctionality, activation, or degranulation a subject in need thereof, the method comprising administering a vaccine composition and an adjuvant comprising a polynucleotide encoding Adenosine deaminase-1 (ADA-1) comprising the sequence of SEQ ID NO: 1 (human), a sequence sharing at least 90% identity therewith, SEQ ID NO: 3 (mouse), or a sequence sharing at least 90% identity therewith.

24. The method of embodiment 23, further comprising detecting an increase in antigen-specific T cell polyfunctionality, activation or degranulation in the subject when compared to a control treated without the adjuvant.

25. The method of any one of embodiments 19-24, wherein the disease is HIV-1.

26. A hydrogel composition comprising a polynucleotide encoding Adenosine deaminase-1 (ADA-1) or an ADA-1 peptide.

27. The hydrogel composition of embodiment 26, wherein the polynucleotide encoding ADA-1 comprises the sequence of SEQ ID NO: 1 (human), a sequence sharing at least 90% identity therewith, SEQ ID NO: 3 (mouse), or a sequence sharing at least 90% identity therewith, and the ADA-1 peptide comprises the sequence of SEQ ID NO: 2, a sequence sharing at least 90% identity therewith, SEQ ID NO: 4, or a sequence sharing at least 90% identity therewith.

28. The hydrogel composition of embodiment 26, wherein the hydrogel is a gelatin-based hydrogel.

29. The hydrogel composition of embodiment 26, wherein the hydrogel is a GelMA hydrogel.

30. The hydrogel composition of embodiment 26, wherein the ADA-1 is released over one or more days.

31. The hydrogel composition of embodiment 26, wherein the ADA-1 is released over 1-2 days.

32. The hydrogel composition of embodiment 26, wherein the hydrogel restores macrophage phenotype transitions.

33. The hydrogel composition of embodiment 26, further comprising at least one additional therapeutic.

34. The hydrogel composition of embodiment 32, wherein the at least one additional therapeutic is selected from an antibiotic, pain reliever, anti-inflammatory agent, or a steroid.

35. A method of treating a wound comprising administering a hydrogel composition comprising a polynucleotide encoding Adenosine deaminase-1 (ADA-1) or an ADA-1 peptide.

36. The method of embodiment 35, wherein the polynucleotide encoding ADA-1 comprises the sequence of SEQ ID NO: 1 (human), a sequence sharing at least 90% identity therewith, SEQ ID NO: 3 (mouse), or a sequence sharing at least 90% identity therewith, and the ADA-1 peptide comprises the sequence of SEQ ID NO: 2, a sequence sharing at least 90% identity therewith, SEQ ID NO: 4, or a sequence sharing at least 90% identity therewith.

37. The method of embodiment 35, wherein the wound is a chronic wound, an acute wound, an open wound, a closed wound, a clean wound, a contaminated wound, an infected wound, a diabetic wound, an ulcer, a diabetic ulcer, a foot sore, or a skin sore.

38. The method of embodiment 35, wherein the hydrogel is a gelatin-based hydrogel.

39. The method of embodiment 35, wherein the hydrogel is a GelMA hydrogel.

40. The method of embodiment 35, wherein the ADA-1 is released over one or more days.

41. The method of embodiment 35, wherein the ADA-1 is released over 1-2 days.

42. The method of embodiment 35, wherein the hydrogel restores macrophage phenotype transitions.

43. The method of embodiment 35, further comprising at least one additional therapeutic.

44. The method of embodiment 43, wherein the at least one additional therapeutic is selected from an antibiotic, pain reliever, anti-inflammatory agent, or a steroid.

1. A hydrogel composition comprising an Adenosine deaminase-1 (ADA-1) peptide.

2. The hydrogel composition of claim 1, wherein the ADA-1 peptide is SEQ ID NO: 2, a sequence sharing at least 90% identity therewith, SEQ ID NO: 4, or a sequence sharing at least 90% identity therewith.

3. The hydrogel composition of claim 1, wherein the hydrogel is a gelatin-based hydrogel.

4. The hydrogel composition of claim 1, wherein the hydrogel is a GelMA hydrogel.

5. The hydrogel composition of claim 1, which is formulated such that the ADA-1 protein is released over one or more days.

6. The hydrogel composition of claim 1, which is formulated such that the ADA-1 protein is released over 1-2 days.

7. The hydrogel composition of claim 1, further comprising at least one additional therapeutic.

8. The hydrogel composition of claim 7, wherein the at least one additional therapeutic is selected from an antibiotic, pain reliever, anti-inflammatory agent, or a steroid.

9. A method of treating a wound comprising administering a hydrogel composition comprising an Adenosine deaminase-1 (ADA-1) peptide.

10. The method of claim 9, wherein the ADA-1 peptide is SEQ ID NO: 2, a sequence sharing at least 90% identity therewith, SEQ ID NO: 4, or a sequence sharing at least 90% identity therewith.

11. The method of claim 9, wherein administering said hydrogel composition accelerates wound healing when compared to an untreated wound.

12. The method of claim 9, wherein the wound is a chronic wound, an acute wound, an open wound, a closed wound, a clean wound, a contaminated wound, an infected wound, a diabetic wound, an ulcer, a diabetic ulcer, a foot sore, or a skin sore.

13. The method of claim 9, wherein the hydrogel is a gelatin-based hydrogel.

14. The method of claim 9, wherein the hydrogel is a GelMA hydrogel.

15. The method of claim 9, wherein the ADA-1 peptide is released over one or more days.

16. The method of claim 9, wherein the ADA-1 peptide is released over 1-2 days.

17. The method of claim 9, wherein the hydrogel restores macrophage phenotype transitions.

18. The method of claim 9, further comprising at least one additional therapeutic.

19. The method of claim 18, wherein the at least one additional therapeutic is selected from an antibiotic, pain reliever, anti-inflammatory agent, or a steroid.

20. A method of inducing bNabs against a disease in a subject in need thereof, the method comprising administering a vaccine composition and an adjuvant comprising a polynucleotide encoding Adenosine deaminase-1 (ADA-1) comprising the sequence of SEQ ID NO: 1, a sequence sharing at least 90% identity therewith, SEQ ID NO: 3, or a sequence sharing at least 90% identity therewith.
